SEQUENCE LISTING (24 sequences)

SEQ ID NO: 1 - protein, 1946 aa, synthetic construct
  REGION 1..1946: note = Synthesized
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAPASSEITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLYDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - protein, 883 aa, synthetic construct
  REGION 1..883: note = Synthesized
SLSCRPPMVKLVCPADNLRAEGLECTKTCQNYDLECMSMGCVSGCLCPPGMVRHENRCVALERCPCFHQGKEYAPGETVKIGCNTCVCRDRKWNCTDHVCDATCSTIGMAHYLTFDGLKYLFPGECQYVLVQDYCGSNPGTFRILVGNKGCSHPSVKCKKRVTILVEGGEIELFDGEVNVKRPMKDETHFEVVESGRYIILLLGKALSVVWDRHLSISVVLKQTYQEKVCGLCGNFDGIQNNDLTSSNLQVEEDPVDFGNSWKVSSQCADTRKVPLDSSPATCHNNIMKQTMVDSSCRILTSDVFQDCNKLVDPEPYLDVCIYDTCSCESIGDCAAFCDTIAAYAHVCAQHGKVVTWRTATLCPQSCEERNLRENGYEAEWRYNSCAPACQVTCQHPEPLACPVQCVEGCHAHCPPGKILDELLQTCVDPEDCPVCEVAGRRFASGKKVTLNPSDPEHCQICHCDVVNLTCEACQEPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGASSDKNTGDYYEDSYEDISAYLLSKNNAIEPRSFSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 3 - protein, 1965 aa, synthetic construct
  REGION 1..1965: note = Synthesized
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAPASSEITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLYDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 4 - DNA, 5898 bp, synthetic construct
  misc_feature 1..5898: note = Synthesized
  source 1..5898: mol_type = other DNA
atgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctctcaaaacggtacctcagagtctgctacccccgagtcagggccaggatcagagccagccacctccgggtctgagacacccgggacttccgagagtgccacccctgagtccggacccgggtccgagcccgccacttccggctccgaaactcccggcacaagcgagagcgctaccccagagtcaggaccaggaacatctacagagccctctgaaggctccgctccagggtccccagccggcagtcccactagcaccgaggagggaacctctgaaagcgccacacccgaatcagggccagggtctgagcctgctaccagcggcagcgagacaccaggcacctctgagtccgccacaccagagtccggacccggatctcccgctgggagccccacctccactgaggagggatctcctgctggctctccaacatctactgaggaaggaacctcaaccgagccatccgagggatcagctcccggcacctcagagtcggcaaccccggagtctggacccggaacttccgaaagtgccacaccagagtccggtcccgggacttcagaatcagcaacacccgagtccggccctgggtctgaacccgccacaagtggtagtgagacaccaggatcagaacctgctacctcagggtcagagacacccggatctccggcaggctcaccaacctccactgaggagggcaccagcacagaaccaagcgagggctccgcacccggaacaagcactgaacccagtgagggttcagcacccggctctgagccggccacaagtggcagtgagacacccggcacttcagagagtgccacccccgagagtggcccaggcactagtaccgagccctctgaaggcagtgcgccagcctcgagcgaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctacgacaaaactcacacatgcccaccgtgcccagctccagaactcctgggcggaccgtcagtcttcctcttccccccaaaacccaaggacaccctcatgatctcccggacccctgaggtcacatgcgtggtggtggacgtgagccacgaagaccctgaggtcaagttcaactggtatgtggacggcgtggaagtgcataatgccaagacaaagccgcgggaggagcagtacaacagcacgtaccgtgtggtcagcgtcctcaccgtcctgcaccaagactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaagttagcctgacctgcctggtcaaaggcttctatcccagcgacatcgccgtggagtgggagagcaatgggcagccggagaacaactacaagaccacgcctcccgtgttggactccgacggctccttcttcctctactccaagctcaccgtggacaagagcaggtggcagcaggggaacgtcttctcatgctccgtgatgcatgaggctctgcacaaccactacacgcagaagagcctctccctgtctccgggttga

SEQ ID NO: 5 - protein, 1646 aa, synthetic construct
  REGION 1..1646: note = Synthesized
MIPARFAGVLLALALILPGTLCAEGTRGRSSTARCSLFGSDFVNTFDGSMYSFAGYCSYLLAGGCQKRSFSIIGDFQNGKRVSLSVYLGEFFDIHLFVNGTVTQGDQRVSMPYASKGLYLETEAGYYKLSGEAYGFVARIDGSGNFQVLLSDRYFNKTCGLCGNFNIFAEDDFMTQEGTLTSDPYDFANSWALSSGEQWCERASPPSSSCNISSGEMQKGLWEQCQLLKSTSVFARCHPLVDPEPFVALCEKTLCECAGGLECACPALLEYARTCAQEGMVLYGWTDHSACSPVCPAGMEYRQCVSPCARTCQSLHINEMCQERCVDGCSCPEGQLLDEGLCVESTECPCVHSGKRYPPGTSLSRDCNTCICRNSQWICSNEECPGECLVTGQSHFKSFDNRYFTFSGICQYLLARDCQDHSFSIVIETVQCADDRDAVCTRSVTVRLPGLHNSLVKLKHGAGVAMDGQDIQLPLLKGDLRIQHTVTASVRLSYGEDLQMDWDGRGRLLVKLSPVYAGKTCGLCGNYNGNQGDDFLTPSGLAEPRVEDFGNAWKLHGDCQDLQKQHSDPCALNPRMTRFSEEACAVLTSPTFEACHRAVSPLPYLRNCRYDVCSCSDGRECLCGALASYAAACAGRGVRVAWREPGRCELNCPKGQVYLQCGTPCNLTCRSLSYPDEECNEACLEGCFCPPGLYMDERGDCVPKAQCPCYYDGEIFQPEDIFSDHHTMCYCEDGFMHCTMSGVPGSLLPDAVLSSPLSHRSKRSLSCRPPMVKLVCPADNLRAEGLECTKTCQNYDLECMSMGCVSGCLCPPGMVRHENRCVALERCPCFHQGKEYAPGETVKIGCNTCVCRDRKWNCTDHVCDATCSTIGMAHYLTFDGLKYLFPGECQYVLVQDYCGSNPGTFRILVGNKGCSHPSVKCKKRVTILVEGGEIELFDGEVNVKRPMKDETHFEVVESGRYIILLLGKALSVVWDRHLSISVVLKQTYQEKVCGLCGNFDGIQNNDLTSSNLQVEEDPVDFGNSWKVSSQCADTRKVPLDSSPATCHNNIMKQTMVDSSCRILTSDVFQDCNKLVDPEPYLDVCIYDTCSCESIGDCAAFCDTIAAYAHVCAQHGKVVTWRTATLCPQSCEERNLRENGYEAEWRYNSCAPACQVTCQHPEPLACPVQCVEGCHAHCPPGKILDELLQTCVDPEDCPVCEVAGRRFASGKKVTLNPSDPEHCQICHCDVVNLTCEACQEPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGASSDKNTGDYYEDSYEDISAYLLSKNNAIEPRSFSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 6 - DNA, 4941 bp, synthetic construct
  misc_feature 1..4941: note = Synthesized
  source 1..4941: mol_type = other DNA
atgattcctgccagatttgccggggtgctgcttgctctggccctcattttgccagggaccctttgtgcagaaggaactcgcggcaggtcatccacggcccgatgcagccttttcggaagtgacttcgtcaacacctttgatgggagcatgtacagctttgcgggatactgcagttacctcctggcagggggctgccagaaacgctccttctcgattattggggacttccagaatggcaagagagtgagcctctccgtgtatcttggggaattttttgacatccatttgtttgtcaatggtaccgtgacacagggggaccaaagagtctccatgccctatgcctccaaagggctgtatctagaaactgaggctgggtactacaagctgtccggtgaggcctatggctttgtggccaggatcgatggcagcggcaactttcaagtcctgctgtcagacagatacttcaacaagacctgcgggctgtgtggcaactttaacatctttgctgaagatgactttatgacccaagaagggaccttgacctcggacccttatgactttgccaactcatgggctctgagcagtggagaacagtggtgtgaacgggcatctcctcccagcagctcatgcaacatctcctctggggaaatgcagaagggcctgtgggagcagtgccagcttctgaagagcacctcggtgtttgcccgctgccaccctctggtggaccccgagccttttgtggccctgtgtgagaagactttgtgtgagtgtgctggggggctggagtgcgcctgccctgccctcctggagtacgcccggacctgtgcccaggagggaatggtgctgtacggctggaccgaccacagcgcgtgcagcccagtgtgccctgctggtatggagtataggcagtgtgtgtccccttgcgccaggacctgccagagcctgcacatcaatgaaatgtgtcaggagcgatgcgtggatggctgcagctgccctgagggacagctcctggatgaaggcctctgcgtggagagcaccgagtgtccctgcgtgcattccggaaagcgctaccctcccggcacctccctctctcgagactgcaacacctgcatttgccgaaacagccagtggatctgcagcaatgaagaatgtccaggggagtgccttgtcactggtcaatcccacttcaagagctttgacaacagatacttcaccttcagtgggatctgccagtacctgctggcccgggattgccaggaccactccttctccattgtcattgagactgtccagtgtgctgatgaccgcgacgctgtgtgcacccgctccgtcaccgtccggctgcctggcctgcacaacagccttgtgaaactgaagcatggggcaggagttgccatggatggccaggacatccagctccccctcctgaaaggtgacctccgcatccagcatacagtgacggcctccgtgcgcctcagctacggggaggacctgcagatggactgggatggccgcgggaggctgctggtgaagctgtcccccgtctatgccgggaagacctgcggcctgtgtgggaattacaatggcaaccagggcgacgacttccttaccccctctgggctggcggagccccgggtggaggacttcgggaacgcctggaagctgcacggggactgccaggacctgcagaagcagcacagcgatccctgcgccctcaacccgcgcatgaccaggttctccgaggaggcgtgcgcggtcctgacgtcccccacattcgaggcctgccatcgtgccgtcagcccgctgccctacctgcggaactgccgctacgacgtgtgctcctgctcggacggccgcgagtgcctgtgcggcgccctggccagctatgccgcggcctgcgcggggagaggcgtgcgcgtcgcgtggcgcgagccaggccgctgtgagctgaactgcccgaaaggccaggtgtacctgcagtgcgggaccccctgcaacctgacctgccgctctctctcttacccggatgaggaatgcaatgaggcctgcctggagggctgcttctgccccccagggctctacatggatgagaggggggactgcgtgcccaaggcccagtgcccctgttactatgacggtgagatcttccagccagaagacatcttctcagaccatcacaccatgtgctactgtgaggatggcttcatgcactgtaccatgagtggagtccccggaagcttgctgcctgacgctgtcctcagcagtcccctgtctcatcgcagcaaaaggagcctatcctgtcggccccccatggtcaagctggtgtgtcccgctgacaacctgcgggctgaagggctcgagtgtaccaaaacgtgccagaactatgacctggagtgcatgagcatgggctgtgtctctggctgcctctgccccccgggcatggtccggcatgagaacagatgtgtggccctggaaaggtgtccctgcttccatcagggcaaggagtatgcccctggagaaacagtgaagattggctgcaacacttgtgtctgtcgggaccggaagtggaactgcacagaccatgtgtgtgatgccacgtgctccacgatcggcatggcccactacctcaccttcgacgggctcaaatacctgttccccggggagtgccagtacgttctggtgcaggattactgcggcagtaaccctgggacctttcggatcctagtggggaataagggatgcagccacccctcagtgaaatgcaagaaacgggtcaccatcctggtggagggaggagagattgagctgtttgacggggaggtgaatgtgaagaggcccatgaaggatgagactcactttgaggtggtggagtctggccggtacatcattctgctgctgggcaaagccctctccgtggtctgggaccgccacctgagcatctccgtggtcctgaagcagacataccaggagaaagtgtgtggcctgtgtgggaattttgatggcatccagaacaatgacctcaccagcagcaacctccaagtggaggaagaccctgtggactttgggaactcctggaaagtgagctcgcagtgtgctgacaccagaaaagtgcctctggactcatcccctgccacctgccataacaacatcatgaagcagacgatggtggattcctcctgtagaatccttaccagtgacgtcttccaggactgcaacaagctggtggaccccgagccatatctggatgtctgcatttacgacacctgctcctgtgagtccattggggactgcgccgcattctgcgacaccattgctgcctatgcccacgtgtgtgcccagcatggcaaggtggtgacctggaggacggccacattgtgcccccagagctgcgaggagaggaatctccgggagaacgggtatgaggctgagtggcgctataacagctgtgcacctgcctgtcaagtcacgtgtcagcaccctgagccactggcctgccctgtgcagtgtgtggagggctgccatgcccactgccctccagggaaaatcctggatgagcttttgcagacctgcgttgaccctgaagactgtccagtgtgtgaggtggctggccggcgttttgcctcaggaaagaaagtcaccttgaatcccagtgaccctgagcactgccagatttgccactgtgatgttgtcaacctcacctgtgaagcctgccaggagccgggtacatcagagagcgccacccctgaaagtggtcccgggagcgagccagccacatctgggtcggaaacgccaggcacatccgagtctgcaactcccgagtccggacctggctccgagcctgccactagcggctccgagactccgggaacttccgagagcgctacaccagaaagcggacccggaaccagtaccgaacctagcgagggctctgctccgggcagcccagccggctctcctacatccacggaggagggcacttccgaatccgccaccccggagtcagggccaggatctgaacccgctacctcaggcagtgagacgccaggaacgagcgagtccgctacaccggagagtgggccagggagccctgctggatctcctacgtccactgaggaagggtcaccagcgggctcgcccaccagcactgaagaaggtgcctcgtctgacaagaacactggtgattattacgaggacagttatgaagatatttcagcatacttgctgagtaaaaacaatgccattgaaccaagaagcttctctgacaaaactcacacatgcccaccgtgcccagctccagaactcctgggcggaccgtcagtcttcctcttccccccaaaacccaaggacaccctcatgatctcccggacccctgaggtcacatgcgtggtggtggacgtgagccacgaagaccctgaggtcaagttcaactggtatgtggacggcgtggaagtgcataatgccaagacaaagccgcgggaggagcagtacaacagcacgtaccgtgtggtcagcgtcctcaccgtcctgcaccaagactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaagttagcctgacctgcctggtcaaaggcttctatcccagcgacatcgccgtggagtgggagagcaatgggcagccggagaacaactacaagaccacgcctcccgtgttggactccgacggctccttcttcctctactccaagctcaccgtggacaagagcaggtggcagcaggggaacgtcttctcatgctccgtgatgcatgaggctctgcacaaccactacacgcagaagagcctctccctgtctccgggttga

SEQ ID NO: 7 - protein, 1945 aa, synthetic construct
  REGION 1..1945: note = Synthesized
TRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAPASSEITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLYDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 8 - protein, 2332 aa, Homo sapiens
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSTESGRLFKGKRAHGPALLTKDNALFKVSISLLKTNKTSNNSATNRKTHIDGPSLLIENSPSVWQNILESDTEFKKVTPLIHDRMLMDKNATALRLNHMSNKTTSSKNMEMVQQKKEGPIPPDAQNPDMSFFKMLFLPESARWIQRTHGKNSLNSGQGPSPKQLVSLGPEKSVEGQNFLSEKNKVVVGKGEFTKDVGLKEMVFPSSRNLFLTNLDNLHENNTHNQEKKIQEEIEKKETLIQENVVLPQIHTVTGTKNFMKNLFLLSTRQNVEGSYDGAYAPVLQDFRSLNDSTNRTKKHTAHFSKKGEEENLEGLGNQTKQIVEKYACTTRISPNTSQQNFVTQRSKRALKQFRLPLEETELEKRIIVDDTSTQWSKNMKHLTPSTLTQIDYNEKEKGAITQSPLSDCLTRSHSIPQANRSPLPIAKVSSFPSIRPIYLTRVLFQDNSSHLPAASYRKKDSGVQESSHFLQGAKKNNLSLAILTLEMTGDQREVGSLGTSATNSVTYKKVENTVLPKPDLPKTSGKVELLPKVHIYQKDLFPTETSNGSPGHLDLVEGSLLQGTEGAIKWNEANRPGKVPFLRVATESSAKTPSKLLDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 9 - protein, 288 aa, synthetic construct
  REGION 1..288: note = Synthesized
GTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAP

SEQ ID NO: 10 - protein, 46 aa, synthetic construct
  REGION 1..46: note = Synthesized
TSTEEGASISDKNTGDYYEDSYEDISAYLLSKNNAIEPRSFSDKTH

SEQ ID NO: 11 - protein, 45 aa, synthetic construct
  REGION 1..45: note = Synthesized
TSTEEGASSDKNTGDYYEDSYEDISAYLLSKNNAIEPRSFSDKTH

SEQ ID NO: 12 - protein, 1947 aa, synthetic construct
  REGION 1..1947: note = Synthesized
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAPASSEITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLYDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 288 aa, synthetic construct
  REGION 1..288: note = Synthesized
GSPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAP

SEQ ID NO: 14 - protein, 144 aa, synthetic construct
  REGION 1..144: note = Synthesized
TSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEG

SEQ ID NO: 15 - protein, 32 aa, synthetic construct
  REGION 1..32: note = Synthesized
DKNTGDYYEDSYEDISAYLLSKNNAIEPRSFS

SEQ ID NO: 16 - protein, 19 aa, Homo sapiens
MQIELSTCFFLCLLRFCFS

SEQ ID NO: 17 - protein, 745 aa, Homo sapiens
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQN

SEQ ID NO: 18 - protein, 684 aa, Homo sapiens
EITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 19 - protein, 22 aa, Homo sapiens
MIPARFAGVLLALALILPGTLC

SEQ ID NO: 20 - protein, 741 aa, Homo sapiens
AEGTRGRSSTARCSLFGSDFVNTFDGSMYSFAGYCSYLLAGGCQKRSFSIIGDFQNGKRVSLSVYLGEFFDIHLFVNGTVTQGDQRVSMPYASKGLYLETEAGYYKLSGEAYGFVARIDGSGNFQVLLSDRYFNKTCGLCGNFNIFAEDDFMTQEGTLTSDPYDFANSWALSSGEQWCERASPPSSSCNISSGEMQKGLWEQCQLLKSTSVFARCHPLVDPEPFVALCEKTLCECAGGLECACPALLEYARTCAQEGMVLYGWTDHSACSPVCPAGMEYRQCVSPCARTCQSLHINEMCQERCVDGCSCPEGQLLDEGLCVESTECPCVHSGKRYPPGTSLSRDCNTCICRNSQWICSNEECPGECLVTGQSHFKSFDNRYFTFSGICQYLLARDCQDHSFSIVIETVQCADDRDAVCTRSVTVRLPGLHNSLVKLKHGAGVAMDGQDIQLPLLKGDLRIQHTVTASVRLSYGEDLQMDWDGRGRLLVKLSPVYAGKTCGLCGNYNGNQGDDFLTPSGLAEPRVEDFGNAWKLHGDCQDLQKQHSDPCALNPRMTRFSEEACAVLTSPTFEACHRAVSPLPYLRNCRYDVCSCSDGRECLCGALASYAAACAGRGVRVAWREPGRCELNCPKGQVYLQCGTPCNLTCRSLSYPDEECNEACLEGCFCPPGLYMDERGDCVPKAQCPCYYDGEIFQPEDIFSDHHTMCYCEDGFMHCTMSGVPGSLLPDAVLSSPLSHRSKR

SEQ ID NO: 21 - protein, 103 aa, synthetic construct
  REGION 1..103: note = Synthesized
SLSCRPPMVKLVCPADNLRAEGLECTKTCQNYDLECMSMGCVSGCLCPPGMVRHENRCVALERCPCFHQGKEYAPGETVKIGCNTCVCRDRKWNCTDHVCDAT

SEQ ID NO: 22 - protein, 374 aa, synthetic construct
  REGION 1..374: note = Synthesized
CSTIGMAHYLTFDGLKYLFPGECQYVLVQDYCGSNPGTFRILVGNKGCSHPSVKCKKRVTILVEGGEIELFDGEVNVKRPMKDETHFEVVESGRYIILLLGKALSVVWDRHLSISVVLKQTYQEKVCGLCGNFDGIQNNDLTSSNLQVEEDPVDFGNSWKVSSQCADTRKVPLDSSPATCHNNIMKQTMVDSSCRILTSDVFQDCNKLVDPEPYLDVCIYDTCSCESIGDCAAFCDTIAAYAHVCAQHGKVVTWRTATLCPQSCEERNLRENGYEAEWRYNSCAPACQVTCQHPEPLACPVQCVEGCHAHCPPGKILDELLQTCVDPEDCPVCEVAGRRFASGKKVTLNPSDPEHCQICHCDVVNLTCEACQEP

SEQ ID NO: 23 - protein, 226 aa, synthetic construct
  REGION 1..226: note = Synthesized
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 24 - protein, 291 aa, synthetic construct
  REGION 1..291: note = Synthesized
GTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAPASS

(19) United States
(12) Patent Application Publication
(10) Pub. No.: US 2024/0358800 A1
(43) Pub. Date: Oct. 31, 2024

(54) FORMULATIONS OF FACTOR VIII CHIMERIC PROTEINS AND USES THEREOF

(71) Applicant: BIOVERATIV THERAPEUTICS INC., Waltham, MA (US)

(72) Inventors: Tyler CARLAGE, Waltham, MA (US); Randall MAULDIN, Waltham, MA (US); Timothy MCCOY, Waltham, MA (US); Loubna Mzaalak TAZI, Waltham, MA (US)

(21) Appl. No.: 18/632,038

(22) Filed: Apr. 10, 2024

Related U.S. Application Data
(63) Continuation of application No. PCT/US2022/078097, filed on Oct. 14, 2022.
(60) Provisional application No. 63/256,432, filed on Oct. 15, 2021.

(51) Int. Cl. A61K 38/37; A61K 9/00; A61K 9/19; A61K 47/02; A61K 47/10; A61K 47/18; A61K 47/22; A61K 47/26

(52) U.S. Cl. CPC A61K 38/37; A61K 9/0019; A61K 9/19; A61K 47/02; A61K 47/10; A61K 47/183; A61K 47/22; A61K 47/26

(57) ABSTRACT

The present disclosure provides pharmaceutical compositions of a chimeric protein comprising a factor VIII (FVIII) polypeptide and a von Wilobrand factor (VWF) polypeptide. Also disclosed are pharmaceutical kits and methods of using the disclosed pharmaceutical compositions to treat hemophilia A.

### RELATED APPLICATIONS

This application is a continuation of International Patent Application No. PCT/US2022/078097, filed Oct. 14, 2022, which claims priority to U.S. Provisional Patent Application Ser. No. 63/256,432, filed Oct. 15, 2021, the contents of which are incorporated herein in their entirety.

### REFERENCE TO SEQUENCE LISTING SUBMITTED ELECTRONICALLY

The content of the electronically submitted sequence listing in XML file format (Name: 749710_SA9-483PCCON_ST26.xml; Size: 55,885 bytes; Created: Apr. 10, 2024) is incorporated herein by reference in its entirety.

### BACKGROUND OF THE DISCLOSURE

Hemophilia A is a bleeding disorder caused by defects in the gene encoding coagulation factor VIII (FVIII) and affects 1-2 in 10,000 male births. Graw et al., Nat. Rev. Genet. 6 (6): 488-501 (2005). Patients affected with hemophilia A can be treated with infusions of purified plasma FVIII or recombinantly produced FVIII. Many commercially available FVIII products are known to have a half-life of about 8-12 hours, requiring frequent intravenous administration to the patients. See Weiner M. A. and Cairo, M. S., Pediatric Hematology Secrets, Lee, M. T., 12. Disorders of Coagulation, Elsevier Health Sciences, 2001; Lillicrap, D. Thromb. Res. 122 Suppl 4: S2-8 (2008). In addition, a number of approaches have been tried in order to extend the FVIII half-life. For example, the approaches in development to extend the half-life of clotting factors include pegylation, glycopegylation, and conjugation with albumin. See Dumont et al., Blood. 119 (13): 3024-3030 (2012). Consistent results have been demonstrated in humans, for example, rFVIIIFc was reported to improve half-life up to ˜1.7-fold compared with ADVATER in hemophilia A patients. See Powell et al., Blood. 119 (13): 3031-3037 (2012). Therefore, the half-life increases, despite minor improvements, indicate the presence of other half-life limiting factors, such as clearance by VWF. Pipe et al., Blood. 128 (16): 2007-2016 (2016)).

Efanesoctocog alfa (also known as Efa and BIVV001) is a fusion protein that is designed to uncouple recombinant clotting factor VIII from VWF in circulation. The chimeric protein comprises a single recombinant factor VIII protein fused to dimeric Fc, a D′D3 domain of VWF, and two ELNN Polypeptides. Chhabra et al. Blood 135 (17): 1484-1496 (2020). In one early study involving patients with severe hemophilia A, a single intravenous injection of efanesoctocog alfa resulted in high sustained factor VIII activity levels, with a half-life that was up to four times the half-life associated with recombinant factor VIII. See Konkle et al., NEJM. 383:1018-27 (2020).

However, there remains a need for improved pharmaceutical compositions.

### SUMMARY OF THE DISCLOSURE

The present disclosure is directed to, inter alia, pharmaceutical compositions comprising a Factor VIII (“FVIII”) protein, kits comprising such pharmaceutical compositions, and therapeutic methods and uses of the pharmaceutical compositions.

In some embodiments, the pharmaceutical composition comprises a chimeric protein or protein which comprises a first polypeptide chain which comprises a FVIII protein or a portion thereof and a first immunoglobulin (“Ig”) constant region or a portion thereof, and a second polypeptide chain which comprises a von Willebrand Factor (“VWF”) protein and a second Ig constant region or a portion thereof. In some embodiments, the chimeric protein comprises (i) a FVIII protein comprising a FVIII polypeptide, an ELNN Polypeptide inserted within the B domain (e.g., replacing at least a portion of the B domain) of the FVIII polypeptide, and a first Fc region; and (ii) a VWF protein comprising a VWF fragment (e.g., a fragment comprising the D′D3 domains of VWF, which fragment may comprise mutations), a second ELNN Polypeptide, a thrombin-cleavable linker (such as an a2 linker), and a second Fc region. In some embodiments, the chimeric protein disclosed herein is a FVIII-ELNN-Fc/D′D3-ELNN-Fc heterodimer.

In some embodiments, the pharmaceutical composition comprises: (a) a FVIII protein; (b) about 1% (w/v) to about 7.5% (w/V) sucrose; (c) about 5 mM to about 15 mM histidine; (d) about 200 mM to about 300 mM arginine; (e) about 2.5 mM to about 10 mM calcium chloride; and (f) about 0.01% (w/v) to about 1.0% (w/v) of a poloxamer. In some embodiments, the pharmaceutical composition comprises: (a) a FVIII protein; (b) about 1% (w/v) to about 7.5% (w/v) sucrose; (c) about 5 mM to about 15 mM L-histidine; (d) about 200 mM to about 300 mM L-arginine; (e) about 2.5 mM to about 10 mM calcium chloride; and (f) about 0.01% (w/v) to about 1.0% (w/v) of a poloxamer. In some embodiments, the pharmaceutical composition comprising: (a) a Factor VIII (“FVIII”) protein; (b) about 1% (w/v) to about 7.5% (w/V) sucrose; (c) about 5 mM to about 15 mM L-histidine; (d) about 200 mM to about 300 mM L-arginine-HCl; (e) about 2.5 mM to about 10 mM calcium chloride dihydrate; and (f) about 0.008% (w/v) to about 0.1% (w/v) of a poloxamer.

In some embodiments, the composition comprises about 250 mM L-arginine. In some embodiments, the composition comprises about 250 mM L-arginine-HCl. In some embodiments, the poloxamer is poloxamer 188 (poloxamer 188, which is also known as P188). Poloxamer 188 is well known in the art.

In some embodiments, disclosed herein is a pharmaceutical composition comprising:
(a) a chimeric protein comprising a first polypeptide chain which comprises a Factor VIII (“FVIII”) protein and a first immunoglobulin (“Ig”) constant region or a portion thereof, and a second polypeptide chain which comprises a von Willebrand Factor (“VWF”) protein and a second Ig constant region or a portion thereof;
(b) about 1% (w/v) to about 7.5% (w/v) sucrose;
(c) about 5 mM to about 15 mM histidine;
(d) about 200 mM to about 300 mM arginine;
(e) about 2.5 mM to about 10 mM calcium chloride; and
(f) about 0.01% (w/v) to about 1.0% (w/v) of poloxamer 188.

In some embodiments, the pharmaceutical composition comprises about 250 IU, 500 IU, 1000 IU, 2000 IU, 3000 IU, or 4,000 IU of the chimeric protein. In some embodiments, the pharmaceutical composition comprises more than about 250 IU of the chimeric protein. In some embodiments, the pharmaceutical composition comprises more than about 300 IU of the chimeric protein. In some embodiments, the pharmaceutical composition comprises more than about 500 IU of the chimeric protein. In some embodiments, the pharmaceutical composition comprises at least about 500 IU of the chimeric protein. In some embodiments, the pharmaceutical composition comprises more than about 85 IU/ml of the chimeric protein. In some embodiments, the pharmaceutical composition comprises more than about 100 IU/ml of the chimeric protein. In some embodiments, the pharmaceutical composition comprises more than about 200 IU/ml of the chimeric protein. In some embodiments, the pharmaceutical composition comprises at least about 200 IU/ml of the chimeric protein.

In some embodiments, the first polypeptide chain comprises the amino acid sequence set forth as SEQ ID NO: 1 and the second polypeptide chain comprises the amino acid sequence set forth as SEQ ID NO: 2, wherein the first polypeptide chain and the second polypeptide chain are covalently linked by two disulfide bonds between Fc domains in the first and second polypeptide chains. In some embodiments, the chimeric protein is efanesoctocog alfa.

In some embodiments, the pharmaceutical composition comprises about 1% (w/v) to about 5% (w/v) sucrose. In some embodiments, the pharmaceutical composition comprises about 5 mM to about 15 mM histidine. In some embodiments, the pharmaceutical composition comprises about 200 mM to about 300 mM arginine. In some embodiments, the pharmaceutical composition comprises about 2.5 mM to about 10 mM calcium chloride. In some embodiments, the pharmaceutical composition comprises poloxamer 188 (P188). In some embodiments, the pharmaceutical composition comprises about 0.01% (w/v) to about 1.0% (w/v) poloxamer 188.

In some embodiments, disclosed herein is a pharmaceutical composition comprising:
(a) about 1% (w/v) to about 5% (w/v) sucrose;
(b) about 5 mM to about 15 mM histidine;
(c) about 200 mM to about 300 mM arginine;
(d) about 2.5 mM to about 10 mM calcium chloride; and
(e) about 0.01% (w/V) to about 1.0% (w/v) poloxamer 188.

In some embodiments, disclosed herein is a pharmaceutical composition comprising:
(a) about 1%, 2%, or 5% (w/v) sucrose;
(b) about 10 mM histidine;
(c) about 250 mM arginine;
(d) about 5 mM calcium chloride; and
(e) about 0.1% (w/v) poloxamer 188.

In some embodiments, disclosed herein is a pharmaceutical composition comprising:
(a) about 5% (w/v) sucrose;
(b) about 10 mM L-histidine;
(c) about 250 mM L-arginine-HCl;
(d) about 5 mM calcium chloride; and
(e) about 0.1% (w/v) poloxamer 188.

In some embodiments, disclosed herein is a pharmaceutical composition comprising:
(a) about 2% (w/v) sucrose;
(b) about 10 mM L-histidine;
(c) about 250 mM L-arginine-HCl;
(d) about 5 mM calcium chloride; and
(e) about 0.1% (w/v) poloxamer 188.

In some embodiments, disclosed herein is a pharmaceutical composition comprising:
(a) about 1% (w/v) sucrose;
(b) about 10 mM L-histidine;
(c) about 250 mM L-arginine-HCl;
(d) about 5 mM calcium chloride; and
(e) about 0.1% (w/v) poloxamer 188.

In some embodiments, disclosed herein is a pharmaceutical composition comprising:
(a) 10 mg/mL to 60 mg/mL sucrose;
(b) 1.5 mg/mL to 2.0 mg/mL L-histidine;
(c) 40 mg/mL to 70 mg/mL L-arginine-HCl;
(d) 0.4 mg/mL to 0.9 mg/mL calcium chloride; and
(e) 0.6 mg/mL to 1.6 mg/mL poloxamer 188.

In some embodiments, disclosed herein is a pharmaceutical composition comprising:
(a) 56.12 mg/ml sucrose;
(b) 1.74 mg/ml L-histidine;
(c) 59.11 mg/ml L-arginine-HCl;
(d) 0.82 mg/ml calcium chloride dihydrate; and
(e) 1.12 mg/ml poloxamer 188.

In some embodiments, disclosed herein is a pharmaceutical composition comprising:
(a) 22.45 mg/ml sucrose;
(b) 1.74 mg/ml L-histidine;
(c) 59.11 mg/ml L-arginine-HCl;
(d) 0.82 mg/ml calcium chloride dihydrate; and
(e) 1.12 mg/ml poloxamer 188.

In some embodiments, disclosed herein is a pharmaceutical composition comprising:
(a) 22.45 mg/ml sucrose;
(b) 1.74 mg/ml L-histidine;
(c) 59.11 mg/ml L-arginine-HCl;
(d) 0.62 mg/ml calcium chloride; and
(e) 1.12 mg/ml poloxamer 188.

In some embodiments, disclosed herein is a pharmaceutical composition comprising:
(a) 11.23 mg/ml sucrose;
(b) 1.74 mg/ml L-histidine;
(c) 59.11 mg/ml L-arginine-HCl;
(d) 0.82 mg/ml calcium chloride dihydrate; and
(e) 1.12 mg/ml poloxamer 188.

In some embodiments, disclosed herein is a pharmaceutical composition comprising:
(a) 11.23 mg/ml sucrose;
(b) 1.74 mg/ml L-histidine;
(c) 59.11 mg/ml L-arginine-HCl;
(d) 0.62 mg/ml calcium chloride; and
(e) 1.12 mg/ml poloxamer 188.

In some embodiments, disclosed herein is a pharmaceutical composition comprising:
(a) 10 mg/mL to 60 mg/mL sucrose;
(b) 1.5 mg/mL to 2.0 mg/mL L-histidine;
(c) 40 mg/mL to 70 mg/mL L-arginine-HCl;
(d) 0.5 mg/mL to 0.9 mg/mL calcium chloride dihydrate; and
(e) 0.6 mg/mL to 1.6 mg/mL poloxamer 188.

In some embodiments, disclosed herein is a pharmaceutical composition comprising:
(a) about 50 mg/ml sucrose;
(b) about 1.6 mg/mL L-histidine;
(c) about 52.7 mg/mL L-arginine-HCl;
(d) about 0.7 mg/mL calcium chloride dihydrate; and
(e) about 1 mg/mL poloxamer 188.

In some embodiments, disclosed herein is a pharmaceutical composition comprising:
(a) about 20 mg/ml sucrose;
(b) about 1.6 mg/mL L-histidine;
(c) about 52.7 mg/mL L-arginine-HCl;
(d) about 0.7 mg/mL calcium chloride dihydrate; and
(e) about 1 mg/mL poloxamer 188.

In some embodiments, disclosed herein is a pharmaceutical composition comprising:
(a) about 20 mg/mL sucrose;
(b) about 1.6 mg/mL L-histidine;
(c) about 52.7 mg/mL L-arginine-HCl;
(d) about 0.6 mg/mL calcium chloride; and
(e) about 1 mg/mL poloxamer 188.

In some embodiments, disclosed herein is a pharmaceutical composition comprising:
(a) about 20 mg/mL sucrose;
(b) about 1.6 mg/mL L-histidine;
(c) about 43.6 mg/mL L-arginine;
(d) about 0.7 mg/mL calcium chloride dihydrate; and
(e) about 1 mg/mL poloxamer 188.

In some embodiments, disclosed herein is a pharmaceutical composition comprising:
(a) about 10 mg/mL sucrose;
(b) about 1.6 mg/mL L-histidine;
(c) about 43.6 mg/mL L-arginine;
(d) about 0.6 mg/mL calcium chloride; and
(e) about 1 mg/mL poloxamer 188.

In some embodiments, disclosed herein is a pharmaceutical composition comprising:
(a) about 10 mg/mL sucrose;
(b) about 1.6 mg/mL L-histidine;
(c) about 52.7 mg/mL L-arginine-HCl;
(d) about 0.7 mg/mL calcium chloride dihydrate; and
(e) about 1 mg/mL poloxamer 188.

In some embodiments, disclosed herein is a pharmaceutical composition comprising:
(a) about 10 mg/ml sucrose;
(b) about 1.6 mg/mL L-histidine;
(c) about 52.7 mg/mL L-arginine-HCl;
(d) about 0.6 mg/mL calcium chloride; and
(e) about 1 mg/ml poloxamer 188.

In some embodiments, disclosed herein is a pharmaceutical composition comprising:
(a) about 10 mg/mL sucrose;
(b) about 1.6 mg/mL L-histidine;
(c) about 43.6 mg/mL L-arginine;
(d) about 0.7 mg/mL calcium chloride dihydrate; and
(e) about 1 mg/mL poloxamer 188.

In some embodiments, disclosed herein is a pharmaceutical composition comprising:
(a) about 10 mg/ml sucrose;
(b) about 1.6 mg/mL L-histidine;
(c) about 43.6 mg/mL L-arginine;
(d) about 0.6 mg/mL calcium chloride; and
(e) about 1 mg/mL poloxamer 188.

In some embodiments, disclosed herein is a pharmaceutical composition comprising:
(a) 10 mg/mL to 60 mg/mL sucrose;
(b) 1.5 mg/mL to 2.0 mg/mL L-histidine;
(c) 50 mg/mL to 70 mg/mL L-arginine-HCl;
(d) 0.7 mg/mL to 0.9 mg/mL calcium chloride dihydrate; and
(e) 0.6 mg/mL to 1.6 mg/mL poloxamer 188.

In some embodiments, disclosed herein is a pharmaceutical composition comprising:
(a) 10 mg/mL to 60 mg/mL sucrose;
(b) 1.5 mg/mL to 2.0 mg/mL L-histidine;
(c) 50 mg/mL to 70 mg/mL L-arginine-HCl;
(d) 0.4 mg/mL to 0.8 mg/mL calcium chloride; and
(e) 0.6 mg/mL to 1.6 mg/mL poloxamer 188.

In some embodiments, disclosed herein is a pharmaceutical composition comprising:
(a) 10 mg/mL to 60 mg/mL sucrose;
(b) 1.5 mg/mL to 2.0 mg/mL L-histidine;
(c) 40 mg/mL to 60 mg/mL L-arginine;
(d) 0.7 mg/mL to 0.9 mg/mL calcium chloride; and
(e) 0.6 mg/mL to 1.6 mg/mL poloxamer 188.

In some embodiments, disclosed herein is a pharmaceutical composition comprising:
(a) 10 mg/mL to 60 mg/mL sucrose;
(b) 1.5 mg/mL to 2.0 mg/mL L-histidine;
(c) 40 mg/mL to 60 mg/mL L-arginine;
(d) 0.4 mg/mL to 0.7 mg/mL calcium chloride dihydrate; and
(e) 0.6 mg/mL to 1.6 mg/mL poloxamer 188.

In some embodiments, disclosed herein is a pharmaceutical composition comprising:
(a) 56.12 mg/ml sucrose;
(b) 1.74 mg/ml L-histidine;
(c) 59.11 mg/ml L-arginine-HCl;
(d) 0.82 mg/ml calcium chloride dihydrate; and
(e) 1.12 mg/ml poloxamer 188.

In some embodiments, disclosed herein is a pharmaceutical composition comprising:
(a) 22.45 mg/ml sucrose;
(b) 1.74 mg/ml L-histidine;
(c) 59.11 mg/ml L-arginine-HCl;
(d) 0.82 mg/ml calcium chloride dihydrate; and
(e) 1.12 mg/ml poloxamer 188.

In some embodiments, disclosed herein is a pharmaceutical composition comprising:
(a) 22.45 mg/ml sucrose;
(b) 1.74 mg/ml L-histidine;
(c) 48.88 mg/ml L-arginine;
(d) 0.82 mg/ml calcium chloride dihydrate; and
(e) 1.12 mg/ml poloxamer 188.

In some embodiments, disclosed herein is a pharmaceutical composition comprising:
(a) 22.45 mg/ml sucrose;
(b) 1.74 mg/ml L-histidine;
(c) 59.11 mg/ml L-arginine-HCl;
(d) 0.62 mg/ml calcium chloride; and
(e) 1.12 mg/ml poloxamer 188.

In some embodiments, disclosed herein is a pharmaceutical composition comprising:
(a) 22.45 mg/ml sucrose;
(b) 1.74 mg/ml L-histidine;
(c) 48.88 mg/ml L-arginine;
(d) 0.62 mg/ml calcium chloride; and
(e) 1.12 mg/ml poloxamer 188.

In some embodiments, disclosed herein is a pharmaceutical composition comprising:
(a) 11.23 mg/ml sucrose;
(b) 1.74 mg/ml L-histidine;
(c) 59.11 mg/ml L-arginine-HCl;
(d) 0.82 mg/ml calcium chloride dihydrate; and
(e) 1.12 mg/ml poloxamer 188.

In some embodiments, disclosed herein is a pharmaceutical composition comprising:
(a) 11.23 mg/ml sucrose;
(b) 1.74 mg/ml L-histidine;
(c) 48.88 mg/ml L-arginine;
(d) 0.82 mg/ml calcium chloride dihydrate; and
(e) 1.12 mg/ml poloxamer 188.

In some embodiments, disclosed herein is a pharmaceutical composition comprising:
(a) 11.23 mg/ml sucrose;
(b) 1.74 mg/ml L-histidine;
(c) 59.11 mg/ml L-arginine-HCl;
(d) 0.62 mg/ml calcium chloride; and
(e) 1.12 mg/ml poloxamer 188.

In some embodiments, disclosed herein is a pharmaceutical composition comprising:
(a) 11.23 mg/ml sucrose;
(b) 1.74 mg/ml L-histidine;
(c) 48.88 mg/ml L-arginine;
(d) 0.62 mg/ml calcium chloride; and
(e) 1.12 mg/ml poloxamer 188.

In some embodiments, disclosed herein is a pharmaceutical composition according to any of the disclosed embodiments, which further comprises a pH of about 6.5 to about 7.5. In some embodiments, the pharmaceutical composition disclosed herein has a pH of about 7.0. In some embodiments, the pharmaceutical composition disclosed herein has a pH of about 6.8. In some embodiments the pharmaceutical composition disclosed herein does not comprise NaCl. In some embodiments the pharmaceutical composition disclosed herein comprises less than 8.8 mg/mL sodium chloride (NaCl). In some embodiments, the pharmaceutical composition disclosed herein comprises L-histidine. In some embodiments, the pharmaceutical composition disclosed herein comprises L-arginine. In some embodiments, the pharmaceutical composition disclosed herein comprises arginine-HCl. In some embodiments, the pharmaceutical composition disclosed herein comprises L-arginine-HCl. In some embodiments, the pharmaceutical composition disclosed herein comprises calcium chloride dihydrate.

In some embodiments, disclosed herein is a pharmaceutical composition according to any of the disclosed embodiments, wherein the pharmaceutical composition has a chimeric protein concentration of about 0.8 to about 1.2 mg/mL. In some embodiments, the pharmaceutical composition disclosed herein comprises 75 IU/mL to 2,000 IU/ml of the chimeric protein. In some embodiments, the pharmaceutical composition disclosed herein has an osmolality about 525 to about 725 mOsm/kg. In some embodiments, the pharmaceutical composition disclosed herein has an osmolality about 500 to about 650 mOsm/kg. In some embodiments, the pharmaceutical composition disclosed herein has an osmolality about 600 to about 650 mOsm/kg. In some embodiments, the pharmaceutical composition disclosed herein has a turbidity of less than about 7 Nephelometric Turbidity Units (NTU).

In some embodiments, disclosed herein is a method of storing a pharmaceutical composition, comprising maintaining a pharmaceutical composition disclosed herein at a temperature of from about −20° C. to about −40° C. for at least about 1 to about 36 months. In some embodiments, the pharmaceutical composition is stored at a temperature of about −20° C., about −25° C., about −30° C., about −35° C., or about −40° C. for at least about 1 to about 36 months. In some embodiments, the pharmaceutical composition is stored at a temperature of about −20° C., about −25° C., about −30° C., about −35° C., or about −40° C. for at least about 3, 6, 9, 12, 18, 21, 24, 27, 30, 33, or 36 months. In some embodiments, the pharmaceutical composition is stored at a temperature of about −30° C. for at least about 1, 2, 3, 4, 5, 6, 7, 8, 9, 11, or 12 months.

In some embodiments, the pharmaceutical composition has been stored at a temperature of from about −20° C. to about −40° C. for at least about 1 to about 36 months. In some embodiments, the pharmaceutical composition has been stored at a temperature of about −20° C., about −25° C., about −30° C., about −35° C., or about −40° C. for at least about 1 to about 36 months. In some embodiments, the pharmaceutical composition has been stored at a temperature of about −20° C., about −25° C., about −30° C., about −35° C., or about −40° C. for at least about 3, 6, 9, 12, 18, 21, 24, 27, 30, 33, or 36 months. In some embodiments, the pharmaceutical composition has been stored at a temperature of about −30° C. for at least about 1, 2, 3, 4, 5, 6, 7, 8, 9, 11, or 12 months.

In some embodiments, the pharmaceutical composition is a liquid pharmaceutical composition that has been frozen and then thawed 2-5 times. In some embodiments, the pharmaceutical composition is a liquid pharmaceutical composition that has been frozen and then thawed 2 times. In some embodiments, the pharmaceutical composition is a liquid pharmaceutical composition that has been frozen and then thawed 3 times. In some embodiments, the pharmaceutical composition is a liquid pharmaceutical composition that has been frozen and then thawed 4 times. In some embodiments, the pharmaceutical composition is a liquid pharmaceutical composition that has been frozen and then thawed 5 times.

In some embodiments, the pharmaceutical composition comprises about 0.5 mg/ml to about 10 mg/ml of the chimeric protein. In some embodiments, the pharmaceutical composition comprises about 0.5 mg/ml to about 5 mg/ml of the chimeric protein. In some embodiments, the pharmaceutical composition comprises about 0.5 mg/ml to about 2 mg/ml of the chimeric protein. In some embodiments, the pharmaceutical composition comprises about 0.5 mg/ml of the chimeric protein. In some embodiments, the pharmaceutical composition comprises about 1 mg/ml of the chimeric protein. In some embodiments, the pharmaceutical composition comprises about 1.5 mg/ml of the chimeric protein. In some embodiments, the pharmaceutical composition comprises about 2 mg/ml of the chimeric protein.

Also disclosed herein is a method of treating hemophilia A in a subject in need thereof, comprising administering to the subject an effective amount of the pharmaceutical composition according to any of the embodiments disclosed herein. In some embodiments, the pharmaceutical composition is self-administered. In some embodiments, the pharmaceutical composition is administered intravenously. In some embodiments, the pharmaceutical composition is administered intravenously at a dose of about 20 IU/kg to about 70 IU/kg. In some embodiments, the pharmaceutical composition is administered intravenously at a dose of about 50 IU/kg. In some embodiments, the pharmaceutical composition is administered intravenously once every 7-10 days. In some embodiments, the pharmaceutical composition is administered intravenously once weekly.

Also disclosed herein is a pharmaceutical kit comprising (i) a first container comprising a lyophilized pharmaceutical composition comprising
(a) a chimeric protein comprising a first polypeptide chain which comprises a Factor VIII (“FVIII”) protein or a portion thereof and a first immunoglobulin (“Ig”) constant region or a portion thereof, and a second polypeptide chain which comprises a von Willebrand Factor (“VWF”) protein and a second Ig constant region or a portion thereof;
(b) about 10 mg to about 200 mg sucrose;
(c) about 2.5 mg to about 7.5 mg histidine;
(d) about 140 mg to about 200 mg arginine;
(e) about 1.5 mg to about 5 mg calcium chloride; and
(f) about 1 mg to about 10 mg poloxamer 188, and
(ii) a second container comprising sterile water.

In some embodiments, the chimeric protein comprises a first polypeptide chain comprising the amino acid sequence set forth as SEQ ID NO: 1 and a second polypeptide chain comprising the amino acid sequence set forth as SEQ ID NO: 2, wherein the first polypeptide chain and the second polypeptide chain are covalently linked by two disulfide bonds between Fc domains in the first and second polypeptide chains.

In some embodiments, the pharmaceutical kit disclosed herein comprises a lyophilized pharmaceutical composition comprising:
(a) about 10 mg to about 200 mg sucrose;
(b) about 2.5 mg to about 7.5 mg histidine;
(c) about 140 mg to about 200 mg arginine;
(d) about 1.5 mg to about 5 mg calcium chloride; and
(e) about 1 mg to about 10 mg poloxamer 188.

In some embodiments the pharmaceutical kit comprises a lyophilized pharmaceutical composition that does not comprise NaCl. In some embodiments the lyophilized pharmaceutical composition comprises less than 8.8 mg/mL sodium chloride (NaCl). In some embodiments, the lyophilized pharmaceutical composition comprises L-histidine. In some embodiments, the lyophilized pharmaceutical composition comprises L-arginine. In some embodiments, the lyophilized pharmaceutical composition comprises arginine-HCl.

In some embodiments, the lyophilized pharmaceutical composition comprises L-arginine-HCl. In some embodiments, the lyophilized pharmaceutical composition disclosed herein comprises calcium chloride dihydrate.

In some embodiments, the pharmaceutical kit comprises a lyophilized pharmaceutical composition comprising:
(a) about 33.7 mg sucrose;
(b) about 5.2 mg L-histidine;
(c) about 177.3 mg L-arginine-HCl;
(d) about 2.5 mg calcium chloride; and
(e) about 3.4 mg poloxamer 188.

In some embodiments, the pharmaceutical kit comprises a lyophilized pharmaceutical composition comprising:
(a) about 67.3 mg sucrose;
(b) about 5.2 mg L-histidine;
(c) about 177.3 mg L-arginine-HCl;
(d) about 2.5 mg calcium chloride; and
(e) about 3.4 mg poloxamer 188.

In some embodiments, the pharmaceutical kit comprises a lyophilized pharmaceutical composition comprising:
(a) about 67.3 mg sucrose;
(b) about 5.2 mg L-histidine;
(c) about 146.6 mg L-arginine-HCl;
(d) about 2.5 mg calcium chloride; and
(e) about 3.4 mg poloxamer 188.

In some embodiments, the pharmaceutical kit comprises a lyophilized pharmaceutical composition comprising:
(a) about 168.3 mg sucrose;
(b) about 5.2 mg L-histidine;
(c) about 177.3 mg L-arginine-HCl;
(d) about 2.5 mg calcium chloride; and
(e) about 3.4 mg poloxamer 188.

In some embodiments, the pharmaceutical kit comprises a lyophilized pharmaceutical composition having a moisture content of less than 2%. In some embodiments, the lyophilized pharmaceutical composition has a moisture content of less than 1.8%. In some embodiments, the lyophilized pharmaceutical composition has a moisture content of less than 1.6%. In some embodiments, the lyophilized pharmaceutical composition is in a lyophilized cake. In some embodiments, the lyophilized cake is white. In some embodiments, the lyophilized cake is less than Y4 in the European Pharmacopoeia color scale. In some embodiments, the lyophilized pharmaceutical composition is a powder.

In some embodiments, the pharmaceutical kit comprises a first container comprising 100 IU to 10,000 IU of the chimeric protein. In some embodiments, the first container comprises 250 IU, 500 IU, 1000 IU, 2000 IU, 3000 IU, or 4,000 IU of the chimeric protein. In some embodiments, the first container comprises more than about 250 IU of the chimeric protein. In some embodiments, the first container comprises more than about 300 IU of the chimeric protein. In some embodiments, the first container comprises more than about 500 IU of the chimeric protein. In some embodiments, the first container comprises at least about 500 IU of the chimeric protein.

In some embodiments, the pharmaceutical kit further comprises instructions for combining the lyophilized pharmaceutical composition and sterile water. In some embodiments, when the lyophilized pharmaceutical composition and the sterile water are combined, then the lyophilized pharmaceutical composition is reconstituted within 7 to 12 seconds.

In some embodiments, the pharmaceutical composition comprises a second container comprising sterilized water at a volume sufficient to produce, when combined with the lyophilized powder of the first container, a solution comprising

In some embodiments, when the lyophilized pharmaceutical composition and the sterile water are combined, then the osmolality of the resulting solution is about 525 to about 725 mOsm/kg. In some embodiments, the pharmaceutical composition disclosed herein has an osmolality about 500 to about 650 mOsm/kg. In some embodiments, the osmolality of the resulting solution is about 600 to about 650 mOsm/kg. In some embodiments, the pH of the resulting solution is about 6.5 to about 7.5. In some embodiments, the pH of the resulting solution is about 7.0. In some embodiments, the pH of the resulting solution is about 6.8. In some embodiments, the the turbidity of the resulting solution is less than about 7 Nephelometric Turbidity Units (NTU). In some embodiments, the protein concentration of the resulting solution is about 0.8 to about 1.2 mg/ml. In some embodiments, less than 3% of the protein is aggregated.

In some embodiments, the pharmaceutical kit comprises a second container comprising sterilized water at a volume sufficient to produce, when combined with the lyophilized pharmaceutical composition of the first container, a solution comprising:
(a) 10 mg/mL to 60 mg/mL sucrose;
(b) 1.5 mg/mL to 2.0 mg/mL L-histidine;
(c) 40 mg/mL to 70 mg/mL L-arginine-HCl;
(d) 0.5 mg/mL to 0.9 mg/mL calcium chloride; and
(e) 0.6 mg/mL to 1.6 mg/mL poloxamer 188.

In some embodiments of the pharmaceutical kit disclosed herein, when the lyophilized pharmaceutical composition and the sterile water are combined, then the resulting solution comprises:
(a) 10 mg/mL to 60 mg/mL sucrose;
(b) 1.5 mg/mL to 2.0 mg/mL L-histidine;
(c) 40 mg/mL to 70 mg/mL L-arginine-HCl;
(d) 0.5 mg/mL to 0.9 mg/mL calcium chloride; and
(e) 0.6 mg/mL to 1.6 mg/mL poloxamer 188.

In some embodiments of the pharmaceutical kit disclosed herein, when the lyophilized pharmaceutical composition and the sterile water are combined, then the resulting solution comprises:
(a) 11.23 mg/ml sucrose;
(b) 1.74 mg/ml L-histidine;
(c) 59.11 mg/ml L-arginine-HCl;
(d) 0.62 mg/ml calcium chloride; and
(e) 1.12 mg/ml poloxamer 188.

In some embodiments of the pharmaceutical kit disclosed herein, when the lyophilized pharmaceutical composition and the sterile water are combined, then the resulting solution comprises:
(a) 22.45 mg/ml sucrose;
(b) 1.74 mg/ml L-histidine;
(c) 59.11 mg/ml L-arginine-HCl;
(d) 0.62 mg/ml calcium chloride; and
(e) 1.12 mg/ml poloxamer 188.

In some embodiments of the pharmaceutical kit disclosed herein, when the lyophilized pharmaceutical composition and the sterile water are combined, then the resulting solution comprises:
(a) 22.45 mg/ml sucrose;
(b) 1.74 mg/ml L-histidine;
(c) 48.88 mg/ml L-arginine;
(d) 0.62 mg/ml calcium chloride; and
(e) 1.12 mg/ml poloxamer 188.

In some embodiments of the pharmaceutical kit disclosed herein, when the lyophilized pharmaceutical composition and the sterile water are combined, then the resulting solution comprises:
(a) 56.12 mg/ml sucrose;
(b) 1.74 mg/ml L-histidine;
(c) 59.11 mg/ml L-arginine-HCl;
(d) 0.62 mg/ml calcium chloride; and
(e) 1.12 mg/ml poloxamer 188.

In some embodiments, the pharmaceutical kit comprises a second container comprising about 2 mL to about 5 mL of sterile water. In some embodiments, the pharmaceutical kit comprises a second container comprising about 3 mL of sterile water. In some embodiments, the pharmaceutical kit comprises a second container comprising about 3.3 mL of sterile water.

In some embodiments, the pharmaceutical kit is stored at a temperature of from about −20° C. to about −40° C. for at least about 1 to about 36 months. In some embodiments, the pharmaceutical kit is stored at a temperature of about −20° C., about −25° C., about-30° C., about −35° C., or about −40° C. for at least about 1 to about 36 months. In some embodiments, the pharmaceutical kit is stored at a temperature of about −20° C., about −25° C., about −30° C., about −35° C., or about −40° C. for at least about 3, 6, 9, 12, 18, 21, 24, 27, 30, 33, or 36 months. In some embodiments, the pharmaceutical kit is stored at a temperature of about −30° C. for at least about 1, 2, 3, 4, 5, 6, 7, 8, 9, 11, or 12 months.

In some embodiments, the pharmaceutical kit has been stored at a temperature of from about −20° C. to about −40° C. for at least about 1 to about 36 months. In some embodiments, the pharmaceutical kit has been stored at a temperature of about −20° C., about −25° C., about −30° C., about −35° C., or about −40° C. for at least about 1 to about 36 months. In some embodiments, the pharmaceutical kit has been stored at a temperature of about −20° C., about −25° C., about −30° C., about −35° C., or about −40° C. for at least about 3, 6, 9, 12, 18, 21, 24, 27, 30, 33, or 36 months. In some embodiments, the pharmaceutical kit has been stored at a temperature of about −30° C. for at least about 1, 2, 3, 4, 5, 6, 7, 8, 9, 11, or 12 months.

In some embodiments, the pharmaceutical kit comprises a first container which is a glass vial comprising a rubber stopper. In some embodiments, the pharmaceutical kit comprises a second container which is a syringe body. In some embodiments, the sterile water is in the syringe body. In some embodiments, the syringe body is associated with a plunger. In some embodiments, the pharmaceutical kit further comprises an adaptor to connect the glass vial to the syringe body. In some embodiments, the pharmaceutical kit further comprises infusion tubing associated with a needle to be connected to the syringe body, suitable for intravenous infusion.

Also disclosed herein is a method of treating hemophilia A in a subject in need thereof, comprising combining the lyophilized pharmaceutical composition and the sterile water of the pharmaceutical kit according to any of the embodiments disclosed herein, and administering to the subject an effective amount of the resulting combination (i.e. solution). In some embodiments, the subject combines the lyophilized pharmaceutical composition and the sterile water of the kit. In some embodiments, the combination is self-administered by the subject.

### BRIEF DESCRIPTION OF THE DRAWINGS

FIG. 1 is a schematic representation of efanesoctocog alfa, an exemplary FVIII-ELNN-Fc/D′D3-ELNN-Fc heterodimer. FVIII: factor VIII; VWF: von Willebrand Factor; A1, A2, A3, C1, C2: domains of FVIII; D′D3: domains of VWF; Fc: Fc region of immunoglobulin constant region.

FIG. 2 shows the aggregation levels (% HMWS) over time (hours) at room temperature (RT)/room light (RL) conditions for efanesoctocog alfa drug substance (DS) compositions (1 mg/mL) containing either 0.05% (w/v) PS80 or 0.1% (w/v) poloxamer 188 as a surfactant and either 1% or 5% (w/v) sucrose. Rates of change in aggregation levels (slope) are also provided (% per hour)

FIG. 3 shows the aggregation levels (% HMWS) over time (hours) at 2-8° C. for efanesoctocog alfa drug substance (DS) compositions (1 mg/mL) containing either 0.05% (w/v) PS80 or 0.1% (w/v) poloxamer 188 as a surfactant and either 1% or 5% (w/V) sucrose. Rates of change in aggregation levels (slope) are also provided (% per hour).

FIG. 4 shows the aggregation levels (% HMWS) of efanesoctocog alfa drug substance (DS) compositions flowed by dilution to 4000 IU bulk drug product at 7 hours and containing either 0.05% (w/v) PS80 or 0.1% (w/v) poloxamer 188 as a surfactant and 1% or 5% (w/V) sucrose. Samples were held at RT and tested after 0, 5, 7, 25, 43, and 55 hours. Samples were also tested after lyophilization (Post-Lyo).

FIG. 5 shows a frozen storage stability study which measured the aggregation levels (% HMWS) of efanesoctocog alfa DS compositions (1 mg/mL) containing 0.05% (w/V) PS80 as a surfactant. Compositions containing 0, 1, 2, or 5% (w/v) sucrose were held at freezing temperatures (−80° C. or −30° C.). Time points measured were pre-freeze, start of experiment (TO), one month (T1M), 3 months (T3M), and 6 months (T6M).

FIG. 6 shows a frozen storage stability study which measured the aggregation levels (% HMWS) of efanesoctocog alfa DS compositions (1 mg/mL) containing 0.1% (w/v) poloxamer 188 as a surfactant. Compositions containing 0, 1, 2, or 5% (w/V) sucrose were held at freezing temperatures (−80° C. or −30° C.). Time points measured are pre-freeze, start of experiment (TO), one month (T1M), 3 months (T3M), and 6 months (T6M).

FIGS. 7A-7B show the results of an analysis of efanesoctocog alfa DS compositions (1 mg/mL) by particle testing using high accuracy liquid particle counter (HIAC). DS compositions containing 0.1% (w/v) poloxamer 188 and 0, 1, 2, or 5% (w/v) sucrose were held at −80° C. and assessed at the following time points: pre-freeze, start of experiment (TO), one month (T1M), 3 months (T3M), and 6 months (T6M). Results for ≥10 μm particles are shown in FIG. 7A. Results for ≥25 μm particles are shown in FIG. 7B.

FIGS. 8A-8C show the aggregation levels (% HMWS) for compositions of efanesoctocog alfa lyophilized drug product (Lyo DP) at 250 IU and comprising 0.1% (w/v) poloxamer 188 as surfactant. Lyo DP compositions containing 1% or 5% (w/v) sucrose were tested. Samples were held at 5° C., 30° C., or 40° C. and tested at TO, 1 month, 2 months, 3 months, and 6 months. Results are shown in FIG. 8A (5° C.), FIG. 8B (30° C.), and FIG. 8C (40° C.).

FIGS. 9A-9C show the aggregation levels (% HMWS) for compositions of efanesoctocog alfa lyophilized drug product (Lyo DP) at 4000 IU and comprising 0.1% (w/v) poloxamer 188 as surfactant. Lyo DP compositions containing 1% or 5% (w/v) sucrose were tested. Samples were held at 5° C., 30° C., or 40° C. and tested at TO, 1 month, 2 months, 3 months, and 6 months. Results are shown in FIG. 9A (5° C.), FIG. 9B (30° C.), and FIG. 9C (40° C.).

FIG. 10 shows the aggregation levels (% HMWS) of compositions of efanesoctocog alfa DS (1 mg/mL) before (pre freeze) and after stress by freeze/thaw (F/T) at either −80° C. or −30° C. for 5 cycles (5×) (not less than 24 hrs, thaw at room temperature). FIG. 10 shows the results for DS compositions containing 0.05% (w/v) PS80 and 0, 1, 2, or 5% (w/v) sucrose.

FIG. 11 shows the aggregation levels (% HMWS) of compositions of efanesoctocog alfa DS (1 mg/mL) before (pre freeze) and after stress by freeze/thaw (F/T) at either-80° C. or −30° C. for 5 cycles (5×) (not less than 24 hrs, thaw at room temperature). FIG. 11 shows the results for DS compositions containing 0.1% (w/v) poloxamer 188 and 0, 1, 2, or 5% (w/v) sucrose.

FIGS. 12A-12B show protein concentration analysis by A280 (absorbance at 280 nm) of different sucrose concentrations in efanesoctocog alfa DS (1 mg/mL) compositions following F/T stress. Samples were analyzed before (pre freeze) and after 1, 3, or 5 F/T cycles. Results for DS compositions containing 0.05% (w/v) PS80 and 0, 1, 2, or 5% (w/V) sucrose are shown in FIG. 12A. Results for DS compositions containing 0.1% (w/V) poloxamer 188 and 0, 1, 2, or 5% (w/v) sucrose are shown in FIG. 12B.

FIG. 13 shows an analysis of pH following F/T stress for efanesoctocog alfa DS compositions containing either 0.05% (w/v) PS80 or 0.1% (w/v) poloxamer 188 as a surfactant and 0, 1, 2, or 5% (w/v) sucrose. pH was measured after 0, 1, 3, or 5 F/T cycles.

FIG. 14 shows the glass transition temperature (Tg) and residual moisture content (%) at the start of experiment (TO) for compositions of efanesoctocog alfa lyophilized drug product (Lyo DP) at 250 IU. Lyo DP compositions containing either 0.05% (w/v) PS80 or 0.1% (w/v) poloxamer 188 and 0, 1, 2, or 5% (w/v) sucrose were tested.

FIG. 15 shows the glass transition temperature (Tg) and residual moisture content (%) at the start of experiment (TO) for compositions of efanesoctocog alfa lyophilized drug product (Lyo DP) at 4000 IU. Lyo DP compositions containing either 0.05% (w/v) PS80 or 0.1% (w/v) poloxamer 188 and 0, 1, 2, or 5% (w/v) sucrose were tested.

FIGS. 16A-16C show the glass transition temperature (Tg) of efanesoctocog alfa lyophilized drug product (Lyo DP) compositions at 4000 IU or 250 IU and containing 0.1% (w/v) poloxamer 188 as a surfactant and either 5% or 1% (w/v) sucrose. Samples were held at 5° C., 30° C., or 40° C. and tested at TO, 1 month, 2 months, 3 months, and 6 months. Results are shown in FIG. 16A (5° C.), FIG. 16B (30° C.), and FIG. 16C (40° C.).

FIG. 17 shows the glass transition temperature (Tg) of efanesoctocog alfa liquid bulk drug product (BDP) compositions at 250 IU or 4000 IU and containing either 0.05% (w/v) PS80 or 0.1% (w/v) poloxamer 188 as a surfactant and 0, 1, 2, or 5% (w/v) sucrose.

FIGS. 18A-18C show the residual moisture content (%) of compositions of efanesoctocog alfa lyophilized drug product (Lyo DP) at 4000 IU or 250 IU. Lyo DP compositions containing 0.1% (w/v) poloxamer 188 as surfactant and 1% or 5% (w/v) sucrose were tested. Samples were held at 5° C., 30° C., or 40° C. and tested at TO, 1 month, 2 months, 3 months, and 6 months. Results are shown in FIG. 18A (5° C.), FIG. 18B (30° C.), and FIG. 18C (40° C.).

FIG. 19 shows the aggregation levels (% HMWS) after stress by freeze/thaw (F/T) for compositions of efanesoctocog alfa DS (concentration: >2 mg/mL) comprising increasing concentrations of arginine (Arg). Compositions contained Arginine concentrations tested were 100, 125, 150, 175, 200, and 250 mM. Each column represents repeat injections from the same vial after thawing for 30 minutes at RT. Time points measured were immediately post-thaw and 40, 80, 120, and 160 minutes post-thaw.

FIG. 20 shows the mean concentration (ng/mL) of efanesoctocog alfa over time as determined by ELISA following a single IV bolus injection of a composition comprising either PS80 or P188. Vehicle 1: 10 mM L-Histidine, 250 mM Arginine-HCl, 5 mM calcium chloride, 5% w/v Sucrose, 0.05% Polysorbate-80. Vehicle 2: 10 mM L-Histidine, 250 mM Arginine-HCl, 5 mM Calcium Chloride, 5% w/v Sucrose, 0.1% Poloxamer-188.

FIG. 21 shows the mean FVIII activity (mIU/mL) of efanesoctocog alfa over time as determined by chromogenic assay following a single IV bolus injection of a composition comprising either PS80 or P188. Vehicle 1: 10 mM L-Histidine, 250 mM Arginine-HCl, 5 mM Calcium Chloride, 5% w/v Sucrose, 0.05% Polysorbate 80. Vehicle 2: 10 mM L-Histidine, 250 mM Arginine-HCl, 5 mM Calcium Chloride, 5% w/v Sucrose, 0.1% Poloxamer 188.

### DETAILED DESCRIPTION OF THE DISCLOSURE

The present disclosure is directed to, inter alia, formulations (including aqueous and lyophilized formulations, as well as related kits) comprising FVIII proteins. In some embodiments, the FVIII protein is a chimeric FVIII protein such as efanesoctocog alfacomprising two polypeptides, i.e., a first polypeptide comprising a FVIII protein comprising a first ELNN Polypeptide sequence insert fused to a first Fc region, and a second polypeptide comprising a VWF protein fused to a second Ig constant region by a second ELNN Polypeptide sequence, wherein the first ELNN Polypeptide sequence contains about 288 amino acids and the second ELNN Polypeptide sequence contains about 144 amino acids, and the first Ig constant region and the second Ig constant region are covalently linked together by disulfide bonds.

The present disclosure is provides to formulations (including aqueous and lyophilized formulations, as well as related kits) for a chimeric protein chimeric protein comprising (i) a factor VIII (FVIII) polypeptide and (ii) a von Willebrand factor (VWF) fragment comprising a D′ domain of VWF and a D3 domain of VWF. Included herein are compositions that may be lyophilized, as well as compositions formed upon reconstitution of lyophilized formulations with a diluent. Therapeutic methods and uses are also provided.

### I. Definitions

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, may provide one of skill with a general dictionary of many of the terms used in this disclosure.

Units, prefixes, and symbols are denoted in their Système International de Unites (SI) accepted form. Numeric ranges are inclusive of the numbers defining the range. Unless otherwise indicated, amino acid sequences are written left to right in amino to carboxy orientation. The headings provided herein are not limitations of the various aspects of the disclosure. Accordingly, the terms defined immediately below are more fully defined by reference to the specification in its entirety.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower). In some embodiments, the term indicates deviation from the indicated numerical value by ±10%, ±5%, ±4%, ±3%, ±2%, ±1%, ±0.9%, ±0.8%, ±0.7%, ±0.6%, ±0.5%, ±0.4%, ±0.3%, ±0.2%, ±0.1%, ±0.05%, or ±0.01%. In some embodiments, “about” indicates deviation from the indicated numerical value by ±10%. In some embodiments, “about” indicates deviation from the indicated numerical value by ±5%. In some embodiments, “about” indicates deviation from the indicated numerical value by ±4%. In some embodiments, “about” indicates deviation from the indicated numerical value by ±3%. In some embodiments, “about” indicates deviation from the indicated numerical value by ±2%. In some embodiments, “about” indicates deviation from the indicated numerical value by ±1%. In some embodiments, “about” indicates deviation from the indicated numerical value by ±0.9%. In some embodiments, “about” indicates deviation from the indicated numerical value by ±0.8%. In some embodiments, “about” indicates deviation from the indicated numerical value by ±0.7%. In some embodiments, “about” indicates deviation from the indicated numerical value by ±0.6%. In some embodiments, “about” indicates deviation from the indicated numerical value by ±0.5%. In some embodiments, “about” indicates deviation from the indicated numerical value by ±0.4%. In some embodiments, “about” indicates deviation from the indicated numerical value by ±0.3%. In some embodiments, “about” indicates deviation from the indicated numerical value by ±0.1%. In some embodiments, “about” indicates deviation from the indicated numerical value by ±0.05%. In some embodiments, “about” indicates deviation from the indicated numerical value by ±0.01%.

Depending on context, the term “polynucleotide” or “nucleotide” may encompass a singular nucleic acid as well as plural nucleic acids. In some embodiments, a polynucleotide is an isolated nucleic acid molecule or construct, e.g., messenger RNA (mRNA) or plasmid DNA (pDNA). In some embodiments, a polynucleotide comprises a conventional phosphodiester bond. In some embodiments, a polynucleotide comprises a non-conventional bond (e.g., an amide bond, such as found in peptide nucleic acids (PNA)). The term “nucleic acid” may refer to any one or more nucleic acid segments, e.g., DNA or RNA fragments, present in a polynucleotide. By “isolated” nucleic acid or polynucleotide is intended a nucleic acid molecule, DNA or RNA, which has been removed from its native environment. For example, a recombinant polynucleotide encoding a Factor VIII polypeptide contained in a vector is considered isolated for the purposes of the present disclosure. Further examples of an isolated polynucleotide include recombinant polynucleotides maintained in heterologous host cells or purified (partially or substantially) from other polynucleotides in a solution. Isolated RNA molecules include in vivo or in vitro RNA transcripts of polynucleotides of the present disclosure. Isolated polynucleotides or nucleic acids according to the present disclosure further include such molecules produced synthetically. In addition, a polynucleotide or a nucleic acid can include regulatory elements such as promoters, enhancers, ribosome binding sites, or transcription termination signals.

Certain proteins secreted by mammalian cells are associated with a secretory signal peptide which is cleaved from the mature protein once export of the growing protein chain across the rough endoplasmic reticulum has been initiated. Those of ordinary skill in the art are aware that signal peptides are generally fused to the N-terminus of the polypeptide, and are cleaved from the complete or “full-length” polypeptide to produce a secreted or “mature” form of the polypeptide. In some embodiments, a native signal peptide or a functional derivative of that sequence that retains the ability to direct the secretion of the polypeptide that is operably associated with it. Alternatively, a heterologous mammalian signal peptide, e.g., a human tissue plasminogen activator (TPA) or mouse β-glucuronidase signal peptide, or a functional derivative thereof, can be used.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” can be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide can be derived from a natural biological source or produced recombinant technology, but is not necessarily translated from a designated nucleic acid sequence. It can be generated in any manner, including by chemical synthesis.

An “isolated” polypeptide or a fragment, variant, or derivative thereof refers to a polypeptide that is not in its natural milieu. No particular level of purification is required. For example, an isolated polypeptide can simply be removed from its native or natural environment. Recombinantly produced polypeptides and proteins expressed in host cells are considered isolated for the purpose of the disclosure, as are native or recombinant polypeptides which have been separated, fractionated, or partially or substantially purified by any suitable technique.

Also included in the present disclosure are fragments or variants of polypeptides, and any combination thereof. The term “fragment” or “variant” when referring to polypeptide binding domains or binding molecules of the present disclosure include any polypeptides which retain at least some of the properties (e.g., FcRn binding affinity for an FcRn binding domain or Fc variant, coagulation activity for an FVIII variant, or FVIII binding activity for the VWF fragment) of the reference polypeptide. Fragments of polypeptides include proteolytic fragments, as well as deletion fragments, in addition to specific antibody fragments discussed elsewhere herein, but do not include the naturally occurring full-length polypeptide (or mature polypeptide). Variants of polypeptide binding domains or binding molecules of the present disclosure include fragments as described above, and also polypeptides with altered amino acid sequences due to amino acid substitutions, deletions, or insertions. Variants can be naturally or non-naturally occurring. Non-naturally occurring variants can be produced using art-known mutagenesis techniques. Variant polypeptides can comprise conservative or non-conservative amino acid substitutions, deletions or additions.

The term “VWF protein” as used herein means any VWF fragment that interacts with FVIII and retains at least one or more properties that are normally provided to FVIII by full-length VWF, e.g., preventing premature activation to FVIIIa, preventing premature proteolysis, preventing clearance, preventing association with phospholipid membranes that could lead to premature clearance, preventing binding to FVIII clearance receptors that can bind naked FVIII but not VWF-bound FVIII, and/or stabilizing the FVIII heavy chain and light chain interactions. A VWF fragment referred to herein is a VWF polypeptide that is less than the full-length VWF protein, wherein the VWF fragment retains the ability to interact with and/or bind to FVIII. In some embodiments, a VWF protein is a fragment (which may be mutated) of full-length VWF that binds to a FVIII protein such that the FVIII protein has reduced binding to, or does not bind, full length VWF (e.g., endogenous VWF in a subject).

A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, if an amino acid in a polypeptide is replaced with another amino acid from the same side chain family, the substitution is considered to be conservative. In some embodiments, a string of amino acids can be conservatively replaced with a structurally similar string that differs in order and/or composition of side chain family members.

As known in the art, “sequence identity” between two polypeptides is determined by comparing the amino acid sequence of one polypeptide to the sequence of a second polypeptide. Similarly, “sequence identity” between two polynucleotides is determined by comparing the nucleotide sequence of one polynucleotide to the sequence of a second polynucleotide. The terms “% identical”, “% identity” or similar terms are intended to refer, in particular, to the percentage of nucleotides or amino acids (as applicable) which are identical in an optimal alignment between the sequences to be compared. Said percentage is purely statistical, and the differences between the two sequences may be but are not necessarily randomly distributed over the entire length of the sequences to be compared. Comparisons of two sequences are usually carried out by comparing the sequences, after optimal alignment, with respect to a segment or “window of comparison”, in order to identify local regions of corresponding sequences. For example, the optimal alignment for a comparison may be carried out manually or with the aid of the local homology algorithm by Smith and Waterman, 1981, Ads App. Math. 2, 482, with the aid of the local homology algorithm by Neddleman and Wunsch, 1970, J. Mol. Biol. 48, 443, with the aid of the similarity search algorithm by Pearson and Lipman, 1988, Proc. Natl Acad. Sci. USA 88, 2444, or with the aid of computer programs using said algorithms (GAP, BESTFIT, FASTA, BLAST P, BLAST N and TFASTA in Wisconsin Genetics Software Package, Genetics Computer Group, 575 Science Drive, Madison, Wis.). In some embodiments, percent identity of two sequences is determined using the BLASTN or BLASTP algorithm, as available on the United States National Center for Biotechnology Information (NCBI) website (e.g, at blast.ncbi.nlm.nih.gov/Blast.cgi?PAGE_TYPE=BlastSearch&BLAST_SPEC=blast2seq&L INK_LOC=align2seq). In some embodiments, the algorithm parameters used for BLASTN algorithm on the NCBI website include: (i) Expect Threshold set to 10; (ii) Word Size set to 28; (iii) Max matches in a query range set to 0; (iv) Match/Mismatch Scores set to 1, −2; (v) Gap Costs set to Linear; and (vi) the filter for low complexity regions being used. In some embodiments, the algorithm parameters used for BLASTP algorithm on the NCBI website include: (i) Expect Threshold set to 10; (ii) Word Size set to 3; (iii) Max matches in a query range set to 0; (iv) Matrix set to BLOSUM62; (v) Gap Costs set to Existence: 11 Extension: 1; and (vi) conditional compositional score matrix adjustment. When discussed herein, whether any particular polypeptide is at least about 50%, 60%, 70%, 75%, 80%, 85%, 90%, 95%, 99%, or 100% identical to another polypeptide can be determined using methods and computer programs/software known in the art such as, but not limited to, the BESTFIT program (Wisconsin Sequence Analysis Package, Version 8 for Unix, Genetics Computer Group, University Research Park, 575 Science Drive, Madison, WI 53711). BESTFIT uses the local homology algorithm of Smith and Waterman, Advances in Applied Mathematics 2:482-489 (1981), to find the best segment of homology between two sequences. When using BESTFIT or any other sequence alignment program to determine whether a particular sequence is, for example, 95% identical to a reference sequence according to the present disclosure, the parameters are set, of course, such that the percentage of identity is calculated over the full-length of the reference polypeptide sequence and that gaps in homology of up to 5% of the total number of amino acids in the reference sequence are allowed.

As used herein, an “amino acid corresponding to” or an “equivalent amino acid” in a VWF sequence or a FVIII protein sequence is identified by alignment to maximize the identity or similarity between a first VWF or FVIII sequence and a second VWF or FVIII sequence. The number used to identify an equivalent amino acid in a second VWF or FVIII sequence is based on the number used to identify the corresponding amino acid in the first VWF or FVIII sequence.

As used herein, the term “insertion site” refers to a position in a FVIII polypeptide, or fragment, variant, or derivative thereof, which is immediately downstream of the position at which a half-life extending moiety or heterologous moiety can be inserted. An “insertion site” is specified as a number, the number being the number of the amino acid in mature native FVIII (SEQ ID NO: 9) to which the insertion site corresponds, which is immediately C-terminal to the position of the insertion. For example, the phrase “comprises an ELNN Polypeptide at an insertion site which corresponds to amino acid 1656 of SEQ ID NO: 9” indicates that the heterologous moiety is located between two amino acids corresponding to amino acid 1656 and amino acid 1657 of SEQ ID NO: 9.

The phrase “immediately downstream of an amino acid” as used herein refers to position right next to the terminal carboxyl group of the amino acid. For example, an insertion site immediately downstream of amino acid 745 corresponding to the mature wild type FVIII protein (SEQ ID NO: 9) means that the insertion site is between amino acid 745 and amino acid 746 corresponding to the mature wild type FVIII protein. Similarly, the phrase “immediately upstream of an amino acid” refers to the position right next to the terminal amine group of the amino acid.

The phrase “between two amino acids of an insertion site” as used herein refers to a position in which an ELNN Polypeptide or any other polypeptide is inserted between two adjacent amino acids. Thus, the phrases “inserted immediately downstream of an amino acid” and “inserted between two amino acids of an insertion site” are used synonymously with “inserted at an insertion site.”

The terms “inserted,” “is inserted,” “inserted into” or grammatically related terms, as used herein with respect to insertions of ELNN Polypeptide into FVIII refers to the position of an ELNN Polypeptide in a chimeric protein relative to the analogous position in native mature human FVIII. As used herein the terms refer to the characteristics of the recombinant FVIII polypeptide relative to native mature human FVIII, and do not indicate, imply or infer any methods or process by which the chimeric protein was made. For example, in reference to a chimeric protein provided herein, the phrase “an ELNN Polypeptide is inserted immediately downstream of residue 745 of the FVIII polypeptide” means that the chimeric protein comprises an ELNN Polypeptide immediately downstream of an amino acid which corresponds to amino acid 745 in native mature human FVIII, e.g., bounded by amino acids corresponding to amino acids 745 and 746 of native mature human FVIII (without requiring the presence of an amino acid corresponding to 746 of native mature human FVIII), and does not connote an order or method of production for which the chimeric protein was constructed.

As used herein, the terms “ELNN Polypeptide” and “ELNN” are synonymous, and refer to extended length polypeptides with non-naturally occurring, substantially non-repetitive sequences that are composed mainly of small hydrophilic amino acids, with the sequence having a low degree or no secondary or tertiary structure under physiologic conditions. ELNNs can confer certain desirable pharmacokinetic, physicochemical and pharmaceutical properties when linked to a VWF protein or a FVIII sequence of the disclosure to create a chimeric polypeptide. Such desirable properties include but are not limited to enhanced pharmacokinetic parameters and solubility characteristics. As used herein, the terms “ELNN Polypeptide” and “ELNN” specifically exclude antibodies or antibody fragments such as single-chain antibodies or Fc fragments of a light chain or a heavy chain. ELNN polypeptides are known in the art, and non-limiting descriptions relating to and examples of ELNN polypeptides known as XTEN® polypeptides are available in Schellenberger et al., (2009) Nat Biotechnol 27 (12): 1186-90; Brandl et al., (2020) Journal of Controlled Release 327:186-197; and Radon et al., (2021) Advanced Functional Materials 31, 2101633 (pages 1-33), the entire contents of each of which are incorporated herein by reference.

A “fusion” or “chimeric” protein comprises a first amino acid sequence linked to a second amino acid sequence with which it is not naturally linked in nature. The amino acid sequences which normally exist in separate proteins can be brought together in the fusion polypeptide, or the amino acid sequences which normally exist in the same protein can be placed in a new arrangement in the fusion polypeptide, e.g., fusion of a Factor VIII domain of the disclosure with an Ig Fc domain. A fusion protein is created, for example, by chemical synthesis, or by creating and translating a polynucleotide in which the peptide regions are encoded in the desired relationship. A chimeric protein can further comprise a second amino acid sequence associated with the first amino acid sequence by a covalent, non-peptide bond or a non-covalent bond.

With respect to sequences, the term “linked” as used herein refers to a first amino acid sequence or nucleotide sequence covalently or non-covalently joined to a second amino acid sequence or nucleotide sequence, respectively. The first amino acid or nucleotide sequence can be directly joined or juxtaposed to the second amino acid or nucleotide sequence or alternatively an intervening sequence can covalently join the first sequence to the second sequence. Depending on context, the term “linked” means not only a fusion of a first amino acid sequence to a second amino acid sequence at the C-terminus or the N-terminus, but also includes insertion of the whole first amino acid sequence (or the second amino acid sequence) into any two amino acids in the second amino acid sequence (or the first amino acid sequence, respectively). In some embodiments, the first amino acid sequence can be linked to a second amino acid sequence by a peptide bond or a linker. The first nucleotide sequence can be linked to a second nucleotide sequence by a phosphodiester bond or a linker. The linker can be a peptide or a polypeptide (for polypeptide chains) or a nucleotide or a nucleotide chain (for nucleotide chains) or any chemical moiety (for both polypeptide and polynucleotide chains). The term “linked” may also be indicated by a hyphen (-).

With respect to two polypeptides, the term “associated with” refers to one or more covalent or non-covalent bonds formed between a first polypeptide and a second polypeptide. In some embodiments, the term “associated with” means a covalent, non-peptide bond or a non-covalent bond. This association can be indicated by a colon, i.e., (:). In some embodiments, it means a covalent bond except a peptide bond. For example, the amino acid cysteine comprises a thiol group that can form a disulfide bond or bridge with a thiol group on a second cysteine residue. In most naturally occurring IgG molecules, the CH1 and CL regions are associated by a disulfide bond and the two heavy chains are associated by two disulfide bonds at positions corresponding to 239 and 242 using the Kabat numbering system (position 226 or 229, EU numbering system). Examples of covalent bonds include, but are not limited to, a peptide bond, a metal bond, a hydrogen bond, a disulfide bond, a sigma bond, a pi bond, a delta bond, a glycosidic bond, an agnostic bond, a bent bond, a dipolar bond, a Pi backbond, a double bond, a triple bond, a quadruple bond, a quintuple bond, a sextuple bond, conjugation, hyperconjugation, aromaticity, hapticity, or antibonding. Non-limiting examples of non-covalent bond include an ionic bond (e.g., cation-pi bond or salt bond), a metal bond, a hydrogen bond (e.g., dihydrogen bond, dihydrogen complex, low-barrier hydrogen bond, or symmetric hydrogen bond), van der Walls force, London dispersion force, a mechanical bond, a halogen bond, aurophilicity, intercalation, stacking, entropic force, or chemical polarity. In some embodiments, the one or more covalent bonds between the first amino acid chain and the second amino acid chain is two disulfide bonds. In some embodiments, the one or more covalent bonds between the first amino acid chain and the second amino acid chain is two disulfide bonds between a first Fc portion on the first amino acid chain and a second Fc portion on the second amino acid chain, wherein the two disulfide bonds occur in the hinge region of the two Fc portions.

In some embodiments, a polypeptide has an enzymatic cleavage site cleaved by an enzyme that is activated during the clotting cascade, such that cleavage of such sites occurs at the site of clot formation. Exemplary such sites include, e.g., those recognized by thrombin, Factor XIa or Factor Xa. SEQ ID NO: 9SEQ ID NO: 15Other enzymatic cleavage sites are known in the art and described in elsewhere herein. In constructs that include more than one processing or cleavage site, it will be understood that such sites can be the same or different. As used herein, the term “half-life” refers to a biological half-life of a particular polypeptide in vivo. Half-life can be represented by the time required for half the quantity administered to a subject to be cleared from the circulation and/or other tissues in the animal. In some embodiments, when a clearance curve of a given polypeptide is constructed as a function of time, the curve is usually biphasic with a rapid α-phase and longer β-phase. The α-phase typically represents an equilibration of the administered Fc polypeptide between the intra- and extra-vascular space and is, in part, determined by the size of the polypeptide. The β-phase typically represents the catabolism of the polypeptide in the intravascular space. In some embodiments, FVIII and chimeric proteins comprising FVIII are monophasic, and thus do not have an alpha phase, but just the single beta phase. Therefore, in some embodiments, the term half-life as used herein refers to the half-life of the polypeptide in the β-phase. The typical beta phase half-life of a human antibody in humans is 21 days. In some embodiments, the half-life is expressed as the half-life of the terminal phase.

In some embodiments, a subject has hemophilia A. In some embodiments, the hemophilia A is severe hemophilia A.

“Administer” or “administering,” as used herein refers to delivering to a subject a composition described herein, e.g., a chimeric protein. The composition, e.g., the chimeric protein, can be administered to a subject using methods known in the art. In particular, the composition can be administered intravenously, subcutaneously, intramuscularly, intradermally, or via any mucosal surface, e.g., orally, sublingually, buccally, nasally, rectally, vaginally or via pulmonary route. In some embodiments, the administration is intravenous. In some embodiments, the administration is subcutaneous. In some embodiments, the administration is self-administration. In some embodiments, a parent administers the chimeric protein to a child. In some embodiments, the chimeric protein is administered to a subject by a healthcare practitioner such as a medical doctor, a medic, or a nurse.

As used herein, the term “dose” refers to a single administration of a composition to a subject. A single dose can be administered all at once, e.g., as a bullous, or over a period of time, e.g., via an intravenous infusion. The term “multiple doses” means more than one dose, e.g., more than one administration.

When referring to co-administration of more than one composition, a dose of composition A can be administered concurrently with a dose of composition B. Alternatively, a dose of composition A can be administered before or after a dose of composition B. In some embodiments, composition A and composition B are combined into a single formulation.

As used herein, the term “interval” or “dosing interval” refers to the amount of time that elapses between a first dose of composition A and a subsequent dose of the same composition administered to a subject. A dosing interval can refer to the time that elapses between a first dose and a second dose, or a dosing interval can refer to the amount of time that elapses between multiple doses.

The term “dosing frequency” as used herein refers to the number of doses administered per a specific dosing interval. For example, a dosing frequency can be written as once a week, once every two weeks, etc. Therefore, a dosing interval of 7 days can be also written as a dosing interval of once in 7 days or once every week, or once a week.

As used herein the term “prophylactic treatment” refers to the administration of a therapy for the treatment of hemophilia A, where such treatment is intended to prevent or reduce the severity of one or more symptoms of hemophilia A, e.g., bleeding episodes, e.g., one or more spontaneous bleeding episodes, and/or joint damage. See Jimenez-Yuste et al., Blood Transfus. 12 (3): 314-19 (2014). To prevent or reduce the severity of such symptoms, e.g., bleeding episodes and the progression of joint disease, hemophilia A patients may receive regular infusions of clotting factor as part of a prophylactic treatment regimen. The basis of such prophylactic treatment is the observation that hemophilia patients with a clotting factor, e.g., FVIII, level of 1% or more rarely experience spontaneous bleeding episodes and have fewer hemophilia-related comorbidities as compared to patients with severe hemophilia. See, e.g., Coppola A. et al, Semin. Thromb. Hemost. 38 (1): 79-94 (2012). Health care practitioners treating these hemophilia patients surmised that maintaining factor levels at around 1% with regular infusions could potentially reduce the risk of hemophilia symptoms, including bleeding episodes and joint damage. See id. Subsequent research has confirmed these benefits in pediatric hemophilia patients receiving prophylactic treatment with clotting factor, rendering prophylactic treatment the goal for people with severe hemophilia. See id.

A “prophylactic” treatment can also refer to the preemptive administration of the composition described herein, e.g., a protein (such as a chimeric protein), to a subject in order to control, manage, prevent, or reduce the occurrence or severity of one or more symptoms of hemophilia A, e.g., bleeding episodes. In some embodiments, prophylactic treatment with a clotting factor, e.g., FVIII, is used to treat subjects with severe hemophilia A. In some embodiments, prophylactic treatment refers to administering a composition disclosed herein to a subject in need thereof to reduce the occurrence of one or more symptom of hemophilia A. A prophylactic treatment can include administration of multiple doses. The multiple doses used in prophylactic treatment are typically administered at particular dosing intervals. In some embodiments, the annualized bleeding rate can be reduced to less than 10, less than 9, less than 8, less than 7, less than 6, less than 5, less than 4, less than 3, less than 2, or less than 1.

The term “on-demand treatment” or “episodic treatment” refers to the “as needed” administration of a chimeric molecule in response to symptoms of hemophilia A, e.g., a bleeding episode, or before an activity that can cause bleeding. In some aspects, the on-demand treatment can be given to a subject when bleeding starts, such as after an injury, or when bleeding is expected, such as before surgery. In some aspects, the on-demand treatment can be given prior to activities that increase the risk of bleeding, such as contact sports. In some embodiments, the on-demand treatment is given as a single dose. In some embodiments, the on-demand treatment is given as a first dose, followed by one or more additional doses. When the chimeric protein is administered on-demand, the one or more additional doses can be administered at least about 12 hours, at least about 24 hours, at least about 36 hours, at least about 48 hours, at least about 60 hours, at least about 72 hours, at least about 84 hours, at least about 96 hours, at least about 108 hours, or at least about 120 hours after the first dose. It should be noted, however, that the dosing interval associated with on-demand treatment is not the same as the dosing interval used for prophylactic treatment.

In some embodiments, the subject in need of a general hemostatic agent is undergoing, or is about to undergo, surgery. The chimeric protein of the disclosure can be administered prior to or after surgery. The chimeric protein of the disclosure can also be administered during or after surgery to control an acute bleeding episode. When the chimeric protein is administered prior to surgery, the administration can be at least about 1 hour, at least about 2 hours, at least about 4 hours, at least about 8 hours, at least about 12 hours, at least about 24 hours, at least about 36 hours, at least about 48 hours, or at least about 72 hours prior to surgery. When the chimeric protein is administered to after surgery, the administration can be at least about 1 hour, at least about 2 hours, at least about 4 hours, at least about 8 hours, at least about 12 hours, at least about 24 hours, at least about 36 hours, at least about 48 hours, or at least about 72 hours after surgery. The surgery can include, but is not limited to, liver transplantation, liver resection, dental procedures, or stem cell transplantation.

“Treat”, “treatment”, “treating”, as used herein refers to, e.g., the reduction in severity of a disease or condition; the reduction in the duration of a disease course; the amelioration of one or more symptoms associated with a disease or condition; the provision of beneficial effects to a subject with a disease or condition, without necessarily curing the disease or condition, or the prophylaxis of one or more symptoms associated with a disease or condition. In some embodiments, treating or treatment comprises maintaining a FVIII trough level at least about 1 IU/dL, 2 IU/dL, 3 IU/dL, 4 IU/dL, 5 IU/dL, 6 IU/dL, 7 IU/dL, 8 IU/dL, 9 IU/dL, 10 IU/dL, 11 IU/dL, 12 IU/dL, 13 IU/dL, 14 IU/dL, 15 IU/dL, 16 IU/dL, 17 IU/dL, 18 IU/dL, 19 IU/dL, or 20 IU/dL in a subject by administering a chimeric protein of the disclosure. As used herein, a “trough level” in a hemophilia A patient is the measurement of the lowest concentration reached by a factor therapy, e.g., a FVIII therapy, before the next dose is administered. In some embodiments, treating or treatment means maintaining a FVIII trough level of at least about 1 IU/dL between the dosing interval. In some embodiments, treating or treatment means maintaining a FVIII trough level of at least about 3 IU/dL between the dosing interval. In some embodiments, treating or treatment means maintaining a FVIII trough level of at least about 5 IU/dL between the dosing interval. In some embodiments, treating or treatment means maintaining a FVIII trough level between about 1 and about 20 IU/dL, about 2 and about 20 IU/dL, about 3 and about 20 IU/dL, about 4 and about 20 IU/dL, about 5 and about 20 IU/dL, about 6 and about 20 IU/dL, about 7 and about 20 IU/dL, about 8 and about 20 IU/dL, about 9 and about 20 IU/dL, or about 10 and about 20 IU/dL during the dosing interval.

In some embodiments, treatment or treating of a disease or condition comprises maintaining FVIII activity in a subject at a level comparable to at least about 1%, 2%, 3%, 4%, 5%, 6%, 7%, 8%, 9%, 10%, 11%, 12%, 13%, 14%, 15%, 16%, 17%, 18%, 19%, or 20% of the FVIII activity in a non-hemophiliac subject between the dosing interval. In some embodiments, treating or treatment means maintaining a FVIII activity level of at least about 1% between the dosing interval. In some embodiments, treating or treatment means maintaining a FVIII activity level of at least about 2% between the dosing interval. In some embodiments, treating or treatment means maintaining a FVIII activity level of at least about 3% between the dosing interval. In some embodiments, treating or treatment means maintaining a FVIII activity level of at least about 4% between the dosing interval. In some embodiments, treating or treatment means maintaining a FVIII activity level of at least about 5%. between the dosing interval. In some embodiments, treating or treatment means maintaining a FVIII activity level of at least about 6% between the dosing interval. In some embodiments, treating or treatment means maintaining a FVIII activity level of at least about 7% between the dosing interval. In some embodiments, treating or treatment means maintaining a FVIII activity level of at least about 8% between the dosing interval. In some embodiments, treating or treatment means maintaining a FVIII activity level of at least about 9% between the dosing interval. In some embodiments, treating or treatment means maintaining a FVIII activity level of at least about 10% between the dosing interval. In some embodiments, the minimum trough level required for treatment can be measured by one or more known methods (for example, the activated partial thromboplastin time (aPTT) assays or chromogenic assays, which are well known in the art) and can be adjusted (increased or decreased) for each person. Non-limiting examples of assays for measuring trough level are disclosed in U.S. Application Publication No. 20190375822, which is hereby incorporated by reference in its entirety.

### II. Chimeric Proteins

In an aspect, the present disclosure is directed to pharmaceutical compositions comprising a chimeric protein or protein which comprises a first polypeptide chain which comprises a Factor VIII (“FVIII”) protein or a portion thereof and a first immunoglobulin (“Ig”) constant region or a portion thereof, and a second polypeptide chain which comprises a von Willebrand Factor (“VWF”) protein and a second Ig constant region or a portion thereof. In some embodiments, the chimeric protein comprises (i) a FVIII protein comprising a FVIII polypeptide, an ELNN Polypeptide inserted within the B domain of the FVIII polypeptide, and a first Fc region; and (ii) a VWF protein comprising a VWF fragment, a second ELNN Polypeptide sequence, an a2 linker, and a second Fc region. In some embodiments, the chimeric protein disclosed herein is a FVIII-ELNN-Fc/D′D3-ELNN-Fc heterodimer.

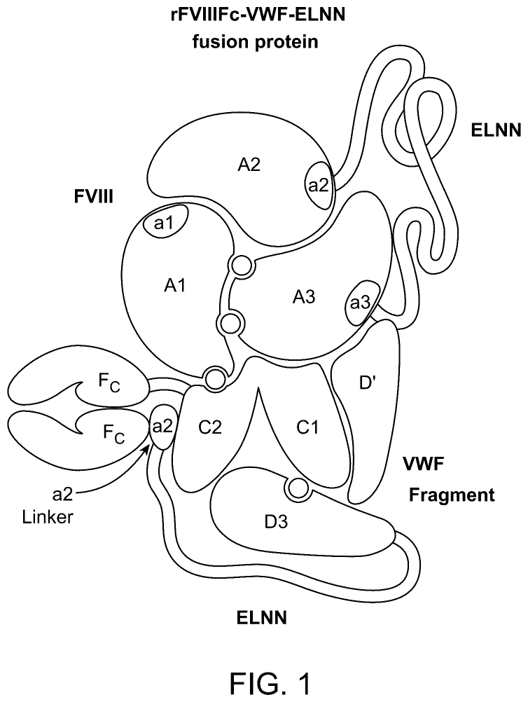

In some embodiments, the chimeric protein is efanesoctocog alfa. Efanesoctocog alfa, also known as “BIVV001”, “efanesoctocogum alfa”, “rFVIIIFc-VWF-ELNN”, and “rFVIIIFc-VWF-XTEN”, is described in Chhabra et al. Blood 135 (17): 1484-1496 (2020), the entire contents of which are hereby incorporated by reference in its entirety. A schematic representation of efanesoctocog alfa, as an exemplary FVIII-ELNN-Fc/D′D3-ELNN-Fc heterodimer, is presented in FIG. 1.

Efanesoctocog alfa is a large protein (over 300 kDa) comprising multiple parts on each of two polypeptide chains that are associated by covalent bonds and non-covalent interactions. The protein has a tendency to aggregate under certain conditions, which can reduce the stability of formulations unless an excipient such as L-arginine is selected and present in an amount that is sufficient to reduce the aggregation. For example, the aggregation may be reduced by adding high levels of L-arginine (e.g., about 250 mM).

Additional information regarding efanesoctocog alfa can be found in the International Nonproprietary Names for Pharmaceutical Substances (INN) WHO Drug Information, 2019, Vol. 33, No. 4, p. 828-30. In some embodiments, the chimeric protein is a FVIII-ELNN-Fc/D′D3-ELNN-Fc heterodimer comprising (i) a first polypeptide comprising the amino acid sequence of SEQ ID NO: 1 and (ii) a second polypeptide comprising the amino acid sequence of SEQ ID NO: 2. In some embodiments, the chimeric protein comprises (i) a first polypeptide and (ii) a second polypeptide that are covalently linked via one or more disulfide bonds (e.g., two disulfide bonds). In some embodiments, the chimeric protein comprises a FVIII protein encoded by the nucleic acid sequence of SEQ ID NO: 4. In some embodiments, the chimeric protein comprises a VWF protein encoded by the nucleic acid sequence of SEQ ID NO: 6. In some embodiments, the efanesoctocog alfa has an activity of at least 1600 IU/mg. In some embodiments, the efanesoctocog alfa has an activity of at least 1700 IU/mg. In some embodiments, the efanesoctocog alfa has an activity of at least 1800 IU/mg. In some embodiments, the efanesoctocog alfa has an activity of at least 1900 IU/mg. In some embodiments, the efanesoctocog alfa has an activity of 1600 IU/mg to 2000 IU/mg.

In some embodiments, the chimeric protein comprises a FVIII protein comprising the amino acid sequence of SEQ ID NO: 1. In some embodiments, the chimeric protein comprises a FVIII protein comprising one or more disulfide bridges at one or more of the following locations: residues 153-179, 248-329, 528-554, 630-711, 1220-1246, 1287-1291, 1409-1557, 1562-1714, 1761-1821, and/or 1867-1925 of SEQ ID NO: 1. In some embodiments, the chimeric protein comprises a FVIII protein comprising one or more disulfide bridges at each of the following locations: residues 153-179, 248-329, 528-554, 630-711, 1220-1246, 1287-1291, 1409-1557, 1562-1714, 1761-1821, and 1867-1925 of SEQ ID NO: 1. In some embodiments, the chimeric protein comprises a FVIII protein comprising one or more Cys-SH residues at residues 310, 692, and/or 1388 of SEQ ID NO: 1. In some embodiments, the chimeric protein comprises a FVIII protein comprising a Cys-SH residues at each of residues 310, 692, and/or 1388 of SEQ ID NO: 1.

In some embodiments, the chimeric protein comprises a FVIII protein comprises one or more N-glycosylation sites at residues N41, N239, N1198, N1506, and/or N1797 of SEQ ID NO: 1. In some embodiments, the chimeric protein comprises a FVIII protein comprises one or more O-glycosylation sites at residues 746-1036 of SEQ ID NO: 1 and/or the Ser and Thr residues in the linker peptides. In some embodiments, the chimeric protein comprises a FVIII protein comprises one or more Tyr-sulfation sites at residues 346, 718, 719, 723, 729, 1052, and/or 1068 of SEQ ID NO: 1.

In some embodiments, the chimeric protein comprises a VWF protein comprising the amino acid sequence of SEQ ID NO: 2. In some embodiments, the chimeric protein comprises a VWF protein comprising one or more disulfide bridges at one or more of the following locations: residues 4-45, 13-41, 25-36, 29-64, 47-58, 66-88, 83-100, 86-95, 104-233, 126-268, 135-230, 151-158, 283-326, 297-321, 308-348, 328-334, 338-363, 367-410, 386-406, 390-402, 394-433, 414-427, 436-464, 459-474, 462-471, 698-758, and/or 804-862 of SEQ ID NO: 2. In some embodiments, the chimeric protein comprises a VWF protein comprising one or more disulfide bridges at each of the following locations: residues 4-45, 13-41, 25-36, 29-64, 47-58, 66-88, 83-100, 86-95, 104-233, 126-268, 135-230, 151-158, 283-326, 297-321, 308-348, 328-334, 338-363, 367-410, 386-406, 390-402, 394-433, 414-427, 436-464, 459-474, 462-471, 698-758, and/or 804-862 of SEQ ID NO: 2.

In some embodiments, the chimeric protein comprises a VWF protein comprises one or more N-glycosylation sites at residues N94, N384, N734 of SEQ ID NO: 2. In some embodiments, the chimeric protein comprises a VWF protein comprises one or more O-glycosylation sites at residues 478-625 of SEQ ID NO: 2 and/or the Ser and Thr residues in the linker peptides. In some embodiments, the chimeric protein comprises a VWF protein comprises one or more Tyr-sulfation sites at residues 632, 633, 637, and/or 643 of SEQ ID NO: 2. In some embodiments, the VWF protein comprises a VWF fragment comprising a D1, D2, D′, and/or D3 domain of VWF. In one embodiment, the VWF fragment comprises a D1D2 region of VWF comprising the amino acid sequence of SEQ ID NO: 20. In some embodiments, the VWF protein further comprises a VWF signal peptide sequence. In one embodiment, the VWF signal peptide comprises the amino acid sequence of SEQ ID NO: 19. In one specific embodiment, the VWF protein comprises a VWF signal peptide comprising the amino acid sequence of SEQ ID NO: 19, a D1D2 region of VWF comprising the amino acid sequence of SEQ ID NO: 20, a D′ domain of VWF comprising the amino acid sequence of SEQ ID NO: 21, a D3 domain of VWF comprising the amino acid sequence of SEQ ID NO: 22, an ELNN Polypeptide sequence comprising the amino acid sequence of SEQ ID NO: 14 (AE144_5A), an a2 linker comprising the amino acid sequence of SEQ ID NO: 15, and/or a Fc region comprising the amino acid sequence of SEQ ID NO: 23.

In some embodiments, the chimeric protein of the present disclosure comprises: (i) a FVIII protein comprising a FVIII polypeptide, a first ELNN Polypeptide sequence, and a first Fc region; and (ii) a VWF fragment comprising a D′ domain of VWF and a D3 domain of VWF, a second ELNN Polypeptide sequence, an a2 linker of FVIII, and a second Fc region; wherein: the FVIII protein has a deletion of amino acids 746 to 1648 corresponding to mature FVIII; the first ELNN Polypeptide sequence is inserted within the FVIII polypeptide immediately downstream of amino acid 745 corresponding to mature FVIII; the first ELNN Polypeptide sequence comprises an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to the amino acid sequence of AE288 (SEQ ID NO: 9); the first Fc region is fused to the C-terminus of the FVIII polypeptide; the second ELNN Polypeptide sequence is fused to the C-terminus of the VWF fragment; the second ELNN Polypeptide sequence comprises an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to the amino acid sequence of AE144_5A (SEQ ID NO: 14); the a2 linker is fused to the C-terminus of the ELNN Polypeptide; the a2 linker comprises an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to the amino acid sequence of SEQ ID NO: 9; the second Fc region is fused to the C-terminus of the a2 linker; and the first Fc region is covalently linked to the second Fc region by a disulfide bond (e.g., two disulfide bonds).

In some embodiments, the chimeric protein of the disclosure comprises two polypeptide sequences, a first polypeptide sequence comprising an amino acid sequence at least about 80%, 90%, 95%, or 100% identical to the amino acid sequence set forth in SEQ ID NO: 1; and a second polypeptide sequence comprising a VWF fragment comprising a D′ domain of VWF and a D3 domain of VWF and an Fc region. In some embodiments, the chimeric protein of the disclosure comprises two polypeptide sequences, a first polypeptide sequence comprising FVIII polypeptide and an Fc region; and a second polypeptide sequence comprising an amino acid sequence at least about 80%, 90%, 95%, or 100% identical to the amino acid sequence set forth in SEQ ID NO: 2. In some embodiments, the chimeric protein of the disclosure comprises two polypeptide sequences, a first polypeptide sequence comprising an amino acid sequence at least about 80%, 90%, 95%, or 100% identical to the amino acid sequence set forth in SEQ ID NO: 1 and a second polypeptide sequence comprising an amino acid sequence at least about 80%, 90%, 95%, or 100% identical to the amino acid sequence set forth in SEQ ID NO: 2. In some embodiments, the chimeric protein of the disclosure comprises two polypeptide sequences, a first polypeptide sequence comprising the amino acid sequence set forth in SEQ ID NO: 7 and a second polypeptide sequence comprising the amino acid sequence set forth in SEQ ID NO: 2. In some embodiments, the chimeric protein of the disclosure comprises two polypeptide sequences, a first polypeptide sequence comprising the amino acid sequence set forth in SEQ ID NO: 1 and a second polypeptide sequence comprising the amino acid sequence set forth in SEQ ID NO: 2, wherein the first polypeptide sequence and the second polypeptide sequence are linked to each other by a disulfide bond. In some embodiments, the chimeric protein of the disclosure comprises two polypeptide sequences, a first polypeptide sequence comprising the amino acid sequence set forth in SEQ ID NO: 1 and a second polypeptide sequence comprising the amino acid sequence set forth in SEQ ID NO: 2, wherein the first polypeptide sequence and the second polypeptide sequence are linked to each other by two disulfide bonds. In some embodiments, the chimeric protein of the disclosure comprises two polypeptide sequences, a first polypeptide sequence comprising the amino acid sequence set forth in SEQ ID NO: 1 and a second polypeptide sequence comprising the amino acid sequence set forth in SEQ ID NO: 2, wherein the first polypeptide sequence comprises a first Fc portion, wherein the second polypeptide sequence comprises a second Fc portion, wherein the first Fc portion and the second Fc portion are linked to each other by two disulfide bonds in the hinge region.

In some embodiments, the chimeric protein of the disclosure comprises a FVIII protein comprising an amino acid sequence at least about 80%, 90%, 95%, or 100% identical to SEQ ID NO: 7, SEQ ID NO: 3, or SEQ ID NO: 1; and a VWF protein comprising an amino acid sequence at least about 80%, 90%, 95%, or 100% identical to SEQ ID NO: 2 or SEQ ID NO: 5.

In some embodiments, the chimeric protein of the disclosure comprises: (i) a FVIII protein comprising a first FVIII polypeptide fragment comprising the amino acid sequence of SEQ ID NO: 17; a first ELNN Polypeptide sequence comprising the amino acid sequence of SEQ ID NO: 9 (AE288); a second FVIII polypeptide fragment comprising the amino acid sequence of SEQ ID NO: 18; and a first Fc region comprising the amino acid sequence of SEQ ID NO: 23; and (ii) a VWF protein comprising: a D′ domain of VWF comprising the amino acid sequence of SEQ ID NO: 21; a D3 domain of VWF comprising the amino acid sequence of SEQ ID NO: 22; a second ELNN Polypeptide sequence comprising the amino acid sequence of SEQ ID NO: 14 (AE144_5A); an a2 linker comprising the amino acid sequence of SEQ ID NO: 15; and a second Fc region comprising the amino acid sequence of SEQ ID NO: 23, and wherein the first Fc region is covalently linked to the second Fc region by a disulfide bond (e.g., two disulfide bonds).

In some embodiments, the chimeric protein of the disclosure comprises a FVIII protein comprising a FVIII polypeptide, a first ELNN Polypeptide sequence, a first Fc region, and a VWF protein comprising a D′ domain of VWF, a D3 domain of VWF, a second ELNN Polypeptide sequence, an a2 linker of FVIII and a second Fc region, wherein the FVIII polypeptide comprises the amino acid sequence of SEQ ID NO: 17, the first ELNN Polypeptide sequence comprises the amino acid sequence of AE288 (SEQ ID NO: 9) and is fused to the C-terminus of SEQ ID NO: 17, the FVIII polypeptide further comprises the amino acid sequence of SEQ ID NO: 18, the first Fc region comprises the amino acid sequence of SEQ ID NO: 23 and is fused to the C-terminus of SEQ ID NO: 18; the D′ domain of VWF comprises the amino acid sequence of SEQ ID NO: 21; the D3 domain of VWF comprises the amino acid sequence of SEQ ID NO: 214, the second ELNN Polypeptide sequence comprises the amino acid sequence of AE144_5A (SEQ ID NO: 14) and is fused to the C-terminus of the D3 domain of VWF; the a2 linker comprises the amino acid sequence of SEQ ID NO: 15 and is fused to the C-terminus of the second ELNN Polypeptide sequence; the second Fc region comprises the amino acid sequence of SEQ ID NO: 23 and is fused to the C-terminus of the a2 linker; and wherein the first Fc region is covalently linked to the second Fc region by a disulfide bond.

In some embodiments, the chimeric protein of the disclosure comprises a FVIII protein comprising a FVIII signal peptide comprising the amino acid sequence of SEQ ID NO: 16. In some embodiments, the chimeric protein comprises a VWF protein comprising a VWF signal peptide comprising the amino acid sequence of SEQ ID NO: 19. In some embodiments, the chimeric protein comprises a VWF protein comprising a D1D2 domain of VWF comprising the amino acid sequence of SEQ ID NO: 20.

In some embodiments, the chimeric protein comprises a first polypeptide comprising the amino acid sequence of SEQ ID NO: 3 and a second polypeptide comprising the amino acid sequence of SEQ ID NO: 5. In some embodiments, the chimeric protein comprises a first polypeptide comprising the amino acid sequence of SEQ ID NO: 7 and a second polypeptide comprising the amino acid sequence of SEQ ID NO: 2. In some embodiments, the chimeric protein comprises a first polypeptide comprising the amino acid sequence of SEQ ID NO: 1 and a second polypeptide comprising the amino acid sequence of SEQ ID NO: 2.

In some embodiments, the chimeric protein comprises one or more disulfide bridges between the first polypeptide and the second polypeptide. In some embodiments, the chimeric protein comprises two disulfide bridges between the first polypeptide and the second polypeptide. In some embodiments, the chimeric protein comprises a first polypeptide comprising the amino acid sequence of SEQ ID NO: 1 and a second polypeptide comprising the amino acid sequence of SEQ ID NO: 2, wherein the chimeric protein comprises a disulfide bridge between residue 1726 of SEQ ID NO: 1 and residue 663 of SEQ ID NO: 2, and a disulfide bridge between residue 1729 of SEQ ID NO: 1 and residue 666 of SEQ ID NO: 2.

### IV. Pharmaceutical Compositions

In an aspect, the present disclosure is directed to pharmaceutical compositions of a chimeric protein which are formulated to improve protein stability. In some embodiments, the disclosed pharmaceutical compositions demonstrate increased stability based on analysis by visual inspection, protein concentration, pH stability, formation of high molecular weight species (HMWS), and/or change in turbidity. Analysis of these properties of stability can be made using conventional techniques, including size exclusion chromatography (SEC), reversed-phase high-performance liquid chromatography (RP-HPLC), and many others.

The pharmaceutical compositions disclosed herein comprise a specified amount of the chimeric protein. In some embodiments, the pharmaceutical composition has a chimeric protein concentration of about 0.8 to about 1.2 mg/mL. In some embodiments, the pharmaceutical composition has a chimeric protein concentration of about 0.8 mg/mL. In some embodiments, the pharmaceutical composition has a chimeric protein concentration of about 0.9 mg/mL. In some embodiments, the pharmaceutical composition has a chimeric protein concentration of about 1.0 mg/mL. In some embodiments, the pharmaceutical composition has a chimeric protein concentration of about 1.1 mg/mL. In some embodiments, the pharmaceutical composition has a chimeric protein concentration of about 1.2 mg/mL.

In some embodiments, the pharmaceutical composition comprises about 75 IU/mL to about 2,000 IU/mL of the chimeric protein. In some embodiments, the pharmaceutical composition comprises about 75 IU/mL of the chimeric protein. In some embodiments, the pharmaceutical composition comprises about 100 IU/mL of the chimeric protein. In some embodiments, the pharmaceutical composition comprises about 150 IU/mL of the chimeric protein. In some embodiments, the pharmaceutical composition comprises about 200 IU/mL of the chimeric protein. In some embodiments, the pharmaceutical composition comprises about 250 IU/mL of the chimeric protein. In some embodiments, the pharmaceutical composition comprises about 300 IU/mL of the chimeric protein. In some embodiments, the pharmaceutical composition comprises about 350 IU/mL of the chimeric protein. In some embodiments, the pharmaceutical composition comprises about 400 IU/mL of the chimeric protein. In some embodiments, the pharmaceutical composition comprises about 450 IU/mL of the chimeric protein. In some embodiments, the pharmaceutical composition comprises about 500 IU/mL of the chimeric protein. In some embodiments, the pharmaceutical composition comprises about 550 IU/mL of the chimeric protein. In some embodiments, the pharmaceutical composition comprises about 600 IU/mL of the chimeric protein. In some embodiments, the pharmaceutical composition comprises about 650 IU/mL of the chimeric protein. In some embodiments, the pharmaceutical composition comprises about 700 IU/mL of the chimeric protein. In some embodiments, the pharmaceutical composition comprises about 750 IU/mL of the chimeric protein. In some embodiments, the pharmaceutical composition comprises about 800 IU/mL of the chimeric protein. In some embodiments, the pharmaceutical composition comprises about 850 IU/mL of the chimeric protein. In some embodiments, the pharmaceutical composition comprises about 900 IU/mL of the chimeric protein. In some embodiments, the pharmaceutical composition comprises about 950 IU/mL of the chimeric protein. In some embodiments, the pharmaceutical composition comprises about 1000 IU/mL of the chimeric protein. In some embodiments, the pharmaceutical composition comprises more than about 250 IU of the chimeric protein. In some embodiments, the pharmaceutical composition comprises more than about 300 IU of the chimeric protein. In some embodiments, the pharmaceutical composition comprises more than about 500 IU of the chimeric protein. In some embodiments, the pharmaceutical composition comprises at least about 500 IU of the chimeric protein. In some embodiments, the pharmaceutical composition comprises more than about 85 IU/ml of the chimeric protein. In some embodiments, the pharmaceutical composition comprises more than about 100 IU/ml of the chimeric protein. In some embodiments, the pharmaceutical composition comprises more than about 200 IU/ml of the chimeric protein. In some embodiments, the pharmaceutical composition comprises at least about 200 IU/ml of the chimeric protein.

In some embodiments, the pharmaceutical composition comprises about 1100 IU/mL of the chimeric protein. In some embodiments, the pharmaceutical composition comprises about 1150 IU/mL of the chimeric protein. In some embodiments, the pharmaceutical composition comprises about 1200 IU/mL of the chimeric protein. In some embodiments, the pharmaceutical composition comprises about 1250 IU/mL of the chimeric protein. In some embodiments, the pharmaceutical composition comprises about 1300 IU/mL of the chimeric protein. In some embodiments, the pharmaceutical composition comprises about 1350 IU/mL of the chimeric protein. In some embodiments, the pharmaceutical composition comprises about 1400 IU/mL of the chimeric protein. In some embodiments, the pharmaceutical composition comprises about 1450 IU/mL of the chimeric protein. In some embodiments, the pharmaceutical composition comprises about 1500 IU/mL of the chimeric protein. In some embodiments, the pharmaceutical composition comprises about 1550 IU/mL of the chimeric protein. In some embodiments, the pharmaceutical composition comprises about 1600 IU/mL of the chimeric protein. In some embodiments, the pharmaceutical composition comprises about 1650 IU/mL of the chimeric protein. In some embodiments, the pharmaceutical composition comprises about 1700 IU/mL of the chimeric protein. In some embodiments, the pharmaceutical composition comprises about 1750 IU/mL of the chimeric protein. In some embodiments, the pharmaceutical composition comprises about 1800 IU/mL of the chimeric protein. In some embodiments, the pharmaceutical composition comprises about 1850 IU/mL of the chimeric protein. In some embodiments, the pharmaceutical composition comprises about 1900 IU/mL of the chimeric protein. In some embodiments, the pharmaceutical composition comprises about 1950 IU/mL of the chimeric protein. In some embodiments, the pharmaceutical composition comprises about 2000 IU/mL of the chimeric protein.

Pharmaceutical compositions containing the chimeric protein of the present disclosure also contain a suitable pharmaceutically acceptable carrier. For example, they can contain excipients and/or auxiliaries that provide enhanced stability of the chimeric protein or facilitate processing of the active compounds into preparations designed for delivery to the site of action.

In an aspect, disclosed herein are pharmaceutical compositions comprising a specified amount of a chimeric protein along with excipients as disclosed. The pharmaceutical compositions disclosed herein comprise various concentrations of these excipients as disclosed, and the concentrations can be expressed in various ways. For example, the concentration of a given excipient can be expressed as a molar concentration (e.g., M or mM), as a weight/volume percent, (e.g., grams per 100 ml diluent), or as milligrams per milliliter (mg/ml). Pharmaceutical compositions provided herein can contain specified amounts of the various excipients at a level of precision ranging from approximate, e.g., concentrations expressed only to one significant figure (e.g., about 0.1% (w/v)), or with more precision, e.g., out to 2, 3, 4, 5, or 6 significant figures (e.g., about 3.88 mg/ml, with precision out to three significant figures). The necessary level of precision can vary depending on, e.g., the requirements of a given regulatory agency, or the manufacturing process.

In some embodiments, the pharmaceutical composition comprises 1% (w/v) to 4% (w/V) sucrose. In some embodiments, the pharmaceutical composition comprises 2% (w/v) to 5% (w/v) sucrose. In some embodiments, the pharmaceutical composition comprises 4% (w/v) to 8% (w/v) sucrose. In some embodiments, the pharmaceutical composition comprises about 1.5% (w/V) to about 2.5% (w/V) sucrose. In some embodiments, the pharmaceutical composition comprises about 5% (w/v) to about 7.5% (w/v) sucrose.

In some embodiments, the pharmaceutical composition comprises about 1.0% (w/V) sucrose. In some embodiments, the pharmaceutical composition comprises about 1.1% (w/v) sucrose. In some embodiments, the pharmaceutical composition comprises about 1.2% (w/v) sucrose. In some embodiments, the pharmaceutical composition comprises about 1.3% (w/v) sucrose. In some embodiments, the pharmaceutical composition comprises about 1.4% (w/v) sucrose. In some embodiments, the pharmaceutical composition comprises about 1.5% (w/v) sucrose. In some embodiments, the pharmaceutical composition comprises about 1.6% (w/v) sucrose. In some embodiments, the pharmaceutical composition comprises about 1.7% (w/v) sucrose. In some embodiments, the pharmaceutical composition comprises about 1.8% (w/v) sucrose. In some embodiments, the pharmaceutical composition comprises about 1.9% (w/V) sucrose. In some embodiments, the pharmaceutical composition comprises about 2.0% (w/v) sucrose. In some embodiments, the pharmaceutical composition comprises about 2.1% (w/v) sucrose. In some embodiments, the pharmaceutical composition comprises about 2.2% (w/v) sucrose. In some embodiments, the pharmaceutical composition comprises about 2.3% (w/v) sucrose. In some embodiments, the pharmaceutical composition comprises about 2.4% (w/V) sucrose. In some embodiments, the pharmaceutical composition comprises about 2.5% (w/v) sucrose. In some embodiments, the pharmaceutical composition comprises about 2.6% (w/v) sucrose. In some embodiments, the pharmaceutical composition comprises about 2.7% (w/v) sucrose. In some embodiments, the pharmaceutical composition comprises about 2.8% (w/V) sucrose. In some embodiments, the pharmaceutical composition comprises about 2.9% (w/v) sucrose. In some embodiments, the pharmaceutical composition comprises about 3.0% (w/v) sucrose. In some embodiments, the pharmaceutical composition comprises about 3.1% (w/v) sucrose. In some embodiments, the pharmaceutical composition comprises about 3.2% (w/v) sucrose. In some embodiments, the pharmaceutical composition comprises about 3.3% (w/v) sucrose. In some embodiments, the pharmaceutical composition comprises about 3.4% (w/v) sucrose. In some embodiments, the pharmaceutical composition comprises about 3.5% (w/v) sucrose. In some embodiments, the pharmaceutical composition comprises about 3.6% (w/v) sucrose. In some embodiments, the pharmaceutical composition comprises about 3.7% (w/V) sucrose. In some embodiments, the pharmaceutical composition comprises about 3.8% (w/v) sucrose. In some embodiments, the pharmaceutical composition comprises about 3.9% (w/v) sucrose. In some embodiments, the pharmaceutical composition comprises about 4.0% (w/v) sucrose. In some embodiments, the pharmaceutical composition comprises about 4.1% (w/v) sucrose. In some embodiments, the pharmaceutical composition comprises about 4.2% (w/v) sucrose. In some embodiments, the pharmaceutical composition comprises about 4.3% (w/v) sucrose. In some embodiments, the pharmaceutical composition comprises about 4.4% (w/v) sucrose. In some embodiments, the pharmaceutical composition comprises about 4.5% (w/v) sucrose. In some embodiments, the pharmaceutical composition comprises about 4.4% (w/V) sucrose. In some embodiments, the pharmaceutical composition comprises about 4.7% (w/v) sucrose. In some embodiments, the pharmaceutical composition comprises about 4.8% (w/v) sucrose. In some embodiments, the pharmaceutical composition comprises about 4.9% (w/v) sucrose. In some embodiments, the pharmaceutical composition comprises about 5% (w/v) sucrose. In some embodiments, the pharmaceutical composition comprises about 5.1% (w/v) sucrose. In some embodiments, the pharmaceutical composition comprises about 5.2% (w/v) sucrose. In some embodiments, the pharmaceutical composition comprises about 5.3% (w/v) sucrose. In some embodiments, the pharmaceutical composition comprises about 5.4% (w/v) sucrose. In some embodiments, the pharmaceutical composition comprises about 5.5% (w/V) sucrose. In some embodiments, the pharmaceutical composition comprises about 5.6% (w/v) sucrose. In some embodiments, the pharmaceutical composition comprises about 5.7% (w/v) sucrose. In some embodiments, the pharmaceutical composition comprises about 5.8% (w/v) sucrose. In some embodiments, the pharmaceutical composition comprises about 5.9% (w/v) sucrose. In some embodiments, the pharmaceutical composition comprises about 6% (w/v) sucrose. In some embodiments, the pharmaceutical composition comprises about 6.1% (w/v) sucrose. In some embodiments, the pharmaceutical composition comprises about 6.2% (w/V) sucrose. In some embodiments, the pharmaceutical composition comprises about 6.3% (w/v) sucrose. In some embodiments, the pharmaceutical composition comprises about 6.4% (w/V) sucrose. In some embodiments, the pharmaceutical composition comprises about 6.5% (w/v) sucrose. In some embodiments, the pharmaceutical composition comprises about 6.6% (w/v) sucrose. In some embodiments, the pharmaceutical composition comprises about 6.7% (w/v) sucrose. In some embodiments, the pharmaceutical composition comprises about 6.8% (w/v) sucrose. In some embodiments, the pharmaceutical composition comprises about 6.9% (w/V) sucrose. In some embodiments, the pharmaceutical composition comprises about 7% (w/v) sucrose. In some embodiments, the pharmaceutical composition comprises about 7.1% (w/v) sucrose. In some embodiments, the pharmaceutical composition comprises about 7.2% (w/v) sucrose. In some embodiments, the pharmaceutical composition comprises about 7.3% (w/V) sucrose. In some embodiments, the pharmaceutical composition comprises about 7.4% (w/v) sucrose. In some embodiments, the pharmaceutical composition comprises about 7.5% (w/v) sucrose. In some embodiments, the pharmaceutical composition comprises about 7.6% (w/v) sucrose. In some embodiments, the pharmaceutical composition comprises about 7.7% (w/v) sucrose. In some embodiments, the pharmaceutical composition comprises about 7.8% (w/v) sucrose. In some embodiments, the pharmaceutical composition comprises about 7.9% (w/v) sucrose. In some embodiments, the pharmaceutical composition comprises about 8% (w/v) sucrose.

In some embodiments, the pharmaceutical composition comprises about 33.67 mg sucrose. In some embodiments, the pharmaceutical composition comprises about 67.34 mg sucrose. In some embodiments, the pharmaceutical composition comprises about 168.35 mg sucrose. In some embodiments, the amount of sucrose can vary up to 10% of a specific amount. In some embodiments, the amount of sucrose can vary up to 5% of a specific amount. In some embodiments, the amount of sucrose can vary up to 1% of a specific amount. In some embodiments, the specific amount of sucrose is 33.67 mg, 67.34 mg, or 168.35 mg.

Pharmaceutical compositions disclosed herein may include a buffer. In some embodiments, the pharmaceutical compositions disclosed herein include specified amounts or concentrations of histidine. In some embodiments, the histidine included in the pharmaceutical composition is L-histidine. In some embodiments, the pharmaceutical composition comprises about 5 mM to about 15 mM histidine.

In some embodiments, the pharmaceutical composition comprises about 5 mM histidine. In some embodiments, the pharmaceutical composition comprises about 5.5 mM histidine. In some embodiments, the pharmaceutical composition comprises about 6 mM histidine. In some embodiments, the pharmaceutical composition comprises about 6.5 mM histidine. In some embodiments, the pharmaceutical composition comprises about 7 mM histidine. In some embodiments, the pharmaceutical composition comprises about 7.5 mM histidine. In some embodiments, the pharmaceutical composition comprises about 8 mM histidine. In some embodiments, the pharmaceutical composition comprises about 8.5 mM histidine. In some embodiments, the pharmaceutical composition comprises about 9 mM histidine. In some embodiments, the pharmaceutical composition comprises about 9.5 mM histidine. In some embodiments, the pharmaceutical composition comprises about 10 mM histidine. In some embodiments, the pharmaceutical composition comprises about 10.5 mM histidine. In some embodiments, the pharmaceutical composition comprises about 11 mM histidine. In some embodiments, the pharmaceutical composition comprises about 11.5 mM histidine. In some embodiments, the pharmaceutical composition comprises about 12 mM histidine. In some embodiments, the pharmaceutical composition comprises about 12.5 mM histidine. In some embodiments, the pharmaceutical composition comprises about 13 mM histidine. In some embodiments, the pharmaceutical composition comprises about 13.5 mM histidine. In some embodiments, the pharmaceutical composition comprises about 14 mM histidine. In some embodiments, the pharmaceutical composition comprises about 14.5 mM histidine. In some embodiments, the pharmaceutical composition comprises about 15 mM histidine. In some embodiments, the histidine is L-histidine.

In some embodiments, the pharmaceutical compositions disclosed herein include specified amounts or concentrations of arginine. In some embodiments, the the pharmaceutical composition comprises arginine hydrochloride (HCl). In some embodiments, the arginine is L-arginine. In some embodiments, the composition comprises L-arginine-HCl.

In some embodiments, the pharmaceutical composition comprises about 200 mM to about 300 mM arginine.

In some embodiments, the pharmaceutical composition comprises about 200 mM arginine. In some embodiments, the pharmaceutical composition comprises about 210 mM arginine. In some embodiments, the pharmaceutical composition comprises about 220 mM arginine.

In some embodiments, the pharmaceutical composition comprises about 230 mM arginine. In some embodiments, the pharmaceutical composition comprises about 240 mM arginine. In some embodiments, the pharmaceutical composition comprises about 250 mM arginine. In some embodiments, the pharmaceutical composition comprises about 260 mM arginine. In some embodiments, the pharmaceutical composition comprises about 270 mM arginine. In some embodiments, the pharmaceutical composition comprises about 280 mM arginine. In some embodiments, the pharmaceutical composition comprises about 290 mM arginine. In some embodiments, the pharmaceutical composition comprises about 300 mM arginine. In some embodiments, the arginine is L-arginine. In some embodiments, the composition comprises L-arginine-HCl.

Pharmaceutical compositions disclosed herein may include a bulking agent. In some embodiments, the pharmaceutical compositions disclosed herein include specified amounts or concentrations of calcium chloride (CaCl₂)). In some embodiments, the composition comprises CaCl₂)·2H₂O, CaCl₂) (anhydrous), CaCl₂)·4H₂O, or CaCl₂)·6H₂O. In some embodiments, the composition comprises calcium chloride dihydrate. In some embodiments, the pharmaceutical composition comprises about 2.5 mM to about 10 mM calcium chloride. In some embodiments, the composition comprises calcium chloride dihydrate.

In some embodiments, the pharmaceutical composition comprises about 2.5 mM calcium chloride. In some embodiments, the pharmaceutical composition comprises about 3 mM calcium chloride. In some embodiments, the pharmaceutical composition comprises about 3.5 mM calcium chloride. In some embodiments, the pharmaceutical composition comprises about 4 mM calcium chloride. In some embodiments, the pharmaceutical composition comprises about 4.5 mM calcium chloride. In some embodiments, the pharmaceutical composition comprises about 5 mM calcium chloride. In some embodiments, the pharmaceutical composition comprises about 5.5 mM calcium chloride. In some embodiments, the pharmaceutical composition comprises about 6 mM calcium chloride. In some embodiments, the pharmaceutical composition comprises about 6.5 mM calcium chloride. In some embodiments, the pharmaceutical composition comprises about 7 mM calcium chloride. In some embodiments, the pharmaceutical composition comprises about 7.5 mM calcium chloride. In some embodiments, the pharmaceutical composition comprises about 8 mM calcium chloride. In some embodiments, the pharmaceutical composition comprises about 8.5 mM calcium chloride. In some embodiments, the pharmaceutical composition comprises about 9 mM calcium chloride. In some embodiments, the pharmaceutical composition comprises about 9.5 mM calcium chloride. In some embodiments, the pharmaceutical composition comprises about 10 mM calcium chloride. In some embodiments, the composition comprises calcium chloride dihydrate.

In some embodiments, the pharmaceutical compositions disclosed herein do not include bulking agents other than calcium chloride. In some embodiments, calcium chloride is the sole bulking agent. In some embodiments, the pharmaceutical composition comprises less than 8.8 mg/mL sodium chloride (NaCl). In some embodiments, the pharmaceutical composition is substantially free of sodium chloride. In some embodiments, the pharmaceutical composition is free of sodium chloride.

In some embodiments, the pharmaceutical compositions disclosed herein include specified amounts or concentrations of poloxamer 188 (P188). In some embodiments, the poloxamer is poloxamer 101, 105, 108, 122, 123, 124, 181, 182, 183, 184, 185, 188, 212, 215, 217, 231, 234, 235, 237, 238, 282, 284, 288, 331, 333, 334, 335, 338, 401, 402, 403, or 407. In some embodiments, the poloxamer is poloxamer 407. In some embodiments, the pharmaceutical composition comprises about 0.01 mg/ml to about 10 mg/ml of a poloxamer. In some embodiments, the pharmaceutical composition comprises at least about 1 mg/ml of a poloxamer.

In some embodiments, the pharmaceutical compositions disclosed herein include specified amounts or concentrations of poloxamer 188 (P188). Non-limiting disclosures relating to P188 may be found in Strickley and Lambert (2021) Journal of Pharmaceutical Sciences 110 2590-2608, the entire contents of each of which are incorporated herein by reference. In some embodiments, the pharmaceutical composition comprises about 0.01% (w/v) to about 1.0% (w/v) poloxamer 188. In some embodiments, the pharmaceutical composition comprises at least about 0.1% poloxamer 188. In some embodiments, the pharmaceutical composition comprises about 0.1% poloxamer 188.

In some embodiments, the pharmaceutical composition comprises about 0.01% (w/V) poloxamer 188. In some embodiments, the pharmaceutical composition comprises about 0.02% (w/v) poloxamer 188. In some embodiments, the pharmaceutical composition comprises about 0.03% (w/v) poloxamer 188. In some embodiments, the pharmaceutical composition comprises about 0.04% (w/v) poloxamer 188. In some embodiments, the pharmaceutical composition comprises about 0.05% (w/V) poloxamer 188. In some embodiments, the pharmaceutical composition comprises about 0.06% (w/v) poloxamer 188. In some embodiments, the pharmaceutical composition comprises about 0.07% (w/v) poloxamer 188. In some embodiments, the pharmaceutical composition comprises about 0.08% (w/v) poloxamer 188. In some embodiments, the pharmaceutical composition comprises about 0.09% (w/v) poloxamer 188. In some embodiments, the pharmaceutical composition comprises about 0.1% (w/v) poloxamer 188.

In some embodiments, the pharmaceutical composition comprises about 0.11% (w/V) poloxamer 188. In some embodiments, the pharmaceutical composition comprises about 0.12% (w/v) poloxamer 188. In some embodiments, the pharmaceutical composition comprises about 0.13% (w/v) poloxamer 188. In some embodiments, the pharmaceutical composition comprises about 0.14% (w/v) poloxamer 188. In some embodiments, the pharmaceutical composition comprises about 0.15% (w/v) poloxamer 188. In some embodiments, the pharmaceutical composition comprises about 0.16% (w/v) poloxamer 188. In some embodiments, the pharmaceutical composition comprises about 0.17% (w/v) poloxamer 188. In some embodiments, the pharmaceutical composition comprises about 0.18% (w/v) poloxamer 188. In some embodiments, the pharmaceutical composition comprises about 0.19% (w/v) poloxamer 188. In some embodiments, the pharmaceutical composition comprises about 0.2% (w/v) poloxamer 188.

In some embodiments, the pharmaceutical composition comprises about 0.3% (w/v) poloxamer 188. In some embodiments, the pharmaceutical composition comprises about 0.4% (w/v) poloxamer 188. In some embodiments, the pharmaceutical composition comprises about 0.5% (w/v) poloxamer 188. In some embodiments, the pharmaceutical composition comprises about 0.6% (w/v) poloxamer 188. In some embodiments, the pharmaceutical composition comprises about 0.7% (w/v) poloxamer 188. In some embodiments, the pharmaceutical composition comprises about 0.8% (w/v) poloxamer 188. In some embodiments, the pharmaceutical composition comprises about 0.9% (w/v) poloxamer 188. In some embodiments, the pharmaceutical composition comprises about 1.0% (w/v) poloxamer 188.

In some embodiments, the pharmaceutical composition is a pre-lyophilization solution. In some embodiments, pre-lyophilization solution does not comprise NaCl.

In some embodiments, the pharmaceutical composition comprises
(a) about 1% (w/v) to about 8% (w/v) sucrose;
(b) about 5 mM to about 15 mM histidine;
(c) about 200 mM to about 300 mM arginine;
(d) about 2.5 mM to about 10 mM calcium chloride; and
(e) about 0.1% (w/v) to about 1.0% (w/v) poloxamer. In some embodiments, the poloxamer is poloxamer 188 (P188). In some embodiments, the composition comprises poloxamer 188. In some embodiments, the histidine is L-histidine. In some embodiments, the arginine is L-arginine. In some embodiments the composition comprises L-arginine-HCl. In some embodiments, the composition comprises calcium chloride dihydrate. In some embodiments, the pharmaceutical composition is a pre-lyophilization solution.

In some embodiments, when the lyophilized pharmaceutical composition and the sterile water are combined, then the resulting solution does not comprise NaCl.

In some embodiments, the pharmaceutical composition comprises
(a) about 1% (w/v) sucrose;
(b) about 10 mM histidine;
(c) about 250 mM arginine;
(d) about 5 mM calcium chloride; and
(e) about 0.1% poloxamer 188. In some embodiments, the histidine is L-histidine. In some embodiments, the arginine is L-arginine. In some embodiments the composition comprises L-arginine-HCl. In some embodiments, the composition comprises calcium chloride dihydrate. In some embodiments, the pharmaceutical composition is a pre-lyophilization solution.

In some embodiments, the pharmaceutical composition comprises
(a) about 2% (w/v) sucrose;
(b) about 10 mM histidine;
(c) about 250 mM arginine;
(d) about 5 mM calcium chloride; and
(e) about 0.1% poloxamer 188. In some embodiments, the histidine is L-histidine. In some embodiments, the arginine is L-arginine. In some embodiments the composition comprises L-arginine-HCl. In some embodiments, the composition comprises calcium chloride dihydrate. In some embodiments, the pharmaceutical composition is a pre-lyophilization solution.

In some embodiments, the pharmaceutical composition comprises
(a) about 5% (w/v) sucrose;
(b) about 10 mM histidine;
(c) about 250 mM arginine;
(d) about 5 mM calcium chloride; and
(e) about 0.1% poloxamer 188. In some embodiments, the histidine is L-histidine. In some embodiments, the arginine is L-arginine. In some embodiments the composition comprises L-arginine-HCl. In some embodiments, the composition comprises calcium chloride dihydrate. In some embodiments, the pharmaceutical composition is a pre-lyophilization solution. In some embodiments, the pharmaceutical composition comprises about 1% (w/v) to about 5% (w/v) sucrose; (a)
(b) about 10 mM histidine;
(c) about 250 mM arginine;
(d) about 5 mM calcium chloride; and
(e) about 0.1% poloxamer 188. In some embodiments, the histidine is L-histidine. In some embodiments, the arginine is L-arginine. In some embodiments the composition comprises L-arginine-HCl. In some embodiments, the composition comprises calcium chloride dihydrate. In some embodiments, the pharmaceutical composition is a pre-lyophilization solution. In some embodiments, the pharmaceutical composition comprises
(a) about 1%, 2%, or 5% (w/v) sucrose;
(b) about 5 mM to about 15 mM histidine;
(c) about 250 mM arginine;
(d) about 5 mM calcium chloride; and
(e) about 0.1% poloxamer 188. In some embodiments, the histidine is L-histidine. In some embodiments, the arginine is L-arginine. In some embodiments the composition comprises L-arginine-HCl. In some embodiments, the composition comprises calcium chloride dihydrate. In some embodiments, the pharmaceutical composition is a pre-lyophilization solution. In some embodiments, the pharmaceutical composition comprises
(a) about 1%, 2%, or 5% (w/v) sucrose;
(b) about 10 mM histidine;
(c) about 200 mM to about 300 mM arginine;
(d) about 5 mM calcium chloride; and
(e) about 0.1% poloxamer 188. In some embodiments, the histidine is L-histidine. In some embodiments, the arginine is L-arginine. In some embodiments the composition comprises L-arginine-HCl. In some embodiments, the composition comprises calcium chloride dihydrate. In some embodiments, the pharmaceutical composition is a pre-lyophilization solution.

In some embodiments, the pharmaceutical composition comprises
(a) about 1%, 2%, or 5% (w/v) sucrose;
(b) about 10 mM histidine;
(c) about 250 mM arginine;
(d) about 2.5 mM to about 10 mM calcium chloride; and
(e) about 0.1% poloxamer 188. In some embodiments, the histidine is L-histidine. In some embodiments, the arginine is L-arginine. In some embodiments the composition comprises L-arginine-HCl. In some embodiments, the composition comprises calcium chloride dihydrate. In some embodiments, the pharmaceutical composition is a pre-lyophilization solution.

In some embodiments, the pharmaceutical composition comprises
(a) about 1%, 2%, or 5% (w/v) sucrose;
(b) about 10 mM histidine;
(c) about 250 mM arginine;
(d) about 5 mM calcium chloride; and
(e) about 0.008% (w/v) to about 0.1% (w/v) poloxamer 188. In some embodiments, the histidine is L-histidine. In some embodiments, the arginine is L-arginine. In some embodiments the composition comprises L-arginine-HCl. In some embodiments, the composition comprises calcium chloride dihydrate. In some embodiments, the pharmaceutical composition is a pre-lyophilization solution.

In some embodiments, the pharmaceutical composition comprises:
(a) about 10, 20, or 50 mg/ml sucrose;
(b) about 1.552 mg/ml L-histidine;
(c) about 52.665 mg/ml L-arginine-HCl;
(d) about 0.735 mg/ml calcium chloride; and
(e) about 1 mg/ml poloxamer 188.

In some embodiments, the pharmaceutical composition comprises:
(a) about 10, 20, or 50 mg/ml sucrose;
(b) about 1.552 mg/ml L-histidine;
(c) about 52.665 mg/ml L-arginine-HCl;
(d) about 0.735 mg/ml calcium chloride dihydrate; and
(e) about 1 mg/ml poloxamer 188.

In some embodiments, the pharmaceutical composition comprises:
(a) about 10, 20, or 50 mg/ml sucrose;
(b) about 1.552 mg/ml L-histidine;
(c) about 52.665 mg/ml L-arginine-HCl;
(d) about 0.555 mg/ml calcium chloride; and
(e) about 1 mg/ml poloxamer 188. In some embodiments, the composition comprises calcium chloride dihydrate. In some embodiments, the pharmaceutical composition is a pre-lyophilization solution.

In some embodiments, the pharmaceutical composition comprises:
(a) about 10, 20, or 50 mg/ml sucrose;
(b) about 1.552 mg/ml L-histidine;
(c) about 43.550 mg/ml L-arginine;
(d) about 0.735 mg/ml calcium chloride dihydrate; and
(e) about 1 mg/ml poloxamer 188. In some embodiments the composition comprises L-arginine-HCl. In some embodiments, the composition comprises calcium chloride dihydrate. In some embodiments, the pharmaceutical composition is a pre-lyophilization solution.

In some embodiments, the pharmaceutical composition comprises:
(a) about 10, 20, or 50 mg/ml sucrose;
(b) about 1.552 mg/ml L-histidine;
(c) about 43.550 mg/ml . . . arginine;
(d) about 0.735 mg/ml calcium chloride; and
(e) about 1 mg/ml poloxamer 188. In some embodiments the composition comprises L-arginine-HCl. In some embodiments, the composition comprises calcium chloride dihydrate. In some embodiments, the pharmaceutical composition is a pre-lyophilization solution.

In some embodiments, the pharmaceutical composition comprises:
(a) about 10, 20, or 50 mg/ml sucrose;
(b) about 1.552 mg/ml L-histidine;
(c) about 43.550 mg/ml L-arginine;
(d) about 0.555 mg/ml calcium chloride; and
(e) about 1 mg/ml poloxamer 188. In some embodiments the composition comprises L-arginine-HCl. In some embodiments, the composition comprises calcium chloride dihydrate. In some embodiments, the pharmaceutical composition is a pre-lyophilization solution.

In some embodiments, the pharmaceutical composition comprises:
(a) 11.23, 22.45, or 56.12 mg/ml sucrose;
(b) 1.74 mg/ml L-histidine;
(c) 59.11 mg/ml L-arginine-HCl;
(d) 0.82 mg/ml calcium chloride dihydrate; and
(e) 1.12 mg/ml poloxamer 188.

In some embodiments, the pharmaceutical composition comprises:
(a) 11.23, 22.45, or 56.12 mg/ml sucrose;
(b) 1.74 mg/ml L-histidine;
(c) 59.11 mg/ml L-arginine-HCl;
(d) 0.62 mg/ml calcium chloride; and
(e) 1.12 mg/ml poloxamer 188. In some embodiments, the composition comprises calcium chloride dihydrate.

In some embodiments, the pharmaceutical composition comprises:
(a) 11.23, 22.45, or 56.12 mg/ml sucrose;
(b) 1.74 mg/ml L-histidine;
(c) 48.88 mg/ml L-arginine;
(d) 0.82 mg/ml calcium chloride dihydrate; and
(e) 1.12 mg/ml poloxamer 188.

In some embodiments, the pharmaceutical composition comprises:
(a) 11.23, 22.45, or 56.12 mg/ml sucrose;
(b) 1.74 mg/ml L-histidine;
(c) 48.88 mg/ml L-arginine;
(d) 0.62 mg/ml calcium chloride; and
(e) 1.12 mg/ml poloxamer 188. In some embodiments the composition comprises L-arginine-HCl. In some embodiments, the composition comprises calcium chloride dihydrate.

In some embodiments, the pharmaceutical composition has a pH of about 6.5 to about 7.5. In some embodiments, the pharmaceutical composition has a pH of about 7.0. In some embodiments, the pharmaceutical composition has a pH of about 6.8.

In some embodiments, the pharmaceutical composition has a pH of about 6.5. In some embodiments, the pharmaceutical composition has a pH of about 6.6. In some embodiments, the pharmaceutical composition has a pH of about 6.7. In some embodiments, the pharmaceutical composition has a pH of about 6.8. In some embodiments, the pharmaceutical composition has a pH of about 6.9. In some embodiments, the pharmaceutical composition has a pH of about 7.0. In some embodiments, the pharmaceutical composition has a pH of about 7.1. In some embodiments, the pharmaceutical composition has a pH of about 7.2. In some embodiments, the pharmaceutical composition has a pH of about 7.3. In some embodiments, the pharmaceutical composition has a pH of about 7.4. In some embodiments, the pharmaceutical composition has a pH of about 7.5.

In some embodiments, the pharmaceutical composition has a pH of 6.5. In some embodiments, the pharmaceutical composition has a pH of 6.6. In some embodiments, the pharmaceutical composition has a pH of 6.7. In some embodiments, the pharmaceutical composition has a pH of 6.8. In some embodiments, the pharmaceutical composition has a pH of 6.9. In some embodiments, the pharmaceutical composition has a pH of 7.0. In some embodiments, the pharmaceutical composition has a pH of 7.1. In some embodiments, the pharmaceutical composition has a pH of 7.2. In some embodiments, the pharmaceutical composition has a pH of 7.3. In some embodiments, the pharmaceutical composition has a pH of 7.4. In some embodiments, the pharmaceutical composition has a pH of 7.5.

In some embodiments, a volume of 3.367 mL of the pre-lyophilization solution is added to a container or vial. In some embodiments, the pre-lyophilized solution is subjected to lyophilization, resulting in a lyophilized pharmaceutical composition.

In some embodiments, the lyophilized pharmaceutical composition comprises:
(a) about 10 mg to about 200 mg sucrose;
(b) about 2.5 mg to about 7.5 mg histidine;
(c) about 140 mg to about 200 mg arginine;
(d) about 1.5 mg to about 5 mg calcium chloride; and
(e) about 1 mg to about 10 mg poloxamer 188.

In some embodiments, the lyophilized pharmaceutical composition comprises:
(a) about 10 mg to about 200 mg sucrose;
(b) about 2.5 mg to about 7.5 mg L-histidine;
(c) about 140 mg to about 200 mg L-arginine;
(d) about 1.5 mg to about 5 mg calcium chloride; and
(e) about 1 mg to about 10 mg poloxamer 188. In some embodiments the composition comprises L-arginine-HCl. In some embodiments, the composition comprises calcium chloride dihydrate.

In some embodiments, the lyophilized pharmaceutical composition comprises:
(a) 10 mg to 200 mg sucrose;
(b) 2.5 mg to 7.5 mg histidine;
(c) 140 mg to 200 mg arginine;
(d) 1.5 mg to 5 mg calcium chloride; and
(e) 1 mg to 10 mg poloxamer 188.

In some embodiments, the lyophilized pharmaceutical composition comprises:
(a) 10 mg to 200 mg sucrose;
(b) 2.5 mg to 7.5 mg L-histidine;
(c) 140 mg to 200 mg L-arginine-HCl;
(d) 1.5 mg to 5 mg calcium chloride; and
(e) 1 mg to 10 mg poloxamer 188. In some embodiments, the composition comprises calcium chloride dihydrate.

In some embodiments, the lyophilized pharmaceutical composition comprises:
(a) 10 mg to 200 mg sucrose;
(b) 2.5 mg to 7.5 mg L-histidine;
(c) 140 mg to 200 mg L-arginine;
(d) 1.5 mg to 5 mg calcium chloride; and
(e) 1 mg to 10 mg poloxamer 188. In some embodiments the composition comprises L-arginine-HCl. In some embodiments, the composition comprises calcium chloride dihydrate.

In some embodiments, the lyophilized pharmaceutical composition comprises:
(a) about 33.7, 67.3, or 168.4 mg sucrose;
(b) about 5.2 mg L-histidine;
(c) about 177.3 mg L-arginine-HCl;
(d) about 2.5 mg calcium chloride; and
(e) about 3.4 mg poloxamer 188.

In some embodiments, the lyophilized pharmaceutical composition comprises:
(a) about 33.7, 67.3, or 168.4 mg sucrose;
(b) about 5.2 mg L-histidine;
(c) about 146.6 mg L-arginine;
(d) about 2.5 mg calcium chloride; and
(e) about 3.4 mg poloxamer 188.

In some embodiments, the lyophilized pharmaceutical composition comprises:
(a) 33.7, 67.3, or 168.4 mg sucrose;
(b) 5.2 mg L-histidine;
(c) 177.3 mg L-arginine-HCl;
(d) 2.5 mg calcium chloride; and
(e) 3.4 mg poloxamer 188.

In some embodiments, the lyophilized pharmaceutical composition comprises:
(a) 33.7, 67.3, or 168.4 mg sucrose;
(b) 5.2 mg L-histidine;
(c) 146.6 mg L-arginine;
(d) 2.5 mg calcium chloride; and
(e) 3.4 mg poloxamer 188.

In some embodiments, the lyophilized pharmaceutical composition comprises:
(a) 33.67, 67.34, or 168.35 mg sucrose;
(b) 5.23 mg L-histidine;
(c) 177.32 mg L-arginine-HCl;
(d) 2.47 mg calcium chloride dihydrate; and
(e) 3.37 mg poloxamer 188. In some embodiments, the lyophilized pharmaceutical composition comprises:
(a) 33.67, 67.34, or 168.35 mg sucrose;
(b) 5.23 mg L-histidine;
(c) 177.32 mg L-arginine-HCl;
(d) 1.87 mg calcium chloride; and
(e) 3.37 mg poloxamer 188.

In some embodiments, the lyophilized pharmaceutical composition comprises:
(a) 33.67, 67.34, or 168.35 mg sucrose;
(b) 5.23 mg L-histidine;
(c) 146.63 mg L-arginine;
(d) 2.47 mg calcium chloride dihydrate; and
(e) 3.37 mg poloxamer 188.

In some embodiments, the lyophilized pharmaceutical composition comprises:
(a) 33.67, 67.34, or 168.35 mg sucrose;
(b) 5.23 mg L-histidine;
(c) 146.63 mg L-arginine;
(d) 1.87 mg calcium chloride; and
(e) 3.37 mg poloxamer 188.

In some embodiments, the lyophilized pharmaceutical composition comprises:
(a) 10 mg to 200 mg sucrose;
(b) 5.2 mg L-histidine;
(c) 177.3 mg L-arginine-HCl;
(d) 2.5 mg calcium chloride dihydrate; and
(e) 3.4 mg poloxamer 188.

In some embodiments, the lyophilized pharmaceutical composition comprises:
(a) 10 mg to 200 mg sucrose;
(b) 5.2 mg L-histidine;
(c) 177.3 mg L-arginine-HCl;
(d) 1.9 mg calcium chloride; and
(e) 3.4 mg poloxamer 188.

In some embodiments, the lyophilized pharmaceutical composition comprises:
(a) 10 mg to 200 mg sucrose;
(b) 5.2 mg L-histidine;
(c) 146.6 mg L-arginine;
(d) 2.5 mg calcium chloride dihydrate; and
(e) 3.4 mg poloxamer 188.

In some embodiments, the lyophilized pharmaceutical composition comprises:
(a) 10 mg to 200 mg sucrose;
(b) 5.2 mg L-histidine;
(c) 146.6 mg L-arginine;
(d) 1.9 mg calcium chloride; and
(e) 3.4 mg poloxamer 188.

In some embodiments, the lyophilized pharmaceutical composition comprises:
(a) 33.7, 67.3, or 168.4 mg sucrose;
(b) 2.5 mg to 7.5 mg L-histidine;
(c) 177.3 mg L-arginine-HCl;
(d) 2.5 mg calcium chloride dihydrate; and
(e) 3.4 mg poloxamer 188.

In some embodiments, the lyophilized pharmaceutical composition comprises:
(a) 33.7, 67.3, or 168.4 mg sucrose;
(b) 2.5 mg to 7.5 mg L-histidine;
(c) 177.3 mg L-arginine-HCl;
(d) 1.9 mg calcium chloride; and
(e) 3.4 mg poloxamer 188.

In some embodiments, the lyophilized pharmaceutical composition comprises:
(a) 33.7, 67.3, or 168.4 mg sucrose;
(b) 2.5 mg to 7.5 mg L-histidine;
(c) 146.6 mg L-arginine;
(d) 2.5 mg calcium chloride dihydrate; and
(e) 3.4 mg poloxamer 188.

In some embodiments, the lyophilized pharmaceutical composition comprises:
(a) 33.7, 67.3, or 168.4 mg sucrose;
(b) 2.5 mg to 7.5 mg L-histidine;
(c) 146.6 mg L-arginine;
(d) 1.9 mg calcium chloride; and
(e) 3.4 mg poloxamer 188.

In some embodiments, the lyophilized pharmaceutical composition comprises:
(a) 33.67, 67.34, or 168.35 mg sucrose;
(b) 5.23 mg L-histidine;
(c) 140 mg to 200 mg L-arginine;
(d) 1.5 mg to 5 mg calcium chloride; and
(e) 3.37 mg poloxamer 188. In some embodiments, the composition compriuses 140 mg to 200 mg L-arginine-HCl.

In some embodiments, the lyophilized pharmaceutical composition comprises:
(a) 33.67, 67.34, or 168.35 mg sucrose;
(b) 5.23 mg L-histidine;
(c) 177.32 mg L-arginine-HCl;
(d) 1.5 mg to 5 mg calcium chloride; and
(e) 3.37 mg poloxamer 188.

In some embodiments, the lyophilized pharmaceutical composition comprises:
(a) 33.67, 67.34, or 168.35 mg sucrose;
(b) 5.23 mg L-histidine;
(c) 146.63 mg L-arginine;
(d) 1.5 mg to 5 mg calcium chloride; and
(e) 3.37 mg poloxamer 188.

In some embodiments, the lyophilized pharmaceutical composition comprises:
(a) 33.67, 67.34, or 168.35 mg sucrose;
(b) 5.23 mg L-histidine;
(c) 177.32 mg L-arginine-HCl;
(d) 2.47 mg calcium chloride dihydrate; and
(e) 1 mg to 10 mg poloxamer 188.

In some embodiments, the lyophilized pharmaceutical composition comprises:
(a) 33.67, 67.34, or 168.35 mg sucrose;
(b) 5.23 mg L-histidine;
(c) 177.32 mg L-arginine-HCl;
(d) 1.87 mg calcium chloride; and
(e) 1 mg to 10 mg poloxamer 188.

In some embodiments, the lyophilized pharmaceutical composition comprises:
(a) 33.67, 67.34, or 168.35 mg sucrose;
(b) 5.23 mg L-histidine;
(c) 146.63 mg L-arginine;
(d) 2.47 mg calcium chloride dihydrate; and
(e) 1 mg to 10 mg poloxamer 188.

In some embodiments, the lyophilized pharmaceutical composition comprises:
(a) 33.67, 67.34, or 168.35 mg sucrose;
(b) 5.23 mg L-histidine;
(c) 146.63 mg L-arginine;
(d) 1.87 mg calcium chloride; and
(e) 1 mg to 10 mg poloxamer 188.

In some embodiments, the lyophilized pharmaceutical composition has a moisture content of less than 2%. In some embodiments, the lyophilized pharmaceutical composition has a moisture content of less than 1.8%. In some embodiments, the lyophilized pharmaceutical composition has a moisture content of less than 1.6%.

In some embodiments, the lyophilized pharmaceutical composition is in a lyophilized cake. In some embodiments, the lyophilized cake is white. In some embodiments, the lyophilized cake is less than Y4 in the European Pharmacopoeia color scale. SeeDegree of Coloration of Liquids (Method 2.2.2), European Pharmacopoeia, 10^{th} Ed. (2021).

In some embodiments, the lyophilized pharmaceutical composition and sterile water are combined to produce an injectable solution. In some embodiments, the lyophilized pharmaceutical composition is combined with about 2 mL to about 5 mL of sterile water. In some embodiments, the lyophilized pharmaceutical composition is combined with about 3 mL of sterile water. In some embodiments, the lyophilized pharmaceutical composition is combined with 3 mL of sterile water. In some embodiments, the sterile water is USP grade sterile water. In some embodiments, the sterile water is USP grade sterile water for injection. In some embodiments, the sterile water is pyrogen-free or nonpyrogenic. In some embodiments, the sterile water does not contain a bacteriostatic or antimicrobial agent. In some embodiments, the sterile water contains a bacteriostatic or antimicrobial agent. In some embodiments, the sterile water is sterilized using a filter. In some embodiments, the sterile water is sterilized using a 0.1 μm filter. In some embodiments, the sterile water is distilled water. In some embodiments, the sterile water is sterile, nonpyrogenic, distilled water, hypotonic, with an osmolarity of zero mOsmol/L, and does not contain a bacteriostatic or antimicrobial agent.

In some embodiments, when the lyophilized pharmaceutical composition and the sterile water are combined, then the resulting solution comprises:
(a) 10 mg/mL to 60 mg/mL sucrose;
(b) 1.5 mg/mL to 2.0 mg/mL L-histidine;
(c) 50 mg/mL to 70 mg/mL L-arginine;
(d) 0.7 mg/mL to 0.9 mg/mL calcium chloride dihydrate; and
(e) 0.6 mg/mL to 1.6 mg/mL poloxamer 188. In some embodiments the composition comprises L-arginine-HCl.

In some embodiments, when the lyophilized pharmaceutical composition and the sterile water are combined, then the resulting solution comprises:
(a) 10 mg/mL to 60 mg/mL sucrose;
(b) 1.5 mg/mL to 2.0 mg/mL L-histidine;
(c) 50 mg/mL to 70 mg/mL L-arginine;
(d) 0.5 mg/mL to 0.8 mg/mL calcium chloride; and
(e) 0.6 mg/mL to 1.6 mg/mL poloxamer 188. In some embodiments the composition comprises L-arginine-HCl. In some embodiments, the composition comprises calcium chloride dihydrate

In some embodiments, when the lyophilized pharmaceutical composition and the sterile water are combined, then the resulting solution comprises:
(a) about 11.23, 22.45, or 56.12 mg/ml sucrose;
(b) about 1.74 mg/ml L-histidine;
(c) about 59.11 mg/ml L-arginine-HCl;
(d) about 0.82 mg/ml calcium chloride dihydrate; and
(e) about 1.12 mg/ml poloxamer 188.

In some embodiments, when the lyophilized pharmaceutical composition and the sterile water are combined, then the resulting solution comprises:
(a) about 11.23, 22.45, or 56.12 mg/ml sucrose;
(b) about 1.74 mg/ml L-histidine;
(c) about 59.11 mg/ml L-arginine-HCl;
(d) about 0.62 mg/ml calcium chloride; and
(e) about 1.12 mg/ml poloxamer 188. In some embodiments, the composition comprises calcium chloride dihydrate.

In some embodiments, when the lyophilized pharmaceutical composition and the sterile water are combined, then the resulting solution comprises:
(a) about 11.23, 22.45, or 56.12 mg/ml sucrose;
(b) about 1.74 mg/ml L-histidine;
(c) about 48.88 mg/ml L-arginine;
(d) about 0.82 mg/ml calcium chloride dihydrate; and
(e) about 1.12 mg/ml poloxamer 188.

In some embodiments, when the lyophilized pharmaceutical composition and the sterile water are combined, then the resulting solution comprises:
(a) about 11.23, 22.45, or 56.12 mg/ml sucrose;
(b) about 1.74 mg/ml L-histidine;
(c) about 48.88 mg/ml L-arginine;
(d) about 0.62 mg/ml calcium chloride; and
(e) about 1.12 mg/ml poloxamer 188. In some embodiments the composition comprises L-arginine-HCl. In some embodiments, the composition comprises calcium chloride dihydrate.

In some embodiments, when the lyophilized pharmaceutical composition and the sterile water are combined, then the resulting solution comprises:
(a) about 10 mg/mL to about 60 mg/ml sucrose;
(b) about 1.74 mg/ml L-histidine;
(c) about 59.11 mg/ml L-arginine-HCl;
(d) about 0.82 mg/ml calcium chloride dihydrate; and
(e) about 1.12 mg/ml poloxamer 188.

In some embodiments, when the lyophilized pharmaceutical composition and the sterile water are combined, then the resulting solution comprises:
(a) about 10 mg/mL to about 60 mg/ml sucrose;
(b) about 1.74 mg/ml L-histidine;
(c) about 59.11 mg/ml L-arginine-HCl;
(d) about 0.62 mg/ml calcium chloride; and
(e) about 1.12 mg/ml poloxamer 188. In some embodiments, the composition comprises calcium chloride dihydrate.

In some embodiments, when the lyophilized pharmaceutical composition and the sterile water are combined, then the resulting solution comprises:
(a) about 10 mg/mL to about 60 mg/ml sucrose;
(b) about 1.74 mg/ml L-histidine;
(c) about 44.88 mg/ml L-arginine;
(d) about 0.82 mg/ml calcium chloride dihydrate; and
(e) about 1.12 mg/ml poloxamer 188

In some embodiments, when the lyophilized pharmaceutical composition and the sterile water are combined, then the resulting solution comprises:
(a) about 10 mg/mL to about 60 mg/ml sucrose;
(b) about 1.74 mg/ml L-histidine;
(c) about 44.88 mg/ml L-arginine;
(d) about 0.62 mg/ml calcium chloride; and
(e) about 1.12 mg/ml poloxamer 188. In some embodiments the composition comprises L-arginine-HCl. In some embodiments, the composition comprises calcium chloride dihydrate.

In some embodiments, when the lyophilized pharmaceutical composition and the sterile water are combined, then the resulting solution comprises:
(a) about 11.23, 22.45, or 56.12 mg/ml sucrose;
(b) about 1.5 mg/mL to about 2.0 mg/mL L-histidine;
(c) about 59.11 mg/ml L-arginine-HCl;
(d) about 0.82 mg/ml calcium chloride dihydrate; and
(e) about 1.12 mg/ml poloxamer 188.

In some embodiments, when the lyophilized pharmaceutical composition and the sterile water are combined, then the resulting solution comprises:
(a) about 11.23, 22.45, or 56.12 mg/ml sucrose;
(b) about 1.5 mg/mL to about 2.0 mg/mL L-histidine;
(c) about 59.11 mg/ml L-arginine-HCl;
(d) about 0.62 mg/ml calcium chloride; and
(e) about 1.12 mg/ml poloxamer 188. In some embodiments, the composition comprises calcium chloride dihydrate.

In some embodiments, when the lyophilized pharmaceutical composition and the sterile water are combined, then the resulting solution comprises:
(a) about 11.23, 22.45, or 56.12 mg/ml sucrose;
(b) about 1.5 mg/mL to about 2.0 mg/mL L-histidine;
(c) about 48.88 mg/ml L-arginine;
(d) about 0.82 mg/ml calcium chloride dihydrate; and
(e) about 1.12 mg/ml poloxamer 188.

In some embodiments, when the lyophilized pharmaceutical composition and the sterile water are combined, then the resulting solution comprises:
(a) about 22.45 mg/ml sucrose;
(b) about 1.5 mg/mL to about 2.0 mg/mL L-histidine;
(c) about 48.88 mg/ml L-arginine;
(d) about 0.62 mg/ml calcium chloride; and
(e) about 1.12 mg/ml poloxamer 188. In some embodiments the composition comprises L-arginine-HCl. In some embodiments, the composition comprises calcium chloride dihydrate.

In some embodiments, when the lyophilized pharmaceutical composition and the sterile water are combined, then the resulting solution comprises:
(a) about 11.23, 22.45, or 56.12 mg/ml sucrose;
(b) about 1.74 mg/ml L-histidine;
(c) about 50 mg/mL to about 70 mg/mL L-arginine-HCl;
(d) about 0.62 mg/ml calcium chloride dihydrate; and
(e) about 1.12 mg/ml poloxamer 188.

In some embodiments, when the lyophilized pharmaceutical composition and the sterile water are combined, then the resulting solution comprises:
(a) about 11.23, 22.45, or 56.12 mg/ml sucrose;
(b) about 1.74 mg/ml L-histidine;
(c) about 50 mg/mL to about 70 mg/mL L-arginine-HCl;
(d) about 0.62 mg/ml calcium chloride; and
(e) about 1.12 mg/ml poloxamer 188. In some embodiments, the composition comprises calcium chloride dihydrate.

In some embodiments, when the lyophilized pharmaceutical composition and the sterile water are combined, then the resulting solution comprises:
(a) about 11.23, 22.45, or 56.12 mg/ml sucrose;
(b) about 1.74 mg/ml L-histidine;
(c) about 40 mg/mL to about 60 mg/mL L-arginine;
(d) about 0.82 mg/ml calcium chloride dihydrate; and
(e) about 1.12 mg/ml poloxamer 188.

In some embodiments, when the lyophilized pharmaceutical composition and the sterile water are combined, then the resulting solution comprises:
(a) about 11.23, 22.45, or 56.12 mg/ml sucrose;
(b) about 1.74 mg/ml L-histidine;
(c) about 40 mg/mL to about 60 mg/mL L-arginine;
(d) about 0.62 mg/ml calcium chloride; and
(e) about 1.12 mg/ml poloxamer 188. In some embodiments the composition comprises L-arginine-HCl. In some embodiments, the composition comprises calcium chloride dihydrate.

In some embodiments, when the lyophilized pharmaceutical composition and the sterile water are combined, then the resulting solution comprises:
(a) about 11.23, 22.45, or 56.12 mg/ml sucrose;
(b) about 1.74 mg/ml L-histidine;
(c) about 59.11 mg/ml L-arginine-HCl;
(d) about 0.7 mg/mL to about 0.9 mg/mL calcium chloride dihydrate; and
(e) about 1.12 mg/ml poloxamer 188.

In some embodiments, when the lyophilized pharmaceutical composition and the sterile water are combined, then the resulting solution comprises:
(a) about 11.23, 22.45, or 56.12 mg/ml sucrose;
(b) about 1.74 mg/ml L-histidine;
(c) about 59.11 mg/ml L-arginine-HCl;
(d) about 0.5 mg/mL to about 0.9 mg/mL calcium chloride; and
(e) about 1.12 mg/ml poloxamer 188. In some embodiments, the composition comprises calcium chloride dihydrate.

In some embodiments, when the lyophilized pharmaceutical composition and the sterile water are combined, then the resulting solution comprises:
(a) about 11.23, 22.45, or 56.12 mg/ml sucrose;
(b) about 1.74 mg/ml L-histidine;
(c) about 48.88 mg/ml L-arginine;
(d) about 0.7 mg/mL to about 0.9 mg/mL calcium chloride dihydrate; and
(e) about 1.12 mg/ml poloxamer 188. In some embodiments the composition comprises L-arginine-HCl.

In some embodiments, when the lyophilized pharmaceutical composition and the sterile water are combined, then the resulting solution comprises:
(a) about 11.23, 22.45, or 56.12 mg/ml sucrose;
(b) about 1.74 mg/ml L-histidine;
(c) about 48.88 mg/ml L-arginine;
(d) about 0.5 mg/mL to about 0.7 mg/mL calcium chloride; and
(e) about 1.12 mg/ml poloxamer 188. In some embodiments the composition comprises L-arginine-HCl. In some embodiments, the composition comprises calcium chloride dihydrate.

In some embodiments, when the lyophilized pharmaceutical composition and the sterile water are combined, then the resulting solution comprises:
(a) about 11.23, 22.45, or 56.12 mg/ml sucrose;
(b) about 1.74 mg/ml L-histidine;
(c) about 59.11 mg/ml L-arginine-HCl;
(d) about 0.82 mg/ml calcium chloride dihydrate; and
(e) about 0.6 mg/mL to about 1.6 mg/mL poloxamer 188.

In some embodiments, when the lyophilized pharmaceutical composition and the sterile water are combined, then the resulting solution comprises:
(a) about 11.23, 22.45, or 56.12 mg/ml sucrose;
(b) about 1.74 mg/ml L-histidine;
(c) about 59.11 mg/ml L-arginine-HCl;
(d) about 0.62 mg/ml calcium chloride; and
(e) about 0.6 mg/mL to about 1.6 mg/mL poloxamer 188. In some embodiments the composition comprises L-arginine-HCl. In some embodiments, the composition comprises calcium chloride dihydrate.

In some embodiments, when the lyophilized pharmaceutical composition and the sterile water are combined, then the resulting solution comprises:
(a) about 11.23, 22.45, or 56.12 mg/ml sucrose;
(b) about 1.74 mg/ml L-histidine;
(c) about 48.88 mg/ml L-arginine;
(d) about 0.82 mg/ml calcium chloride dihydrate; and
(e) about 0.6 mg/mL to about 1.6 mg/mL poloxamer 188.

In some embodiments, when the lyophilized pharmaceutical composition and the sterile water are combined, then the resulting solution comprises:
(a) about 11.23, 22.45, or 56.12 mg/ml sucrose;
(b) about 1.74 mg/ml L-histidine;
(c) about 48.88 mg/ml L-arginine;
(d) about 0.62 mg/ml calcium chloride; and
(e) about 0.6 mg/mL to about 1.6 mg/mL poloxamer 188. In some embodiments the composition comprises L-arginine-HCl. In some embodiments, the composition comprises calcium chloride dihydrate.

In some embodiments, when the lyophilized pharmaceutical composition and the sterile water are combined, then the resulting solution comprises:
(a) about 11.23, 22.45, or 56.12 mg/ml sucrose;
(b) about 1.74 mg/ml L-histidine;
(c) about 59.11 mg/ml L-arginine-HCl;
(d) about 0.82 mg/ml calcium chloride dihydrate; and
(e) about 1.12 mg/ml poloxamer 188.

In some embodiments, when the lyophilized pharmaceutical composition and the sterile water are combined, then the resulting solution comprises:
(a) about 11.23, 22.45, or 56.12 mg/ml sucrose;
(b) about 1.74 mg/ml L-histidine;
(c) about 59.11 mg/ml L-arginine-HCl;
(d) about 0.62 mg/ml calcium chloride; and
(e) about 1.12 mg/ml poloxamer 188.

In some embodiments, when the lyophilized pharmaceutical composition and the sterile water are combined, then the resulting solution comprises:
(a) about 11.23, 22.45, or 56.12 mg/ml sucrose;
(b) about 1.74 mg/ml L-histidine;
(c) about 48.88 mg/ml L-arginine;
(d) about 0.82 mg/ml calcium chloride dihydrate; and
(e) about 1.12 mg/ml poloxamer 188.

In some embodiments, when the lyophilized pharmaceutical composition and the sterile water are combined, then the resulting solution comprises:
(a) about 11.23, 22.45, or 56.12 mg/ml sucrose;
(b) about 1.74 mg/ml L-histidine;
(c) about 48.88 mg/ml L-arginine;
(d) about 0.62 mg/ml calcium chloride; and
(e) about 1.12 mg/ml poloxamer 188.

In some embodiments, when the lyophilized pharmaceutical composition and the sterile water are combined, then the resulting solution comprises:
(a) about 33.67, 67.34, or 168.35 mg sucrose;
(b) about 5.23 mg L-histidine;
(c) about 177.32 mg L-arginine-HCl;
(d) about 2.47 mg calcium chloride; and
(e) about 3.37 mg poloxamer 188 in 3 mL of sterile water.

In some embodiments, when the lyophilized pharmaceutical composition and the sterile water are combined, then the resulting solution comprises:
(a) about 33.67, 67.34, or 168.35 mg sucrose;
(b) about 5.23 mg L-histidine;
(c) about 146.63 mg L-arginine;
(d) about 2.47 mg calcium chloride dihydrate; and
(e) about 3.37 mg poloxamer 188 in 3 mL of sterile water.

In some embodiments, when the lyophilized pharmaceutical composition and the sterile water are combined, then the resulting solution comprises:
(a) about 33.67, 67.34, or 168.35 mg sucrose;
(b) about 5.23 mg L-histidine;
(c) about 146.63 mg L-arginine;
(d) about 1.87 mg calcium chloride; and
(e) about 3.37 mg poloxamer 188 in 3 mL of sterile water.

In some embodiments, when the lyophilized pharmaceutical composition and the sterile water are combined, then the resulting solution comprises:
(a) 33.67, 67.34, or 168.35 mg sucrose;
(b) 5.23 mg L-histidine;
(c) 177.32 mg L-arginine-HCl;
(d) 2.47 mg calcium chloride dihydrate; and
(e) 3.37 mg poloxamer 188 in 3 mL of sterile water.

In some embodiments, when the lyophilized pharmaceutical composition and the sterile water are combined, then the resulting solution comprises:
(a) 33.67, 67.34, or 168.35 mg sucrose;
(b) 5.23 mg L-histidine;
(c) 177.32 mg L-arginine-HCl;
(d) 1.87 mg calcium chloride; and
(e) 3.37 mg poloxamer 188 in 3 mL of sterile water.

In some embodiments, when the lyophilized pharmaceutical composition and the sterile water are combined, then the resulting solution comprises:
(a) 33.67, 67.34, or 168.35 mg sucrose;
(b) 5.23 mg L-histidine;
(c) 146.63 mg L-arginine;
(d) 2.47 mg calcium chloride dihydrate; and
(e) 3.37 mg poloxamer 188 in 3 mL of sterile water.

In some embodiments, when the lyophilized pharmaceutical composition and the sterile water are combined, then the resulting solution comprises:
(a) 33.67, 67.34, or 168.35 mg sucrose;
(b) 5.23 mg L-histidine;
(c) 146.63 mg L-arginine;
(d) 1.87 mg calcium chloride; and
(e) 3.37 mg poloxamer 188 in 3 mL of sterile water.

In some embodiments, when the lyophilized pharmaceutical composition and the sterile water are combined, then the lyophilized pharmaceutical composition is reconstituted within 7 to 12 seconds.

In some embodiments, when the lyophilized pharmaceutical composition and the sterile water are combined, then the osmolality of the resulting solution is about 525 to about 725 mOsm/kg. In some embodiments, the pharmaceutical composition disclosed herein has an osmolality about 500 to about 650 mOsm/kg. In some embodiments, when the lyophilized pharmaceutical composition and the sterile water are combined, then the osmolality of the resulting solution is about 600 to about 650 mOsm/kg.

In some embodiments, the pharmaceutical composition has an osmolality of about 525 mOsm/kg. In some embodiments, the pharmaceutical composition has an osmolality of about 550 mOsm/kg. In some embodiments, the pharmaceutical composition has an osmolality of about 575 mOsm/kg. In some embodiments, the pharmaceutical composition has an osmolality of about 600 mOsm/kg. In some embodiments, the pharmaceutical composition has an osmolality of about 625 mOsm/kg. In some embodiments, the pharmaceutical composition has an osmolality of about 650 mOsm/kg. In some embodiments, the pharmaceutical composition has an osmolality of about 675 mOsm/kg. In some embodiments, the pharmaceutical composition has an osmolality of about 700 mOsm/kg. In some embodiments, the pharmaceutical composition has an osmolality of about 725 mOsm/kg.

In some embodiments, when the lyophilized pharmaceutical composition and the sterile water are combined, then the pH of the resulting solution is about 6.5 to about 7.5. In some embodiments, the pharmaceutical composition has a pH of about 7.0. In some embodiments, the pharmaceutical composition has a pH of about 6.8. In some embodiments, the pharmaceutical composition has a pH of 6.8.

In some embodiments, when the lyophilized pharmaceutical composition and the sterile water are combined, then the pH of the resulting solution is about 6.5. In some embodiments, when the lyophilized pharmaceutical composition and the sterile water are combined, then the pH of the resulting solution is about 6.6. In some embodiments, when the lyophilized pharmaceutical composition and the sterile water are combined, then the pH of the resulting solution is about 6.7. In some embodiments, when the lyophilized pharmaceutical composition and the sterile water are combined, then the pH of the resulting solution is about 6.8. In some embodiments, when the lyophilized pharmaceutical composition and the sterile water are combined, then the pH of the resulting solution is about 6.9. In some embodiments, when the lyophilized pharmaceutical composition and the sterile water are combined, then the pH of the resulting solution is about 7.0. In some embodiments, when the lyophilized pharmaceutical composition and the sterile water are combined, then the pH of the resulting solution is about 7.1. In some embodiments, when the lyophilized pharmaceutical composition and the sterile water are combined, then the pH of the resulting solution is about 7.2. In some embodiments, when the lyophilized pharmaceutical composition and the sterile water are combined, then the pH of the resulting solution is about 7.3. In some embodiments, when the lyophilized pharmaceutical composition and the sterile water are combined, then the pH of the resulting solution is about 7.4. In some embodiments, when the lyophilized pharmaceutical composition and the sterile water are combined, then the pH of the resulting solution is about 7.5.

In some embodiments, when the lyophilized pharmaceutical composition and the sterile water are combined, then the protein concentration of the resulting solution is about 0.8 to about 1.2 mg/mL.

In some embodiments, when the lyophilized pharmaceutical composition and the sterile water are combined, then the protein concentration of the resulting solution is about 0.8 mg/mL. In some embodiments, when the lyophilized pharmaceutical composition and the sterile water are combined, then the protein concentration of the resulting solution is about 0.9 mg/mL. In some embodiments, when the lyophilized pharmaceutical composition and the sterile water are combined, then the protein concentration of the resulting solution is about 1.0 mg/mL. In some embodiments, when the lyophilized pharmaceutical composition and the sterile water are combined, then the protein concentration of the resulting solution is about 1.1 mg/mL. In some embodiments, when the lyophilized pharmaceutical composition and the sterile water are combined, then the protein concentration of the resulting solution is about 1.2 mg/mL.

In some embodiments, when the lyophilized pharmaceutical composition and the sterile water are combined, then the protein concentration of the resulting solution is 0.8 mg/mL. In some embodiments, when the lyophilized pharmaceutical composition and the sterile water are combined, then the protein concentration of the resulting solution is 0.9 mg/mL. In some embodiments, when the lyophilized pharmaceutical composition and the sterile water are combined, then the protein concentration of the resulting solution is 1.0 mg/mL. In some embodiments, when the lyophilized pharmaceutical composition and the sterile water are combined, then the protein concentration of the resulting solution is 1.1 mg/mL. In some embodiments, when the lyophilized pharmaceutical composition and the sterile water are combined, then the protein concentration of the resulting solution is 1.2 mg/mL.

In some embodiments, when the lyophilized pharmaceutical composition and the sterile water are combined, then the turbidity of the resulting solution is less than about 7 Nephelometric Turbidity Units.

In some embodiments, when the lyophilized pharmaceutical composition and the sterile water are combined, less than 3% of the chimeric protein is aggregated.

Disclosed herein is a method of storing a pharmaceutical composition, comprising maintaining a pharmaceutical composition disclosed herein at a temperature of from about −20° C. to about −40° C. for at least about 1 to about 36 months. In some embodiments, the pharmaceutical composition is stored at a temperature of about −20° C., about −25° C., about −30° C., about −35° C., or about −40° C. for at least about 1 to about 36 months. In some embodiments, the pharmaceutical composition is stored at a temperature of about −20° C., about −25° C., about −30° C., about −35° C., or about −40° C. for at least about 3, 6, 9, 12, 18, 21, 24, 27, 30, 33, or 36 months. In some embodiments, the pharmaceutical composition is stored at a temperature of about −30° C. for at least about 1, 2, 3, 4, 5, 6, 7, 8, 9, 11, or 12 months.

In some embodiments, the pharmaceutical composition has been stored at a temperature of from about −20° C. to about −40° C. for at least about 1 to about 36 months. In some embodiments, the pharmaceutical composition has been stored at a temperature of about −20° C., about −25° C., about −30° C., about −35° C., or about −40° C. for at least about 1 to about 36 months. In some embodiments, the pharmaceutical composition has been stored at a temperature of about −20° C., about −25° C., about −30° C., about −35° C., or about −40° C. for at least about 3, 6, 9, 12, 18, 21, 24, 27, 30, 33, or 36 months. In some embodiments, the pharmaceutical composition has been stored at a temperature of about −30° C. for at least about 1, 2, 3, 4, 5, 6, 7, 8, 9, 11, or 12 months.

In some embodiments, the pharmaceutical composition is a liquid pharmaceutical composition that has been frozen and then thawed 2-5 times. In some embodiments, the pharmaceutical composition is a liquid pharmaceutical composition that has been frozen and then thawed 2 times. In some embodiments, the pharmaceutical composition is a liquid pharmaceutical composition that has been frozen and then thawed 3 times. In some embodiments, the pharmaceutical composition is a liquid pharmaceutical composition that has been frozen and then thawed 4 times. In some embodiments, the pharmaceutical composition is a liquid pharmaceutical composition that has been frozen and then thawed 5 times.

In some embodiments, the pharmaceutical composition comprises about 0.5 mg/ml to about 10 mg/ml of the chimeric protein. In some embodiments, the pharmaceutical composition comprises about 0.5 mg/ml to about 5 mg/ml of the chimeric protein. In some embodiments, the pharmaceutical composition comprises about 0.5 mg/ml to about 2 mg/ml of the chimeric protein. In some embodiments, the pharmaceutical composition comprises about 0.5 mg/ml of the chimeric protein. In some embodiments, the pharmaceutical composition comprises about 1 mg/ml of the chimeric protein. In some embodiments, the pharmaceutical composition comprises about 1.5 mg/ml of the chimeric protein. In some embodiments, the pharmaceutical composition comprises about 2 mg/ml of the chimeric protein.

### V. Pharmaceutical Kits

In some embodiments, the pharmaceutical composition is provided with a second container comprising sterile water. Disclosed herein are pharmaceutical kits which comprise a first container containing the pharmaceutical composition and a second container containing sterile water.

Disclosed herein is a pharmaceutical kit comprising:
(i) a first container comprising a lyophilized pharmaceutical composition comprising
(a) a chimeric protein comprising a first polypeptide chain which comprises a Factor VIII (“FVIII”) protein or a portion thereof and a first immunoglobulin (“Ig”) constant region or a portion thereof, and a second polypeptide chain which comprises a von Willebrand Factor (“VWF”) protein and a second Ig constant region or a portion thereof;
(b) about 10 mg to about 200 mg sucrose;
(c) about 2.5 mg to about 7.5 mg histidine;
(d) about 140 mg to about 200 mg arginine;
(e) about 1.5 mg to about 5 mg calcium chloride; and
(f) about 0.5 mg to about 10 mg poloxamer 188, and
(ii) a second container comprising sterile water.

In some embodiments, the first container comprises 100 IU to 10,000 IU of the chimeric protein.

In some embodiments, the first container comprises 250 IU, 500 IU, 1000 IU, 2000 IU, 3000 IU, or 4,000 IU of the chimeric protein. In some embodiments, the first container comprises 250 IU of the chimeric protein. In some embodiments, the first container comprises 500 IU of the chimeric protein. In some embodiments, the first container comprises 1000 IU of the chimeric protein. In some embodiments, the first container comprises 2000 IU of the chimeric protein. In some embodiments, the first container comprises 3000 IU of the chimeric protein. In some embodiments, the first container comprises 4,000 IU of the chimeric protein. In some embodiments, the first container comprises more than about 250 IU of the chimeric protein. In some embodiments, the first container comprises more than about 300 IU of the chimeric protein. In some embodiments, the first container comprises more than about 500 IU of the chimeric protein. In some embodiments, the first container comprises at least about 500 IU of the chimeric protein.

In some embodiments, the second container comprises about 2 mL to about 5 mL of sterile water. In some embodiments, the second container comprises 2 mL to 5 mL of sterile water. In some embodiments, the second container comprises about 3 mL of sterile water. In some embodiments, the second container comprises 3 mL of sterile water.

In some embodiments, the second container comprises about 2 mL of sterile water. In some embodiments, the second container comprises about 2.1 mL of sterile water. In some embodiments, the second container comprises about 2.2 mL of sterile water. In some embodiments, the second container comprises about 2.3 mL of sterile water. In some embodiments, the second container comprises about 2.4 mL of sterile water. In some embodiments, the second container comprises about 2.5 mL of sterile water. In some embodiments, the second container comprises about 2.6 mL of sterile water. In some embodiments, the second container comprises about 2.7 mL of sterile water. In some embodiments, the second container comprises about 2.8 mL of sterile water. In some embodiments, the second container comprises about 2.9 mL of sterile water. In some embodiments, the second container comprises about 3 mL of sterile water. In some embodiments, the second container comprises about 3.1 mL of sterile water. In some embodiments, the second container comprises about 3.2 mL of sterile water. In some embodiments, the second container comprises about 3.3 mL of sterile water. In some embodiments, the second container comprises about 3.4 mL of sterile water. In some embodiments, the second container comprises about 3.5 mL of sterile water. In some embodiments, the second container comprises about 3.6 mL of sterile water. In some embodiments, the second container comprises about 3.7 mL of sterile water. In some embodiments, the second container comprises about 3.8 mL of sterile water. In some embodiments, the second container comprises about 3.9 mL of sterile water. In some embodiments, the second container comprises about 4 mL of sterile water. In some embodiments, the second container comprises about 4.1 mL of sterile water. In some embodiments, the second container comprises about 4.2 mL of sterile water. In some embodiments, the second container comprises about 4.3 mL of sterile water. In some embodiments, the second container comprises about 4.4 mL of sterile water. In some embodiments, the second container comprises about 4.5 mL of sterile water. In some embodiments, the second container comprises about 4.6 mL of sterile water. In some embodiments, the second container comprises about 4.7 mL of sterile water. In some embodiments, the second container comprises about 4.8 mL of sterile water. In some embodiments, the second container comprises about 4.9 mL of sterile water. In some embodiments, the second container comprises about 5 mL of sterile water.

In some embodiments, the pharmaceutical kit further comprises instructions for combining the lyophilized pharmaceutical composition and the sterile water.

In some embodiments, the first container is a glass vial comprising a rubber stopper.

In some embodiments, the second container is a syringe body. In some embodiments, the syringe body is associated with a plunger. In some embodiments, the pharmaceutical kit further comprises an adaptor to connect the glass vial to the syringe body. In some embodiments, the pharmaceutical kit further comprises infusion tubing associated with a needle to be connected to the syringe body, suitable for intravenous infusion. In some embodiments, the second container is a pre-filled syringe.

Disclosed herein is a method of storing a pharmaceutical kit, comprising maintaining a pharmaceutical kit disclosed herein at a temperature of from about −20° C. to about −40° C. for at least about 1 to about 36 months. In some embodiments, the pharmaceutical kit is stored at a temperature of about −20° C., about −25° C., about −30° C., about −35° C., or about-40° C. for at least about 1 to about 36 months. In some embodiments, the pharmaceutical kit is stored at a temperature of about −20° C., about −25° C., about −30° C., about −35° C., or about −40° C. for at least about 3, 6, 9, 12, 18, 21, 24, 27, 30, 33, or 36 months. In some embodiments, the pharmaceutical kit is stored at a temperature of about −30° C. for at least about 1, 2, 3, 4, 5, 6, 7, 8, 9, 11, or 12 months.

In some embodiments, the pharmaceutical kit has been stored at a temperature of from about −20° C. to about −40° C. for at least about 1 to about 36 months. In some embodiments, the pharmaceutical kit has been stored at a temperature of about −20° C., about −25° C., about −30° C., about −35° C., or about −40° C. for at least about 1 to about 36 months. In some embodiments, the pharmaceutical kit has been stored at a temperature of about −20° C., about −25° C., about −30° C., about −35° C., or about −40° C. for at least about 3, 6, 9, 12, 18, 21, 24, 27, 30, 33, or 36 months. In some embodiments, the pharmaceutical kit has been stored at a temperature of about −30° C. for at least about 1, 2, 3, 4, 5, 6, 7, 8, 9, 11, or 12 months.

### VI. Methods and Uses of the Pharmaceutical Composition

Also disclosed herein is a use of the pharmaceutical composition or a method for treating hemophilia A in a subject in need thereof, comprising administering to the subject an effective amount of the pharmaceutical composition of the disclosure. In some embodiments, the treatment of hemophilia A comprises preventing a bleeding episode in a human subject in need thereof. In some embodiments, the treatment of hemophilia A comprises treating a bleeding episode in a human subject in need thereof. In some embodiments, the treatment of hemophilia A comprises controlling the incidence or frequency of a bleeding episode in a human subject in need thereof. In some embodiments, the treatment of hemophilia A comprises decreasing the incidence or frequency of a bleeding episode in a human subject in need thereof.

In some embodiments, the composition is used to treat a bleeding disease or condition in a subject in need thereof. The bleeding disease or condition is selected from the group consisting of a bleeding coagulation disorder, hemarthrosis, muscle bleed, oral bleed, hemorrhage, hemorrhage into muscles, oral hemorrhage, trauma, trauma capitis, gastrointestinal bleeding, intracranial hemorrhage, intra-abdominal hemorrhage, intrathoracic hemorrhage, bone fracture, central nervous system bleeding, bleeding in the retropharyngeal space, bleeding in the retroperitoneal space, bleeding in the illiopsoas sheath and any combinations thereof. In still other embodiments, the subject is scheduled to undergo a surgery. In some embodiments, the treatment is prophylactic or on-demand.

In some embodiments, the use or method comprises combining the lyophilized pharmaceutical composition and the sterile water of a kit of the disclosure, and administering to the subject an effective amount of the resulting combination. In some embodiments, the subject combines the lyophilized pharmaceutical composition and the sterile water of the kit. In some embodiments, the combination is self-administered by the subject.

In some embodiments, the multiple doses comprise at least two doses, at least three doses, at least four doses, at least five doses, at least six doses, at least seven doses, at least eight doses, at least nine doses, at least ten doses, at least eleven doses, at least twelve doses, at least thirteen doses, at least fourteen doses, at least fifteen doses, at least sixteen doses, at least seventeen doses, at least eighteen doses, at least nineteen doses, at least twenty doses, or more. In some embodiments, the multiple doses are administered for at least about 1 month, at least about 2 months, at least about 3 months, at least about 4 months, at least about 5 months, at least about 6 months, at least about 12 months, at least about 18 months, at least about 2 years, at least about 3 years, at least about 4 years, at least about 5 years, at least about 10 years, at least about 15 years, at least about 20 years, or for at least about 25 years.

A chimeric protein described herein can be administered by any means known in the art. In some embodiments, the chimeric protein is administered by a route selected from the group consisting of intravenous injection, intravenous infusion, subcutaneous administration, intramuscular administration, oral administration, nasal administration, and pulmonary administration. In some embodiments, the chimeric protein is administered intravenously. In some embodiments, the chimeric protein is administered subcutaneously.

Having now described the present disclosure in detail, the same will be more clearly understood by reference to the following examples, which are included herewith for purposes of illustration only and are not intended to be limiting of the disclosure. All patents, publications, and articles referred to herein are expressly and specifically incorporated herein by reference.

### EXAMPLES

### Example 1: Evaluation of Aggregation of Efanesoctocog Alfa Compositions Comprising Different Surfactants

The experiments described in this example assess the stability of efanesoctocog alfa in pharmaceutical compositions comprising a different surfactant (poloxamer 188) formulated with different concentrations of sucrose. In addition to sucrose, each composition also contained 10 mM L-histidine, 250 mM L-arginine-HCl, 5 mM CaCl₂), and 0.1% (w/v) poloxamer 188 (poloxamer 188), at a pH of 6.8. Stability attributes for poloxamer 188 compositions were also compared to stability attributes for otherwise identical PS80 compositions to evaluate the effect of selected surfactant.

To further analyze the impact on sucrose concentration on efanesoctocog alfa compositions containing poloxamer 188, pharmaceutical compositions of efanesoctocog alfa at 0, 1, 2, and 5% w/v sucrose were subjected to different stability conditions and stability was assessed using several methods. Compositions were tested at the following product stages: drug substance (DS, which was had not been lyophilized or adjusted for any particular IU strength) frozen storage stability, DS freeze/thaw (FT) stability, liquid DS post-thaw stability, diluted DS and drug product (DP) stability, DP lyophilization stability, and DP-post reconstitution.

Aggregation of efanesoctocog alfa compositions was measured by SEC assay (% HMWS). The SEC assay uses an HPLC instrument equipped with a pump, a temperature controlled autosampler, a column heater, and fluorescence detector. The sample solutions are analyzed using the following instrumentation and method parameters:
Mobile Phase: Dulbecco's Phosphate-Buffered Saline (D-PBS) containing Calcium and Magnesium adding 0.36 M Sodium Chloride (0.9 mM Calcium Chloride, 0.5 mM Magnesium Chloride, 2.7 mM Potassium Chloride, 1.5 mM Potassium Phosphate Mono basic, 496 mM Sodium Chloride, 8.1 mM Sodium Phosphate Dibasic, pH 7. 0±0.1)
Column Heater: 26° C. . . .
Run Time: 40 minutes
FL Detector: Ex/Em=280/350 nm, PMT Gain=5
Injection Mass Load: 2 μg
Autosampler Temperature: 5° C.
Flow Rate: 0.5 mL/min, Isocratic

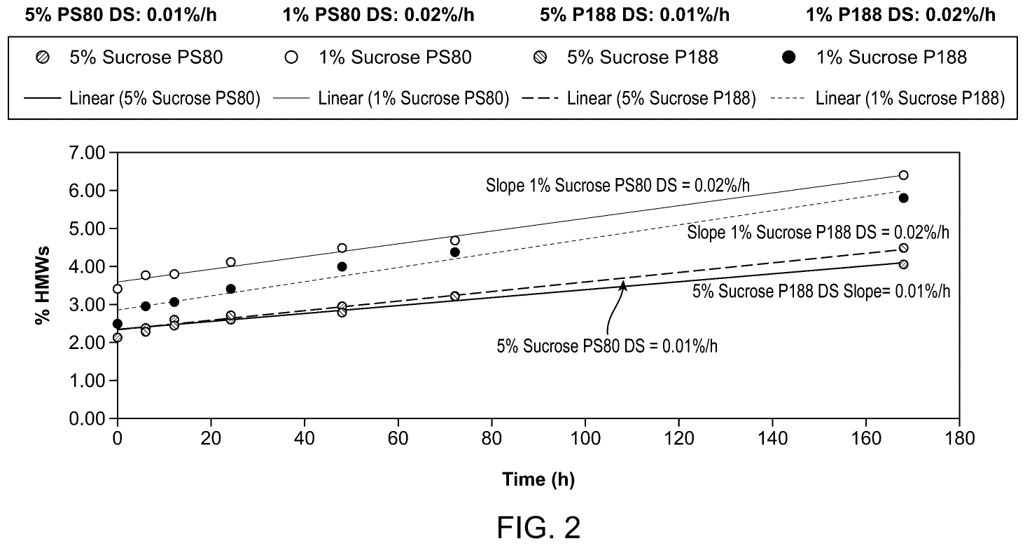

Aggregation of efanesoctocog alfa drug substance (DS) compositions (1 mg/ml) containing either 0.5% (w/v) PS80 or 0.1% (w/v) poloxamer 188 as a surfactant and and either 1% or 5% (w/v) sucrose were tested at room temperature (RT)/room light (RL) conditions or at 2-8° C. Aggregation results for RT/RL conditions over time (hours) are shown in FIG. 2. At RT/RL conditions, reduction of sucrose from 5% to 1% increased aggregation by 2× for compositions containing either surfactant. At RT/RL conditions, aggregation rate increases were about the same for both poloxamer 188 compositions and PS80 compositions.

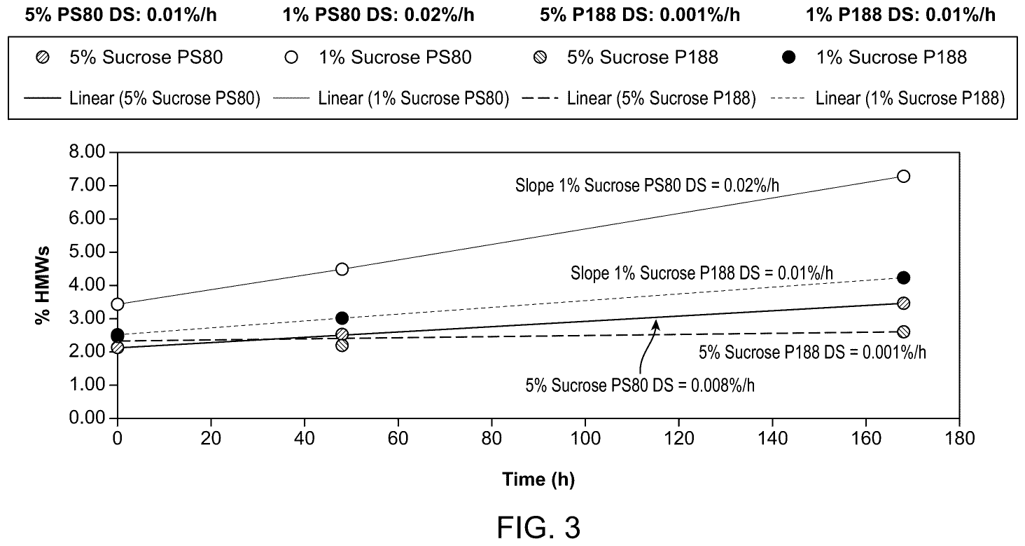

Aggregation results at 2-8° C. over time (hours) are shown in FIG. 3. At 2-8° C. conditions, reduction of sucrose from 5% to 1% increased aggregation by 2× in PS80 compositions, which was similar to the results at RT/RL conditions. However, at 2-8° C., reduction of sucrose from 5% to 1% increased aggregation by 10× for PS80 compositions. At 2-8° C., aggregation rate increases were lower in poloxamer 188 compositions than PS80 compositions.

Aggregation of efanesoctocog alfa drug substance (DS) liquid compositions at 4000 IU and containing either 0.5% (w/v) PS80 or 0.1% (w/v) poloxamer 188 as a surfactant were assessed in a cumulative hold time assay. Efanesoctocog alfa liquid compositions were held at RT and tested after 0, 5, 7, 25, 43, and 55 hours. Samples were also tested after lyophilization.

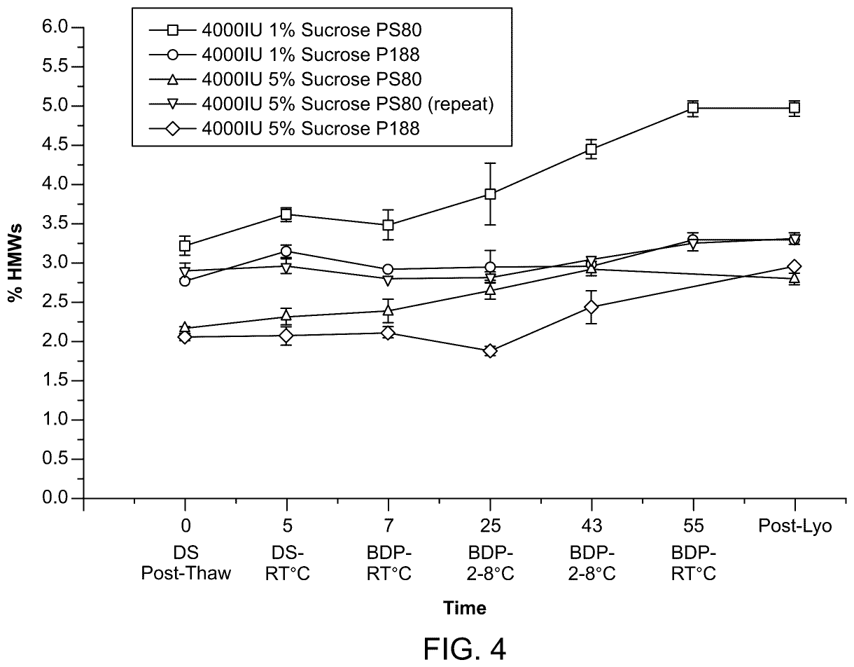

Results of the cumulative hold time assay are shown in FIG. 4. Efanesoctocog alfa drug substance (DS) liquid compositions followed by subsequent dilutions to 4000 IU comprising 1% sucrose and 0.05% PS80 showed the highest increase in aggregation (see FIG. 4, square plot). The results of this cumulative hold time assay also show that compositions comprising poloxamer 188 may be moderately more stable than PS80 compositions in liquid state at RT.

Frozen storage stability of compositions of efanesoctocog alfa DS at 1 mg/ml was assessed for compositions comprising either 0.5% (w/v) PS80 or 0.1% (w/v) poloxamer 188 as a surfactant. Compositions containing 0, 1, 2, or 5% (w/v) sucrose were held at freezing temperatures (−80° C. or −30° C.). Time points measured were pre-freeze, start of experiment (TO), one month (T1M), 3 months (T3M), and 6 months (T6M).

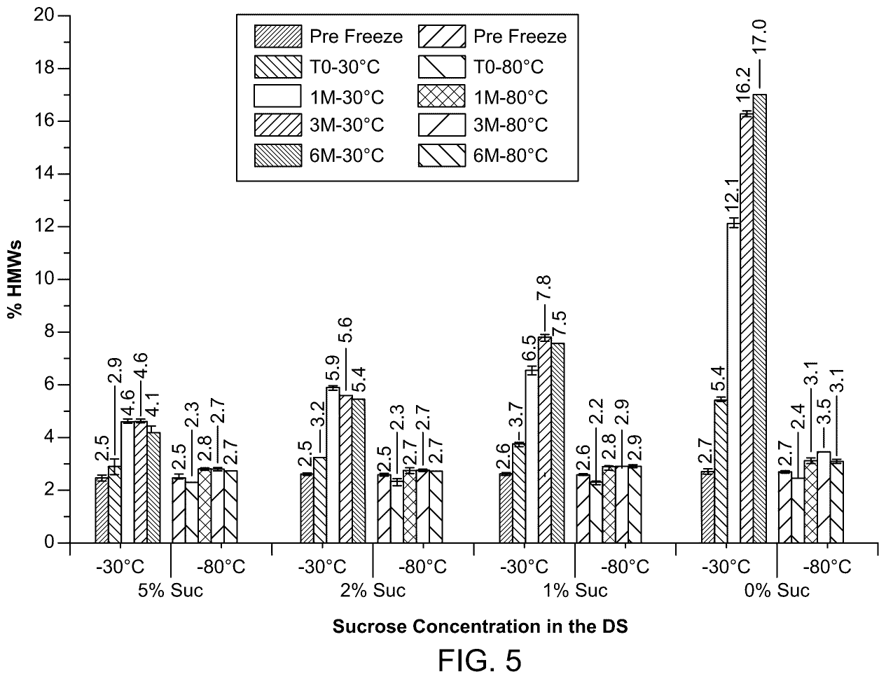

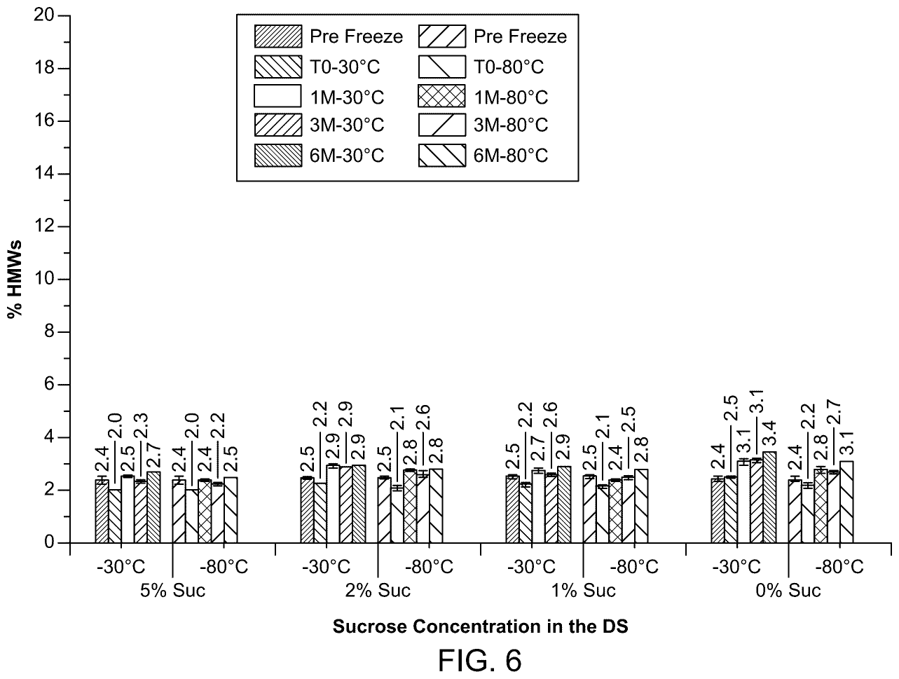

Results of the frozen storage stability assay for compositions comprising 0.5% (w/v) PS80 are shown in FIG. 5. Results of the frozen storage stability assay for compositions comprising 0.1% (w/v) poloxamer 188 are shown in FIG. 6. No trend in aggregation was observed for any of the tested compositions at −80° C. However, at at −30° C., compositions comprising PS80 surfactant showed an increase in aggregation (see FIG. 5). This effect was most evident at lower sucrose concentrations (see, e.g., FIG. 5, 0% sucrose result). In contrast, compositions comprising poloxamer 188 did not show an increase in aggregation at −30° C. (see FIG. 6). Compositions comprising poloxamer 188 showed comparable aggregation levels at both −80° C. and −30° C. Thus, use of poloxamer 188 as a surfactant may provide improved frozen storage stability for efanesoctocog alfa compositions, particularly if compositions are stored at −30° C.

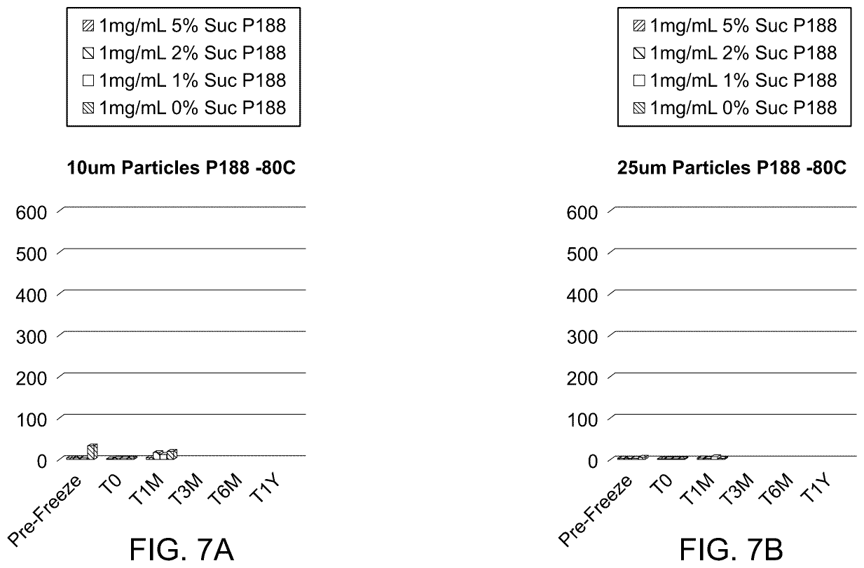

The frozen storage stability of efanesoctocog alfa DS compositions (1 mg/mL) comprising 0.1% (w/v) poloxamer 188 was further analyzed by particle testing using high accuracy liquid particle counter (HIAC). DS compositions containing 0.1% (w/v) poloxamer 188 and 0, 1, 2, or 5% (w/v) sucrose were held at −80° C. and assessed at the following time points: pre-freeze, start of experiment (TO), one month (T1M), 3 months (T3M), and 6 months (T6M). Results for ≥10 μm particles are shown in FIG. 7A. Results for ≥25 μm particles are shown in FIG. 7B. The ≥10 μm particles are less than 6000 particles and ≥25 μm particles are less than 600 particles, and within the limit of <USP 787>. No significant trending in particles was observed over storage.

Aggregation of compositions of efanesoctocog alfa lyophilized drug product (Lyo DP) at 250 IU or 4000 IU and containing 0.1% (w/v) poloxamer 188 were tested at 5° C., 30° C., and 40° C. Lyo DP compositions containing 1% and 5% w/v sucrose were tested. Samples were tested at TO, 1 month, 2 months, 3 months, and 6 months.

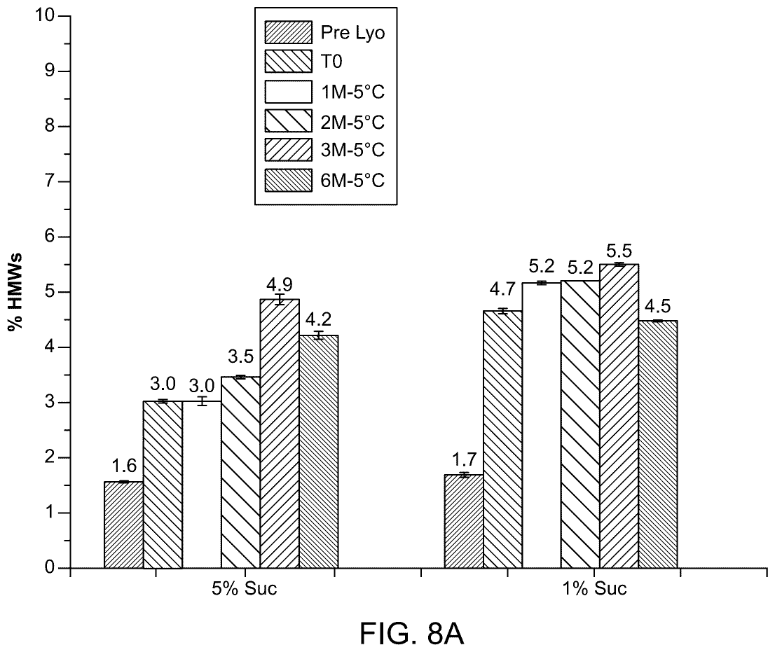

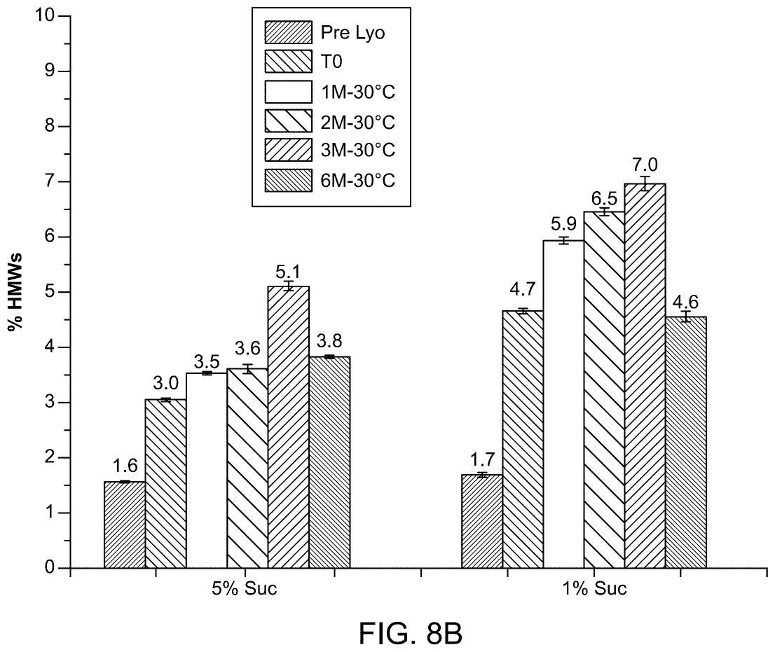

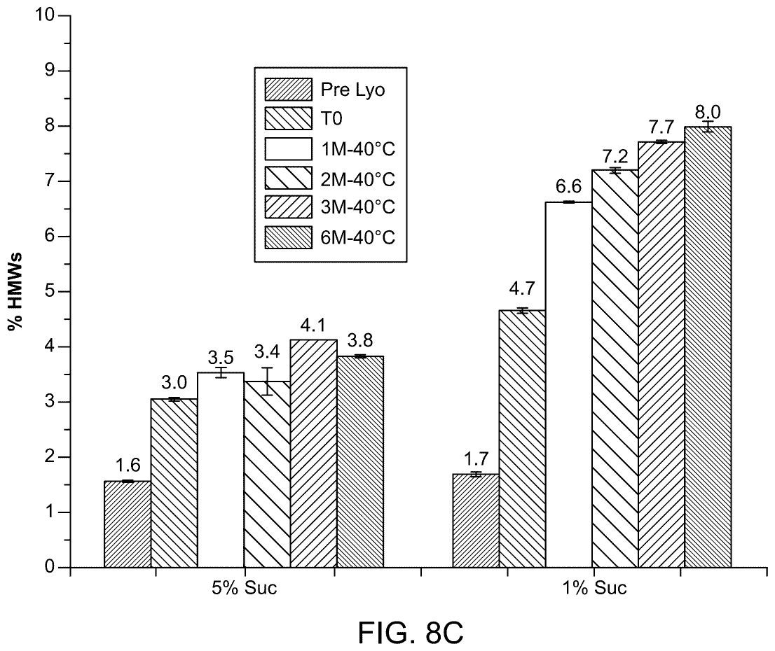

Results of Lyo DP compositions with at 250 IU are shown in FIG. 8A (5° C.), FIG. 8B (30° C.), and FIG. 8C (40° C.). For the 250 IU compositions, higher aggregation was generally demonstrated in poloxamer 188 compositions than PS80 compositions (data not shown). The 250 IU compositions comprising 0.1% (w/v) poloxamer 188 and 1% sucrose demonstrated significant increases in aggregation at all temperatures.

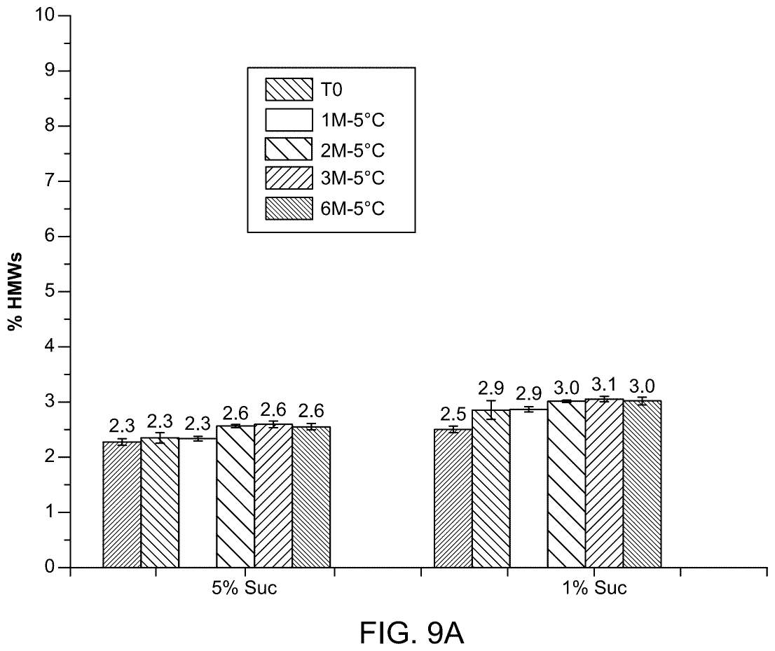

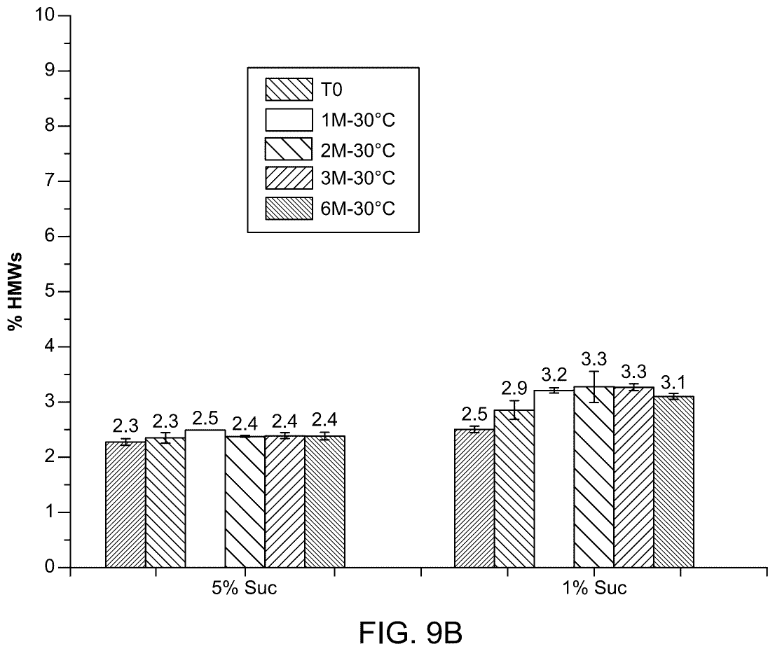

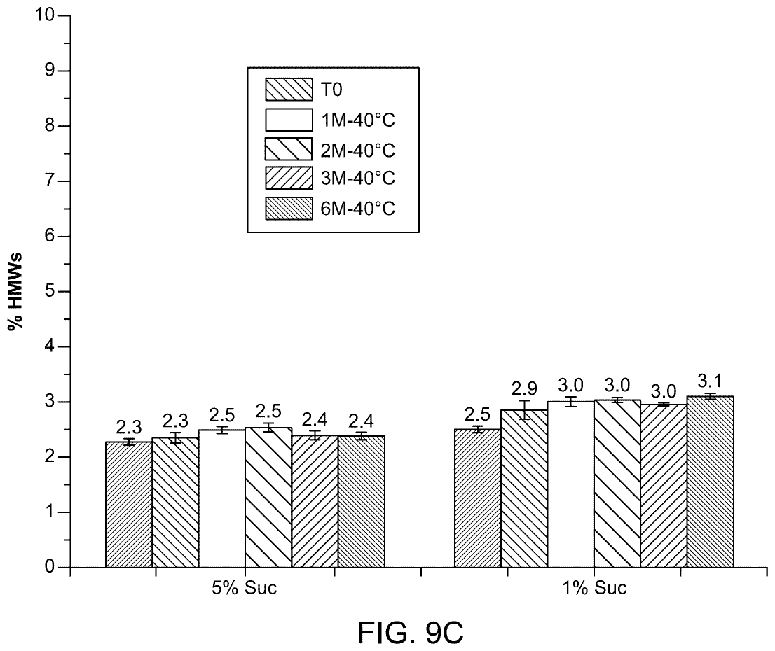

Results of Lyo DP compositions at 4000 IU are shown in FIG. 9A (5° C.), FIG. 9B (30° C.), and FIG. 9C (40° C.). For all 4000 IU compositions comprising 0.1% (w/v) poloxamer 188, no aggregation trends were observed at any temperature for both sucrose concentrations. This result is consistent with results for Lyo DP compositions at 4000 IU comprising 0.5% (w/v) PS80, which also did not demonstrate notable aggregation trends based on sucrose concentration or temperature (data not shown).

Aggregation of efanesoctocog alfa compositions following freeze/thaw (F/T) stress was also analyzed for each surfactant. Compositions of efanesoctocog alfa DS at 1 mg/ml and comprising either 0.5% (w/v) PS80 or 0.1% (w/v) poloxamer 188 as surfactant and 0, 1, 2, or 5% (w/v) sucrose were subjected to stress by F/T at either −80° C. or −30° C. for 5 cycles (5×) (not less than 24 hrs, thaw at room temperature). Samples were tested before freezing (Pre Freeze) and after 5×F/T cycles.

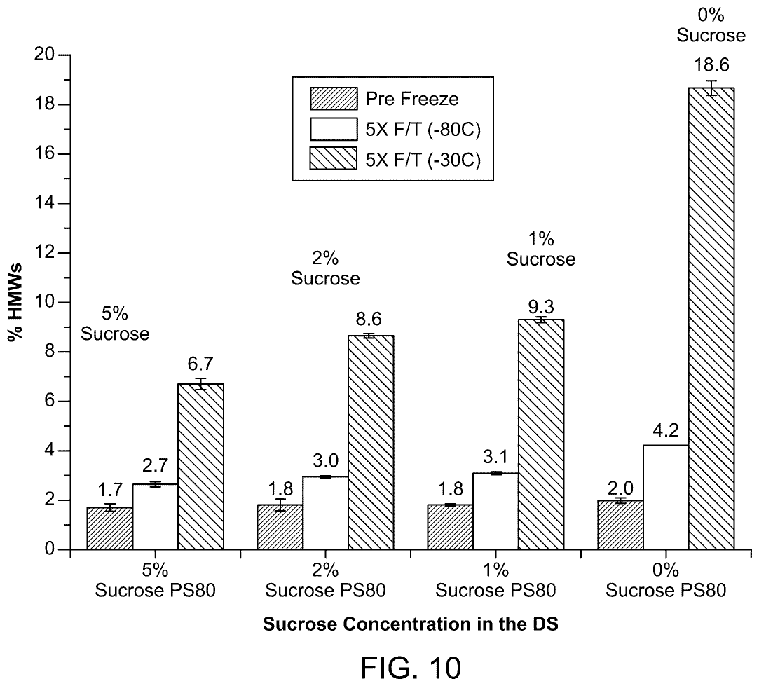

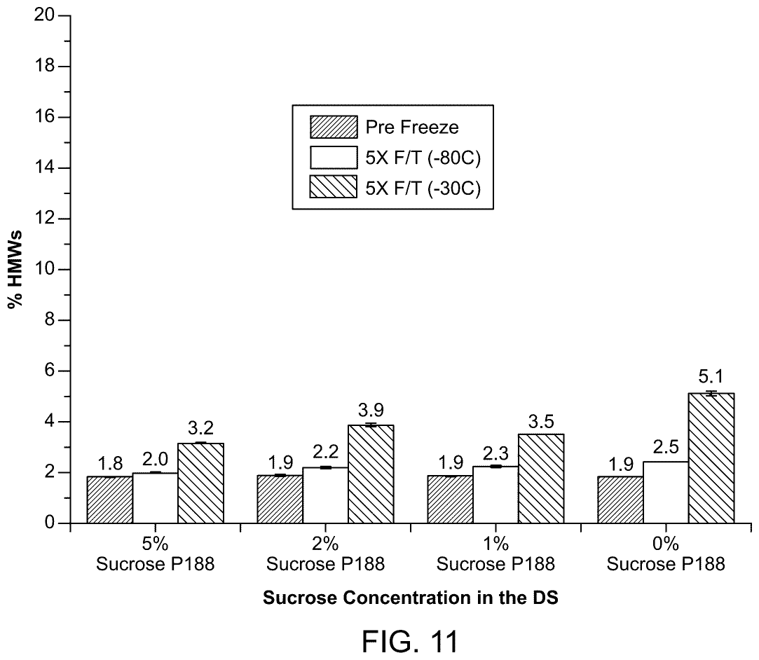

Results for compositions comprising 0.5% (w/v) PS80 are shown in FIG. 10. Results for compositions comprising 0.1% (w/v) poloxamer 188 are shown in FIG. 11. DS compositions comprising 0.5% (w/v) PS80 showed a moderate increase in aggregation after 5×F/T cycles at −80° C. (see FIG. 10). However, DS compositions comprising 0.1% (w/v) poloxamer 188 showed no change in aggregation after 5×F/T cycles at −80° C. (see FIG. 11). Compositions comprising either surfactant showed increases in aggregation after 5×F/T cycles at −30° C. However, the increase in aggregation was significantly greater in DS compositions comprising 0.5% (w/v) PS80 than compositions comprising 0.1% (w/v) poloxamer 188 (compare FIG. 10 and FIG. 11). Thus, it appears that 0.1% (w/v) poloxamer 188 as a surfactant in efanesoctocog alfa compositions may provide slightly improved F/T stability at −80° C. and significantly improved F/T stability at −30° C. over 0.5% (w/v) PS80 as surfactant.

A summary of the results of the aggregation testing for efanesoctocog alfa compositions comprising 0.1% (w/v) poloxamer 188 is provided in Table 1.

TABLE 1
Summary of aggregation analysis (% HMWS) for 0.1% (w/v) poloxamer 188 formulations.

| | | | Liquid DS | | | | | | | | | |
|---|---|---|---|---|---|---|---|---|---|---|---|---|
| | Frozen Storage, % | | Rate %/h | Liquid BDP Rate %/ | | Cum. %/h | Lyo DP (Δ6M) | | | Lyo DP (Δ6M) | | |
| Sucrose | (−80° C.) (1 mg/mL) | | (1 mg/mL) | h (4000 IU) | | (4000 IU) | (4000 IU) | | | (250 IU) | | |
| conc, % | Δ6M | Δ5XFT | RT | 5° C. | RT | 5° C./RT | 5° C. | 30° C. | 40° C. | 5° C. | 30° C. | 40° C. |
| 5 | 0.5 | 0.2 | 0.00 | 0.00 | 0.05 | 0.02 | 0.2 | 0.0 | 0.0 | 1.2 | 0.8 | −0.3 |
| 2 | 0.7 | 1.3 | ND | ND | ND | ND | ND | ND | ND | ND | ND | ND |
| 1 | 0.3 | 1.3 | 0.08 | 0.03 | 0.04 | 0.03 | 0.2 | 0.3 | −0.1 | −0.2 | −0.1 | 3.34 |
| 0 | 0.7 | 0.4 | ND | ND | ND | ND | ND | ND | ND | ND | ND | ND |

ND = Not Determined

Overall, these aggregation studies suggest that efanesoctocog alfa compositions may have improved stability when formulated with 0.1% (w/v) poloxamer 188 rather than 0.5% (w/v) PS80. poloxamer 188 compositions showed a significant improvement over PS80 compositions in F/T and Frozen Storage Stability assays at −30° C. across all sucrose concentrations. Accordingly, poloxamer 188 may be preferable to PS80 for any efanesoctocog alfa compositions to be stored at −30° C. Efanesoctocog alfa DS and BDP liquid compositions also showed improved stability with poloxamer 188 compositions over PS80 compositions. For compositions of efanesoctocog alfa lyophilized DP, poloxamer 188 compositions at 4000 IU showed favorable stability over 6 months at 40° C. However, PS80 lyophilized drug product showed significantly increased stability at 250 IU as compared to poloxamer 188.

### Example 2: Evaluation of Additional Stability Attributes for Efanesoctocog Alfa Compositions Comprising Different Surfactants

In addition to aggregation, other measures of stability of efanesoctocog alfa compositions comprising different surfactants were evaluated.

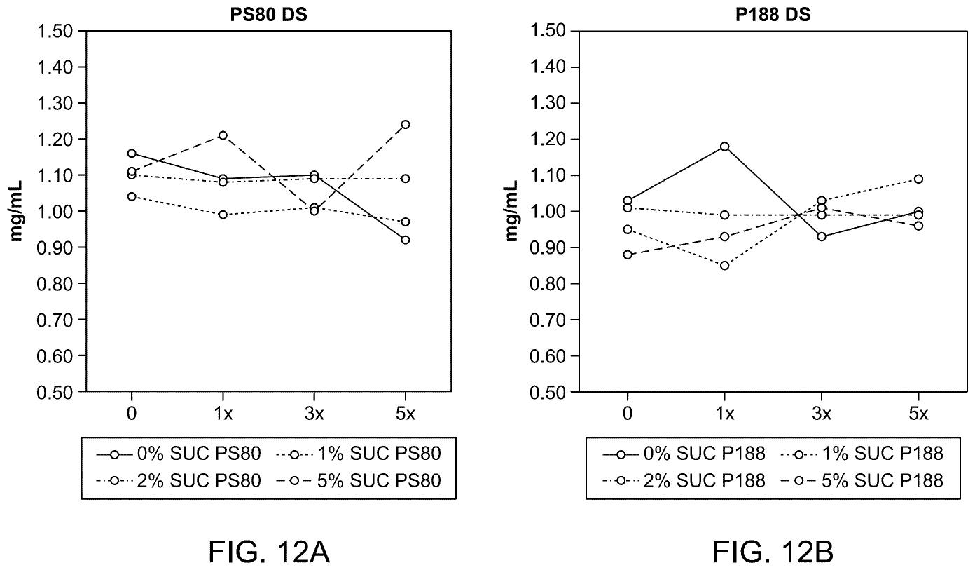

Concentration of surfactant in efanesoctocog alfa compositions following F/T stress was analyzed by high performance liquid chromatography (HPLC). Compositions of efanesoctocog alfa DS at 1 mg/mL and comprising either 0.5% (w/v) PS80 or 0.1% (w/V) poloxamer 188 as surfactant and 0, 1, 2, or 5% (w/v) sucrose were subjected to stress by F/T for 5 F/T cycles at either −80° C. or −30° C. (not less than 24 hrs, thaw at room temperature). Samples were tested before freezing (Pre Freeze) and after 1, 3, or 5 F/T cycles. Results for DS compositions containing 0.5% (w/v) PS80 and 0, 1, 2, or 5% (w/V) sucrose are shown in FIG. 12A. Results for DS compositions containing 0.1% (w/V) poloxamer 188 and 0, 1, 2, or 5% (w/v) sucrose are shown in FIG. 12B. No change in surfactant concentration was observed over all F/T cycles for either surfactant or sucrose concentration.

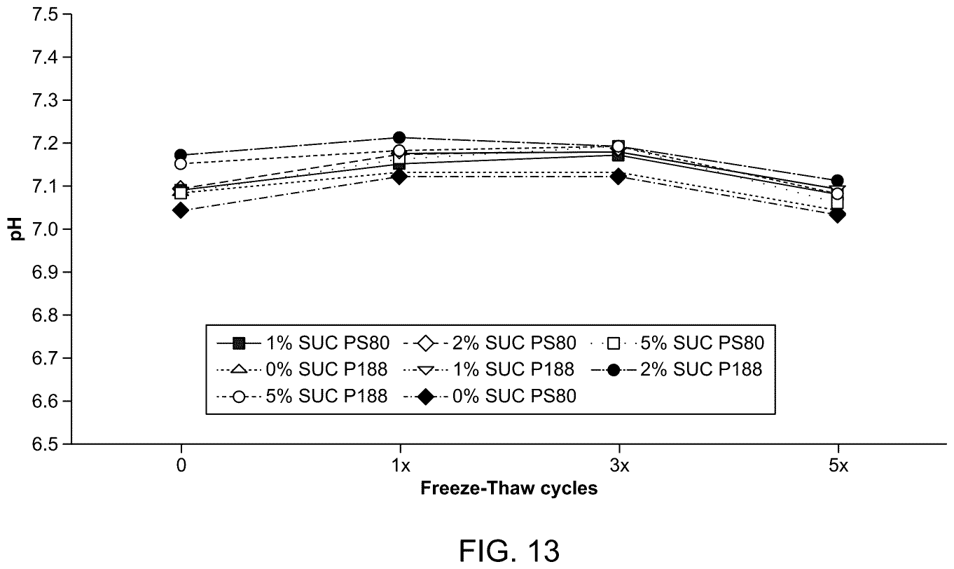

Change in pH of efanesoctocog alfa compositions following F/T stress was also analyzed. Compositions of efanesoctocog alfa DS at 1 mg/mL and comprising either 0.5% (w/V) PS80 or 0.1% (w/v) poloxamer 188 as surfactant and 0, 1, 2, or 5% (w/v) sucrose were subjected to stress by F/T for 5 F/T cycles at either −80° C. or −30° C. (not less than 24 hrs, thaw at room temperature). Samples were tested before freezing (Pre Freeze) and after 1, 3, or 5 F/T cycles. Results of pH analysis are shown in FIG. 13. No significant pH trends or changes were observed for any composition tested.

Turbidity of compositions comprising efanesoctocog alfa DS at 1 mg/mL and poloxamer 188 were tested. Compositions comprising 1% or 5% sucrose were tested, and the results were compared to the turbidity results of efanesoctocog alfa DS at 1 mg/mL compositions comprising PS80. No trends or changes in turbidity were observed for any of the tested poloxamer 188 compositions (data not shown).

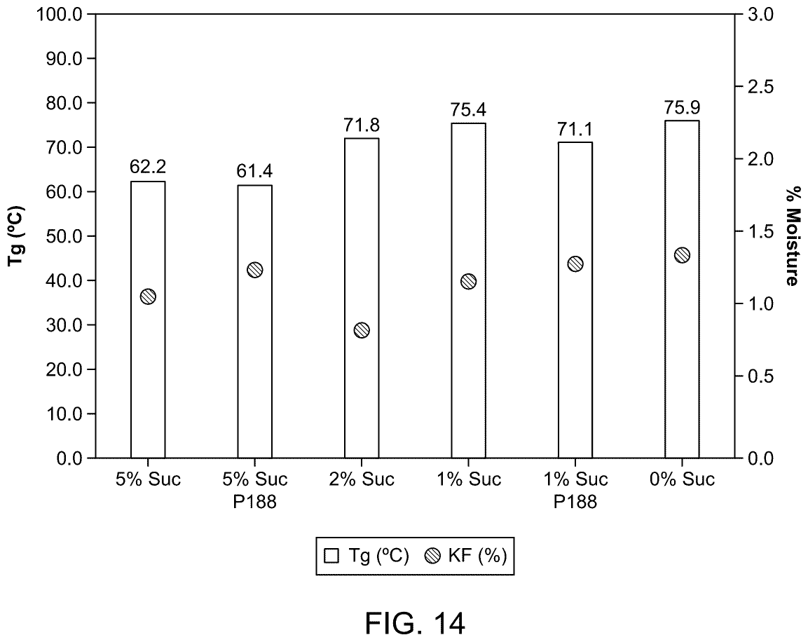

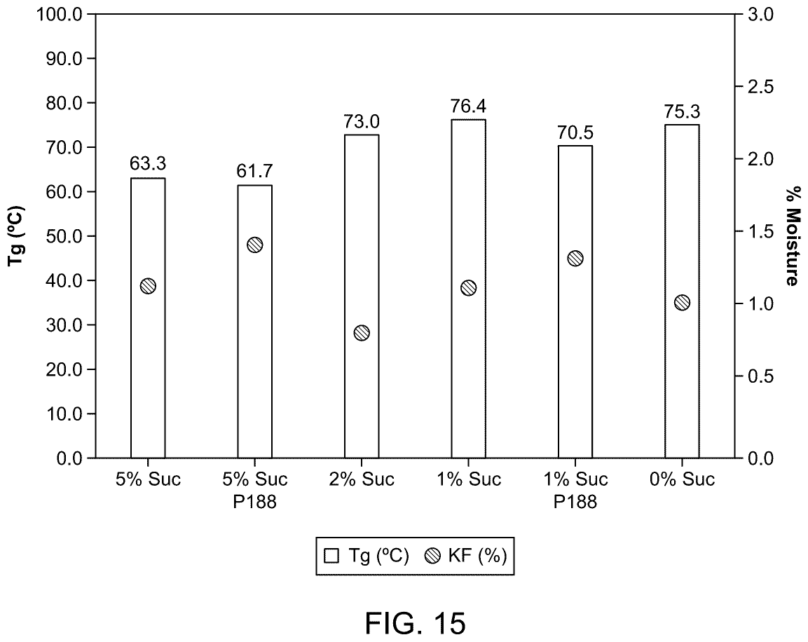

The glass transition temperature (Tg) was measured for efanesoctocog alfa lyophilized drug product (Lyo DP) compositions at 250 IU or 4000 IU and containing 0.1% (w/V) poloxamer 188 as a surfactant and 0, 1, 2, or 5% (w/V) sucrose. Tg was measured by modulated differential scanning calorimetry (DSC), according to the following steps: (i) equilibration at 15° C., (ii) modulate at 1° C. every 60 seconds, (iii) isothermal for 5 minutes; and (iv) ramp at 3° C./min to 130° C. Residual moisture content (%) was also measured for all samples. Tg and residual moisture content results for Lyo DP 250 IU at TO are shown in FIG. 14. Tg and residual moisture content results for Lyo DP 4000 IU at TO are shown in FIG. 15.

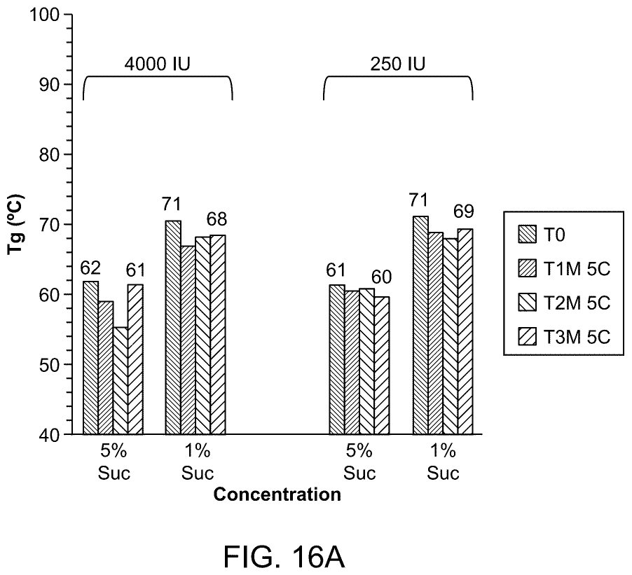

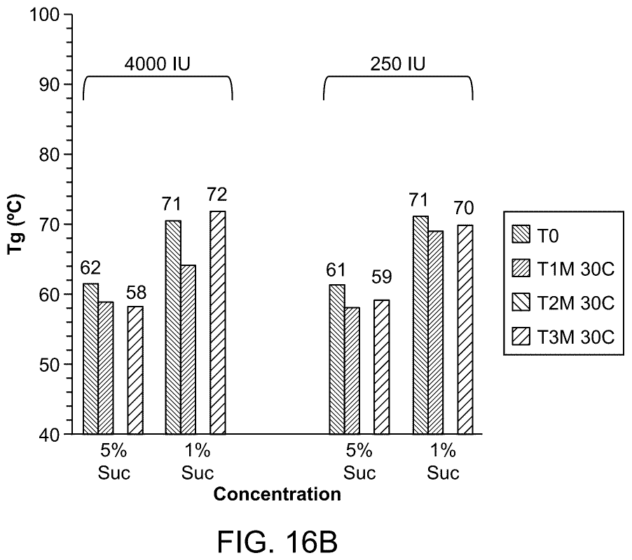

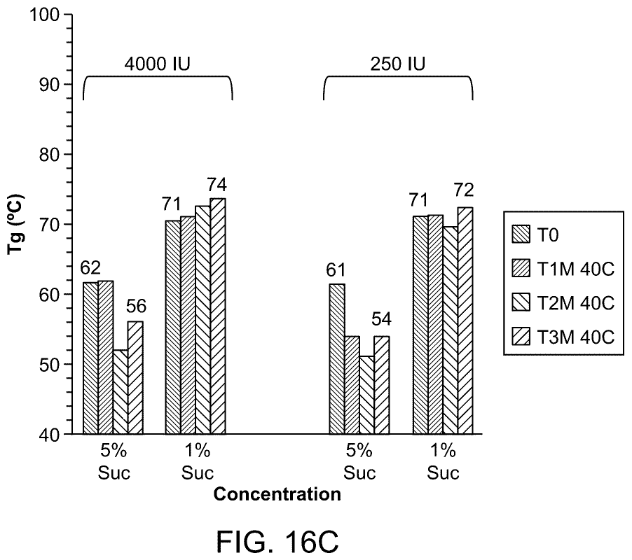

Tg was determined for efanesoctocog alfa lyophilized drug product (Lyo DP) compositions at 4000 IU or 250 IU and containing 0.1% (w/v) poloxamer 188 as a surfactant and 5% or 1% (w/v) sucrose. Samples were held at 5° C., 30° C., or 40° C. and tested at TO, 1 month, 2 months, 3 months, and 6 months. Results are shown in FIG. 16A (5° C.), FIG. 16B (30° C.), and FIG. 16C (40° C.).

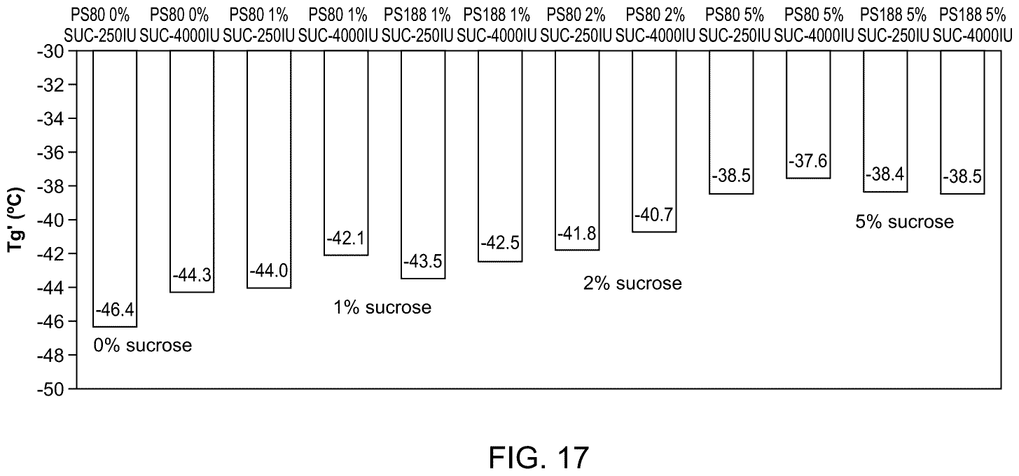

The glass transition temperature (Tg) was also determined for efanesoctocog alfa liquid bulk drug product (BDP) compositions at 250 IU or 4000 IU and containing either 0.5% (w/v) PS80 or 0.1% (w/V) poloxamer 188 as a surfactant and 0, 1, 2, or 5% (w/v) sucrose. Tg results for both surfactants at all strengths and sucrose concentrations are shown in FIG. 17.

For both 250 IU or 4000 IU compositions comprising either surfactant, Tg was generally observed to correlate with sucrose concentration, with the higher sucrose concentration showing lower Tg. No significant differences in Tg were observed based on surfactant type for either 250 IU or 4000 IU concentration.

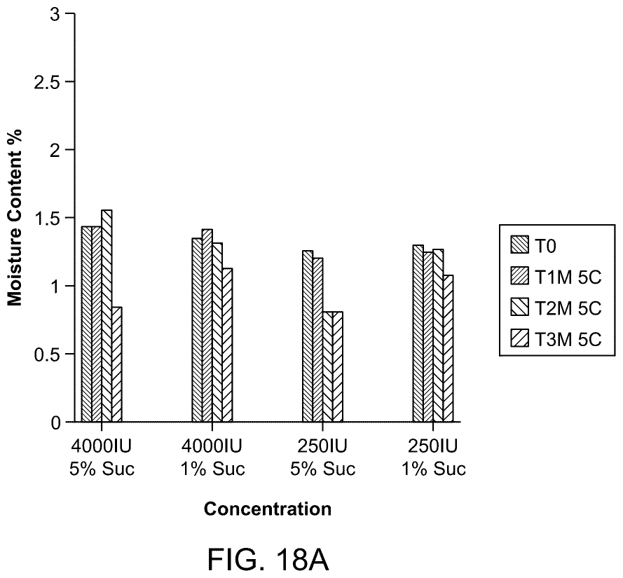

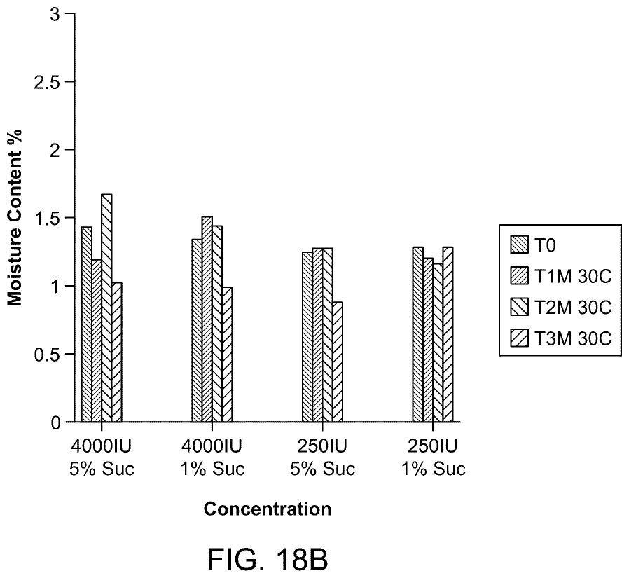

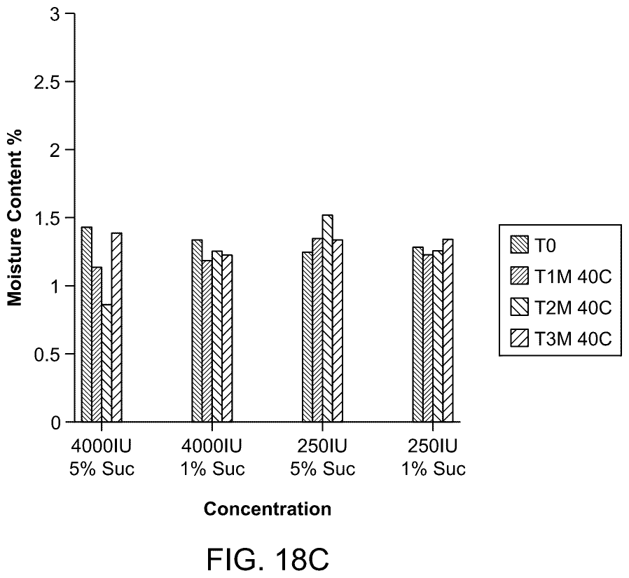

Residual moisture content was determined for efanesoctocog alfa lyophilized drug product (Lyo DP) compositions at 250 IU or 4000 IU and containing 0.1% (w/v) poloxamer 188 as a surfactant and 1% or 5% (w/v) sucrose. Samples were held at 5° C., 30° C., or 40° C. and tested at TO, 1 month, 2 months, 3 months, and 6 months. Results are shown in FIG. 18A (5° C.), FIG. 18B (30° C.), and FIG. 18C (40° C.). No significant change in residual moisture content was observed for any of the poloxamer 188 compositions at any temperatures. All residual moisture values fell below the spec of 3% after 6 months.

Upon visual inspection, concentration of sucrose had no effect on poloxamer 188 formulation cake appearance over 6 months at 2-8° C., 30° C., and 40° C. (data not shown).

The results of this study show that, except for aggregation, compositions of efanesoctocog alfa formulated with either 0.5% (w/v) PS80 or 0.1% (w/v) poloxamer 188 have similar stability attributes. Evaluation of other stability attributes such as glass transition temperature, turbidity, and residual moisture did not show any significant differences based on choice of surfactant.

### Example 3: Effect of Arginine Concentration on Stability of Efanesoctocog Alfa Compositions

Efanesoctocog alfa has demonstrated a tendency to undergo reversible self-association following freeze/thaw (F/T) stress. Use of arginine in efanesoctocog alfa compositions may provide a protective effect against this F/T induced molecular self association. To determine the optimal concentration of arginine for efanesoctocog alfa compositions, several compositions comprising various concentrations of arginine were prepared and aggregation levels were tested by SEC. Each tested composition comprised concentrated efanesoctocog alfa (>2 mg/mL), 10 mM L-histidine, 5 mM CaCl₂), 5% (w/v) sucrose, 0.1% (w/v) poloxamer 188 (P188), and L-arginine-HCl at a concentration of 125, 150, 175, 200, or 250 mM.

To prepare the compositions for testing, efanesoctocog alfa compositions were lyophilized using the LyoStar2 development-scale lyophilizer (Protein Pharmaceutical Development Laboratories, Cambridge, MA). To test each sample, compositions were thawed and injected immediately into the HPLC for SEC analysis. Samples were analyzed immediately after thawing, and after 40, 80, 120, and 160 minutes post-thaw.

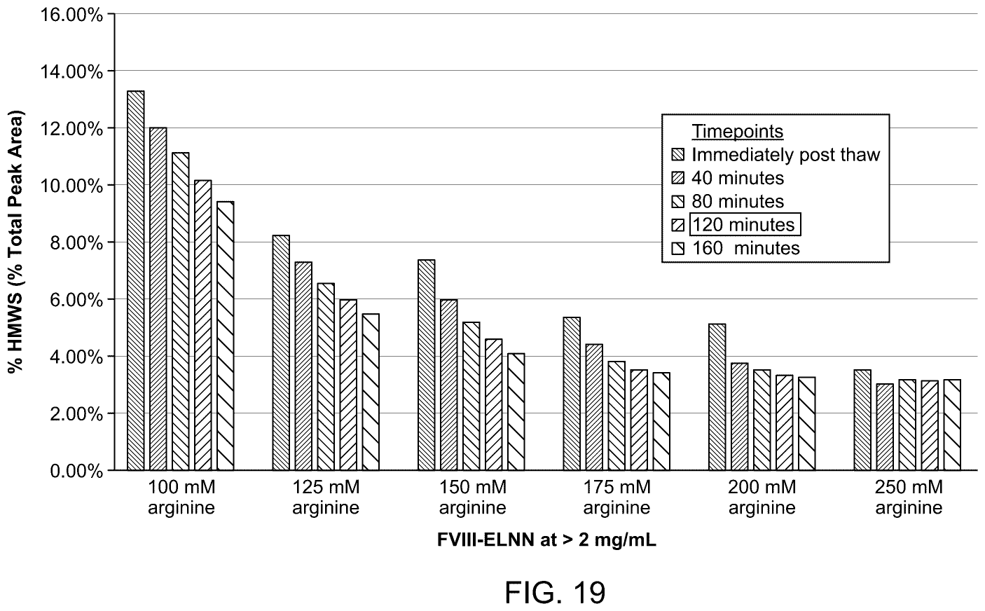

The results of the aggregation assessments (% HMWS) for all samples are shown in FIG. 19. Efanesoctocog alfa compositions comprising arginine HCl levels below 250 mM demonstrate increased aggregation levels immediately upon thawing, followed by a decrease in aggregate levels over time (FIGS. 18A-18C). This aggregation trend suggests formation and disassociation of HMWS that were formed due to F/T induced molecular self association of efanesoctocog alfa. However, efanesoctocog alfa compositions comprising arginine HCl concentrations of 250 mM demonstrate relatively low aggregation levels immediately after thawing, and these levels remain consistent over time (see FIGS. 18A-18C, far right columns).

The results of this study indicate that efanesoctocog alfa compositions with arginine HCl concentrations of 250 mM may be better protected from F/T induced molecular self association. Thus 250 mM appears to be the preferred concentration of arginine HCl for use in formulating efanesoctocog alfa compositions.

### Example 4: Evaluation of Pharmacokinetics of Efanesoctocog Alfa Compositions Comprising Different Surfactants in Non-Human Primates

The objective of this study was to compare the pharmacokinetic (PK) profile of efanesoctocog alfa compositions comprising either polysorbate 80 (PS80) or poloxamer 188 (P188) in cynomolgus monkeys.

The following two vehicles were tested:
Vehicle 1: 10 mM L-Histidine, 250 mM Arginine-HCl, 5 mM Calcium Chloride, 5% w/v Sucrose, 0.05% Polysorbate 80
Vehicle 2: 10 mM L-Histidine, 250 mM Arginine-HCl, 5 mM Calcium Chloride, 5% w/v Sucrose, 0.1% Poloxamer 188

The first group of subjects were administered a single intravenous (IV) bolus injection of 75 IU/kg/dose of efanesoctocog alfa in Vehicle 1. The second group of subjects were administered a single intravenous (IV) bolus injection of 75 IU/kg/dose of efanesoctocog alfa in Vehicle 2. Both compositions included efanesoctocog alfa at a concentration of 37.5 IU/mL. A volume of 2 mL was administered to each subject. Blood samples were collected for PK analysis at Predose, 0.25, 1, 3, 8, 24, 48, 96, 168, 240, and 336 hours.

For the dose formulations, the concentrations of formulation samples analyzed were 88.6% and 92.0% of their respective theoretical concentration, for groups 1 and 2, respectively. The chromogenic activity of the formulation samples analyzed (following repeat analysis) were on a lower range and were 70.5% and 64.9% of their respective theoretical concentration, for groups 1 and 2, respectively.

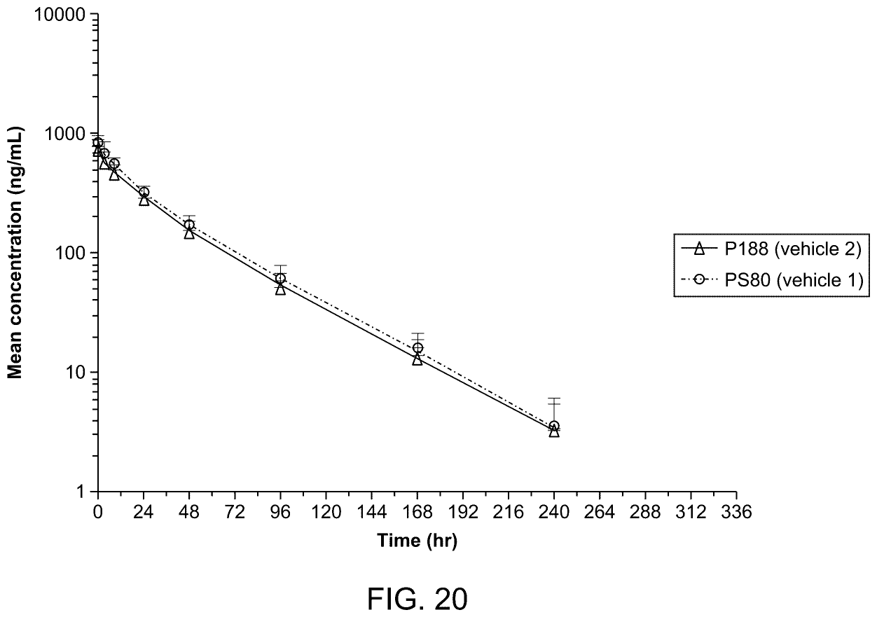

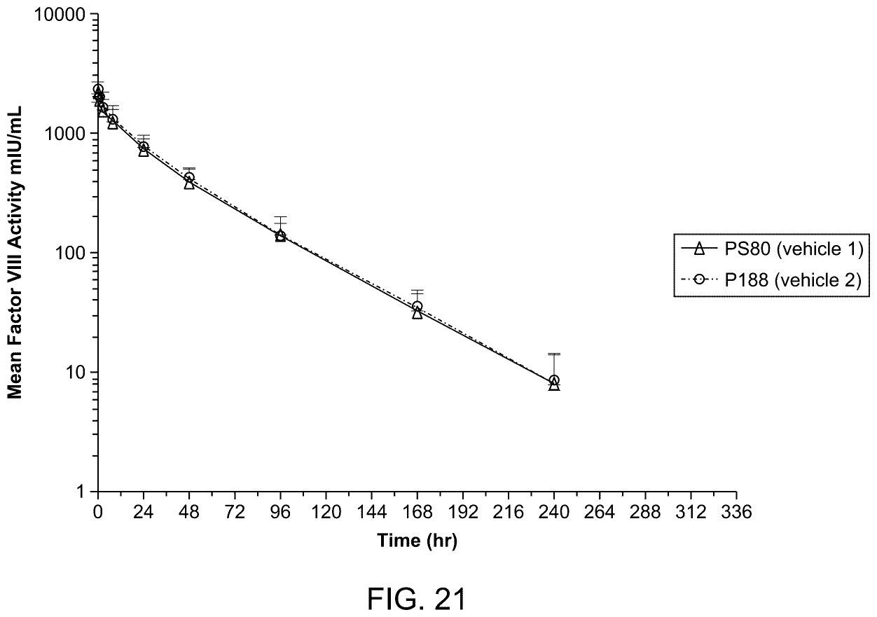

Efanesoctocog alfa protein concentration was determined by ELISA. Efanesoctocog alfa protein concentration over time is shown in FIG. 20. Efanesoctocog alfa protein activity was determined using the chromogenic assay. Efanesoctocog alfa activity over time is shown in FIG. 21. Both protein concentration and activity profiles were comparable for compositions comprising either PS80 or P188.

PK parameters for efanesoctocog alfa protein concentration and activity were determined and are presented in Table 2. Values are presented as mean (+SD) and were generated from 4 animals for each group. As can be seen in Table 2, PK parameters were comparable for compositions comprising either PS80 or P188.

Efanesoctocog alfa protein concentration and activity parameters were directly compared between subjects receiving P188 or PS80 compositions. For protein concentration, the Cₘₐₓ ratio and AUC₍₀₋ₜ₎ ratio were determined to be 91.8% and 89.8%, respectively. For chromogenic activity, the Cₘₐₓ ratio and AUC₍₀₋₁₎ ratio were determined to be 106% and 105%, respectively.

The Tₘₐₓ was observed at first time point after dosing (i.e., 0.25 hour post dose) for efanesoctocog alfa and efanesoctocog alfa activity. The mean Cₘₐₓ was 841 and 772 ng/mL for efanesoctocog alfa concentration and 2110 and 2230 mIU/mL for efanesoctocog alfa activity, with Vehicle 1 and 2, respectively. The mean AUC₍₀₋ₜ₎ was 25400 and 22800 hr*ng/ml for efanesoctocog alfa concentration and 59600 and 62800 hr*mIU/mL for efanesoctocog alfa activity, with Vehicle 1 and 2, respectively. The mean t1/2 was 36.1 and 35.8 hours for efanesoctocog alfa concentration and 34.4 and 35.3 hours for efanesoctocog alfa activity, with vehicle 1 and 2, respectively. The mean CLₚₗₐₛₘₐ was 1.33 and 1.44 mL/hr/kg for efanesoctocog alfa concentration and 1.29 and 1.20 mL/hr/kg for efanesoctocog alfa activity, with vehicle 1 and 2, respectively. The mean Vd was 69.0 and 74.4 mL/kg for efanesoctocog alfa concentration and 63.8 and 61.4 mL/kg for BIVV001 activity, with vehicle 1 and 2, respectively.

In conclusion, the PK parameters of efanesoctocog alfa compositions were relatively similar regardless of selection of vehicle between PS80 and P188. There were no test item-related clinical signs or any injection site irritation noted following administration to subjects. All toxicokinetic parameters for each group were generated from 4 animals. Values are rounded to 3 significant figures (except Tₘₐₓ).

TABLE 2
PK parameters for efanesoctocog alfa compositions in cynomolgus monkeys.

| | | Cₘₐₓ | Tₘₐₓ Median | AUC₍₀₋ₜ₎ | | CLₚₗₐₛₘₐ | Vd |
|---|---|---|---|---|---|---|---|
| Analyte | Vehicle | (ng/mL) | (range) (hr) | (hr*ng/mL) | t_{1/2} (hr) | (mL/hr/kg) | (mL/kg) |
| Efa | PS80 | 841 ± 119 | 0.25 (0.25-0.25) | 25400 ± 4940 | 36.1 ± 3.53 | 1.33 ± 0.300 | 69.0 ± 14.2 |
| concentration | P188 | 772 ± 101 | 0.25 (0.25-0.25) | 22800 ± 2200 | 35.8 ± 5.50 | 1.44 ± 0.133 | 74.4 ± 15.4 |
| | | Cₘₐₓ | Tₘₐₓ Median | AUC₍₀₋ₜ₎ | | CLₚₗₐₛₘₐ | Vd |
| Analyte | Vehicle | (mIU/mL) | (range) (hr) | (hr*mIU/mL) | t_{1/2} (hr) | (mL/hr/kg) | (mL/kg) |
| Efa | PS80 | 2110 ± 382 | 0.25 (0.25-0.25) | 59600 ± 14300 | 34.4 ± 3.05 | 1.29 ± 0.276 | 63.8 ± 11.7 |
| Activity | P188 | 2230 ± 342 | 0.25 (0.25-0.25) | 62800 ± 8640 | 35.3 ± 5.96 | 1.20 ± 0.153 | 61.4 ± 16.2 |

The foregoing description of the specific embodiments will so fully reveal the general nature of the disclosure that others can, by applying knowledge within the skill of the art, readily modify and/or adapt for various applications such specific embodiments, without undue experimentation, without departing from the general concept of the present disclosure. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

Other embodiments of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

All patents and publications cited herein are incorporated by reference herein in their entirety.

TABLE 3
SEQUENCES

Exemplary chimeric protein sequences
A-FVIII(ELNN)-Fc: SEQ ID NO: 1

| | |
|---|---|
| ATRRYYLGAV ELSWDYMQSD LGELPVDARF PPRVPKSFPF NTSVVYKKTL FVEFTDHLFN | 60 |
| IAKPRPPWMG LLGPTIQAEV YDTVVITLKN MASHPVSLHA VGVSYWKASE GAEYDDQTSQ | 120 |
| REKEDDKVFP GGSHTYVWQV LKENGPMASD PLCLTYSYLS HVDLVKDLNS GLIGALLVCR | 180 |
| EGSLAKEKTQ TLHKFILLFA VFDEGKSWHS ETKNSLMQDR DAASARAWPK MHTVNGYVNR | 240 |
| SLPGLIGCHR KSVYWHVIGM GTTPEVHSIF LEGHTFLVRN HRQASLEISP ITFLTAQTLL | 300 |
| MDLGQFLLFC HISSHQHDGM EAYVKVDSCP EEPQLRMKNN EEAEDYDDDL TDSEMDVVRF | 360 |
| DDDNSPSFIQ IRSVAKKHPK TWVHYIAAEE EDWDYAPLVL APDDRSYKSQ YLNNGPQRIG | 420 |
| RKYKKVRFMA YTDETFKTRE AIQHESGILG PLLYGEVGDT LLIIFKNQAS RPYNIYPHGI | 480 |
| TDVRPLYSRR LPKGVKHLKD FPILPGEIFK YKWTVTVEDG PTKSDPRCLT RYYSSFVNME | 540 |
| RDLASGLIGP LLICYKESVD QRGNQIMSDK RNVILFSVFD ENRSWYLTEN IQRFLPNPAG | 600 |
| VQLEDPEFQA SNIMHSINGY VFDSLQLSVC LHEVAYWYIL SIGAQTDFLS VFFSGYTFKH | 660 |
| KMVYEDTLTL FPFSGETVFM SMENPGLWIL GCHNSDFRNR GMTALLKVSS CDKNTGDYYE | 720 |
| DSYEDISAYL LSKNNAIEPR SFSQNGTSES ATPESGPGSE PATSGSETPG TSESATPESG | 780 |
| PGSEPATSGS ETPGTSESAT PESGPGTSTE PSEGSAPGSP AGSPTSTEEG TSESATPESG | 840 |
| PGSEPATSGS ETPGTSESAT PESGPGSPAG SPTSTEEGSP AGSPTSTEEG TSTEPSEGSA | 900 |
| PGTSESATPE SGPGTSESAT PESGPGTSES ATPESGPGSE PATSGSETPG SEPATSGSET | 960 |
| PGSPAGSPTS TEEGTSTEPS EGSAPGTSTE PSEGSAPGSE PATSGSETPG TSESATPESG | 1020 |
| PGTSTEPSEG SAPASSEITR TTLQSDQEEI DYDDTISVEM KKEDFDIYDE DENQSPRSFQ | 1080 |
| KKTRHYFIAA VERLWDYGMS SSPHVLRNRA QSGSVPQFKK VVFQEFTDGS FTQPLYRGEL | 1140 |
| NEHLGLLGPY IRAEVEDNIM VTFRNQASRP YSFYSSLISY EEDQRQGAEP RKNFVKPNET | 1200 |
| KTYFWKVQHH MAPTKDEFDC KAWAYFSDVD LEKDVHSGLI GPLLVCHTNT LNPAHGRQVT | 1260 |
| VQEFALFFTI FDETKSWYFT ENMERNCRAP CNIQMEDPTF KENYRFHAIN GYIMDTLPGL | 1320 |
| VMAQDQRIRW YLLSMGSNEN IHSIHFSGHV FTVRKKEEYK MALYNLYPGV FETVEMLPSK | 1380 |
| AGIWRVECLI GEHLHAGMST LFLVYSNKCQ TPLGMASGHI RDFQITASGQ YGQWAPKLAR | 1440 |
| LHYSGSINAW STKEPFSWIK VDLLAPMIIH GIKTQGARQK FSSLYISQFI IMYSLDGKKW | 1500 |
| QTYRGNSTGT LMVFFGNVDS SGIKHNIFNP PIIARYIRLH PTHYSIRSTL RMELMGCDLN | 1560 |
| SCSMPLGMES KAISDAQITA SSYFTNMFAT WSPSKARLHL QGRSNAWRPQ VNNPKEWLQV | 1620 |
| DFQKTMKVTG VTTQGVKSLL TSMYVKEFLI SSSQDGHQWT LFFQNGKVKV FQGNQDSFTP | 1680 |
| VVNSLDPPLL TRYLRIHPQS WVHQIALRME VLGCEAQDLY DKTHTCPPCP APELLGGPSV | 1740 |
| FLFPPKPKDT LMISRTPEVT CVVVDVSHED PEVKFNWYVD GVEVHNAKTK PREEQYNSTY | 1800 |
| RVVSVLTVLH QDWLNGKEYK CKVSNKALPA PIEKTISKAK GQPREPQVYT LPPSRDELTK | 1860 |
| NQVSLTCLVK GFYPSDIAVE WESNGQPENN YKTTPPVLDS DGSFFLYSKL TVDKSRWQQG | 1920 |
| NVFSCSVMHE ALHNHYTQKS LSLSPG | |
| VWF(D'D3)-ELNN-a2 Linker-Fc: SEQ ID NO: 2 | |
| SLSCRPPMVK LVCPADNLRA EGLECTKTCQ NYDLECMSMG CVSGCLCPPG MVRHENRCVA | 60 |
| LERCPCFHQG KEYAPGETVK IGCNTCVCRD RKWNCTDHVC DATCSTIGMA HYLTFDGLKY | 120 |
| LFPGECQYVL VQDYCGSNPG TFRILVGNKG CSHPSVKCKK RVTILVEGGE IELFDGEVNV | 180 |
| KRPMKDETHF EVVESGRYII LLLGKALSVV WDRHLSISVV LKQTYQEKVC GLCGNFDGIQ | 240 |
| NNDLTSSNLQ VEEDPVDFGN SWKVSSQCAD TRKVPLDSSP ATCHNNIMKQ TMVDSSCRIL | 300 |
| TSDVFQDCNK LVDPEPYLDV CIYDTCSCES IGDCAAFCDT IAAYAHVCAQ HGKVVTWRTA | 360 |
| TLCPQSCEER NLRENGYEAE WRYNSCAPAC QVTCQHPEPL ACPVQCVEGC HAHCPPGKIL | 420 |
| DELLQTCVDP EDCPVCEVAG RRFASGKKVT LNPSDPEHCQ ICHCDVVNLT CEACQEPGTS | 480 |
| ESATPESGPG SEPATSGSET PGTSESATPE SGPGSEPATS GSETPGTSES ATPESGPGTS | 540 |
| TEPSEGSAPG SPAGSPTSTE EGTSESATPE SGPGSEPATS GSETPGTSES ATPESGPGSP | 600 |
| AGSPTSTEEG SPAGSPTSTE EGASSDKNTG DYYEDSYEDI SAYLLSKNNA IEPRSFSDKT | 660 |
| HTCPPCPAPE LLGGPSVFLF PPKPKDTLMI SRTPEVTCVV VDVSHEDPEV KFNWYVDGVE | 720 |
| VHNAKTKPRE EQYNSTYRVV SVLTVLHQDW LNGKEYKCKV SNKALPAPIE KTISKAKGQP | 780 |
| REPQVYTLPP SRDELTKNQV SLTCLVKGFY PSDIAVEWES NGQPENNYKT TPPVLDSDGS | 840 |
| FFLYSKLTVD KSRWQQGNVF SCSVMHEALH NHYTQKSLSL SPG | 883 |

Amino acid sequence of FVIII protein (312A)-includes signal peptide: SEQ
ID NO: 3

| | |
|---|---|
| 1 | MQIELSTCFF LCLLRFCESA TRRYYLGAVE LSWDYMQSDL GELPVDARFP |
| 51 | PRVPKSFPEN TSVVYKKTLF VEFTDHLENI AKPRPPWMGL LGPTIQAEVY |
| 101 | DTVVITLKNM ASHPVSLHAV GVSYWKASEG AEYDDQTSQR EKEDDKVFPG |
| 151 | GSHTYVWQVL KENGPMASDP LCLTYSYLSH VDLVKDLNSG LIGALLVCRE |
| 201 | GSLAKEKTQT LHKFILLFAV FDEGKSWHSE TKNSLMQDRD AASARAWPKM |
| 251 | HTVNGYVNRS LPGLIGCHRK SVYWHVIGMG TTPEVHSIFL EGHTFLVRNH |
| 301 | RQASLEISPI TFLTAQTLLM DLGQFLLFCH ISSHQHDGME AYVKVDSCPE |
| 351 | EPQLRMKNNE EAEDYDDDLT DSEMDVVRFD DDNSPSFIQI RSVAKKHPKT |
| 401 | WVHYIAAEEE DWDYAPLVLA PDDRSYKSQY LNNGPQRIGR KYKKVRFMAY |
| 451 | TDETEKTREA IQHESGILGP LLYGEVGDTL LIIFKNQASR PYNIYPHGIT |
| 501 | DVRPLYSRRL PKGVKHLKDF PILPGEIFKY KWTVTVEDGP TKSDPRCLTR |
| 551 | YYSSFVNMER DLASGLIGPL LICYKESVDQ RGNQIMSDKR NVILFSVFDE |
| 601 | NRSWYLTENI QRFLPNPAGV QLEDPEFQAS NIMHSINGYV FDSLQLSVCL |
| 651 | HEVAYWYILS IGAQTDFLSV FFSGYTFKHK MVYEDTLTLF PFSGETVFMS |
| 701 | MENPGLWILG CHNSDFRNRG MTALLKVSSC DKNTGDYYED SYEDISAYLL |
| 751 | SKNNAIEPRS FSQNGTSESA TPESGPGSEP ATSGSETPGT SESATPESGP |
| 801 | GSEPATSGSE TPGTSESATP ESGPGTSTEP SEGSAPGSPA GSPTSTEEGT |
| 851 | SESATPESGP GSEPATSGSE TPGTSESATP ESGPGSPAGS PTSTEEGSPA |
| 901 | GSPTSTEEGT STEPSEGSAP GTSESATPES GPGTSESATP ESGPGTSESA |
| 951 | TPESGPGSEP ATSGSETPGS EPATSGSETP GSPAGSPTST EEGTSTEPSE |
| 1001 | GSAPGTSTEP SEGSAPGSEP ATSGSETPGT SESATPESGP GTSTEPSEGS |
| 1051 | APASSEITRT TLQSDQEEID YDDTISVEMK KEDFDIYDED ENQSPRSFQK |
| 1101 | KTRHYFIAAV ERLWDYGMSS SPHVLRNRAQ SGSVPQFKKV VFQEFTDGSF |
| 1151 | TQPLYRGELN EHLGLLGPYI RAEVEDNIMV TFRNQASRPY SFYSSLISYE |
| 1201 | EDQRQGAEPR KNFVKPNETK TYFWKVQHHM APTKDEFDCK AWAYFSDVDL |
| 1251 | EKDVHSGLIG PLLVCHTNTL NPAHGRQVTV QEFALFFTIF DETKSWYFTE |
| 1301 | NMERNCRAPC NIQMEDPTFK ENYRFHAING YIMDTLPGLV MAQDQRIRWY |
| 1351 | LLSMGSNENI HSIHFSGHVF TVRKKEEYKM ALYNLYPGVF ETVEMLPSKA |
| 1401 | GIWRVECLIG EHLHAGMSTL FLVYSNKCQT PLGMASGHIR DFQITASGQY |
| 1451 | GQWAPKLARL HYSGSINAWS TKEPFSWIKV DLLAPMIIHG IKTQGARQKF |
| 1501 | SSLYISQFII MYSLDGKKWQ TYRGNSTGTL MVFFGNVDSS GIKHNIFNPP |
| 1551 | IIARYIRLHP THYSIRSTLR MELMGCDLNS CSMPLGMESK AISDAQITAS |
| 1601 | SYFTNMFATW SPSKARLHLQ GRSNAWRPQV NNPKEWLQVD FQKTMKVTGV |
| 1651 | TTQGVKSLLT SMYVKEFLIS SSQDGHQWTL FFQNGKVKVF QGNQDSFTPV |
| 1701 | VNSLDPPLLT RYLRIHPQSW VHQIALRMEV LGCEAQDLYD KTHTCPPCPA |
| 1751 | PELLGGPSVF LFPPKPKDTL MISRTPEVTC VVVDVSHEDP EVKFNWYVDG |
| 1801 | VEVHNAKTKP REEQYNSTYR VVSVLTVLHQ DWLNGKEYKC KVSNKALPAP |
| 1851 | IEKTISKAKG QPREPQVYTL PPSRDELTKN QVSLTCLVKG FYPSDIAVEW |
| 1901 | ESNGQPENNY KTTPPVLDSD GSFFLYSKLT VDKSRWQQGN VFSCSVMHEA |
| 1951 | LHNHYTQKSL SLSPG* |

Nucleotide sequence encoding FVIII 312A: SEQ ID NO: 4

| | |
|---|---|
| 1 | ATGCAAATAG AGCTCTCCAC CTGCTTCTTT CTGTGCCTTT TGCGATTCTG |
| 51 | CTTTAGTGCC ACCAGAAGAT ACTACCTGGG TGCAGTGGAA CTGTCATGGG |
| 101 | ACTATATGCA AAGTGATCTC GGTGAGCTGC CTGTGGACGC AAGATTTCCT |
| 151 | CCTAGAGTGC CAAAATCTTT TCCATTCAAC ACCTCAGTCG TGTACAAAAA |
| 201 | GACTCTGTTT GTAGAATTCA CGGATCACCT TTTCAACATC GCTAAGCCAA |
| 251 | GGCCACCCTG GATGGGTCTG CTAGGTCCTA CCATCCAGGC TGAGGTTTAT |
| 301 | GATACAGTGG TCATTACACT TAAGAACATG GCTTCCCATC CTGTCAGTCT |
| 351 | TCATGCTGTT GGTGTATCCT ACTGGAAAGC TTCTGAGGGA GCTGAATATG |
| 401 | ATGATCAGAC CAGTCAAAGG GAGAAAGAAG ATGATAAAGT CTTCCCTGGT |
| 451 | GGAAGCCATA CATATGTCTG GCAGGTCCTG AAAGAGAATG GTCCAATGGC |
| 501 | CTCTGACCCA CTGTGCCTTA CCTACTCATA TCTTTCTCAT GTGGACCTGG |
| 551 | TAAAAGACTT GAATTCAGGC CTCATTGGAG CCCTACTAGT ATGTAGAGAA |
| 601 | GGGAGTCTGG CCAAGGAAAA GACACAGACC TTGCACAAAT TTATACTACT |
| 651 | TTTTGCTGTA TTTGATGAAG GGAAAAGTTG GCACTCAGAA ACAAAGAACT |
| 701 | CCTTGATGCA GGATAGGGAT GCTGCATCTG CTCGGGCCTG GCCTAAAATG |
| 751 | CACACAGTCA ATGGTTATGT AAACAGGTCT CTGCCAGGTC TGATTGGATG |
| 801 | CCACAGGAAA TCAGTCTATT GGCATGTGAT TGGAATGGGC ACCACTCCTG |
| 851 | AAGTGCACTC AATATTCCTC GAAGGTCACA CATTTCTTGT GAGGAACCAT |
| 901 | CGCCAGGCGT CCTTGGAAAT CTCGCCAATA ACTTTCCTTA CTGCTCAAAC |
| 951 | ACTCTTGATG GACCTTGGAC AGTTTCTACT GTTTTGTCAT ATCTCTTCCC |
| 1001 | ACCAACATGA TGGCATGGAA GCTTATGTCA AAGTAGACAG CTGTCCAGAG |
| 1051 | GAACCCCAAC TACGAATGAA AAATAATGAA GAAGCGGAAG ACTATGATGA |
| 1101 | TGATCTTACT GATTCTGAAA TGGATGTGGT CAGGTTTGAT GATGACAACT |
| 1151 | CTCCTTCCTT TATCCAAATT CGCTCAGTTG CCAAGAAGCA TCCTAAAACT |
| 1201 | TGGGTACATT ACATTGCTGC TGAAGAGGAG GACTGGGACT ATGCTCCCTT |
| 1251 | AGTCCTCGCC CCCGATGACA GAAGTTATAA AAGTCAATAT TTGAACAATG |
| 1301 | GCCCTCAGCG GATTGGTAGG AAGTACAAAA AAGTCCGATT TATGGCATAC |
| 1351 | ACAGATGAAA CCTTTAAGAC TCGTGAAGCT ATTCAGCATG AATCAGGAAT |
| 1401 | CTTGGGACCT TTACTTTATG GGGAAGTTGG AGACACACTG TTGATTATAT |
| 1451 | TTAAGAATCA AGCAAGCAGA CCATATAACA TCTACCCTCA CGGAATCACT |
| 1501 | GATGTCCGTC CTTTGTATTC AAGGAGATTA CCAAAAGGTG TAAAACATTT |
| 1551 | GAAGGATTTT CCAATTCTGC CAGGAGAAAT ATTCAAATAT AAATGGACAG |
| 1601 | TGACTGTAGA AGATGGGCCA ACTAAATCAG ATCCTCGGTG CCTGACCCGC |
| 1651 | TATTACTCTA GTTTCGTTAA TATGGAGAGA GATCTAGCTT CAGGACTCAT |
| 1701 | TGGCCCTCTC CTCATCTGCT ACAAAGAATC TGTAGATCAA AGAGGAAACC |
| 1751 | AGATAATGTC AGACAAGAGG AATGTCATCC TGTTTTCTGT ATTTGATGAG |
| 1801 | AACCGAAGCT GGTACCTCAC AGAGAATATA CAACGCTTTC TCCCCAATCC |
| 1851 | AGCTGGAGTG CAGCTTGAGG ATCCAGAGTT CCAAGCCTCC AACATCATGC |
| 1901 | ACAGCATCAA TGGCTATGTT TTTGATAGTT TGCAGTTGTC AGTTTGTTTG |
| 1951 | CATGAGGTGG CATACTGGTA CATTCTAAGC ATTGGAGCAC AGACTGACTT |
| 2001 | CCTTTCTGTC TTCTTCTCTG GATATACCTT CAAACACAAA ATGGTCTATG |
| 2051 | AAGACACACT CACCCTATTC CCATTCTCAG GAGAAACTGT CTTCATGTCG |
| 2101 | ATGGAAAACC CAGGTCTATG GATTCTGGGG TGCCACAACT CAGACTTTCG |
| 2151 | GAACAGAGGC ATGACCGCCT TACTGAAGGT TTCTAGTTGT GACAAGAACA |
| 2201 | CTGGTGATTA TTACGAGGAC AGTTATGAAG ATATTTCAGC ATACTTGCTG |
| 2251 | AGTAAAAACA ATGCCATTGA ACCAAGAAGC TTCTCTCAAA ACGGTACCTC |
| 2301 | AGAGTCTGCT ACCCCCGAGT CAGGGCCAGG ATCAGAGCCA GCCACCTCCG |
| 2351 | GGTCTGAGAC ACCCGGGACT TCCGAGAGTG CCACCCCTGA GTCCGGACCC |
| 2401 | GGGTCCGAGC CCGCCACTTC CGGCTCCGAA ACTCCCGGCA CAAGCGAGAG |
| 2451 | CGCTACCCCA GAGTCAGGAC CAGGAACATC TACAGAGCCC TCTGAAGGCT |
| 2501 | CCGCTCCAGG GTCCCCAGCC GGCAGTCCCA CTAGCACCGA GGAGGGAACC |
| 2551 | TCTGAAAGCG CCACACCCGA ATCAGGGCCA GGGTCTGAGC CTGCTACCAG |
| 2601 | CGGCAGCGAG ACACCAGGCA CCTCTGAGTC CGCCACACCA GAGTCCGGAC |
| 2651 | CCGGATCTCC CGCTGGGAGC CCCACCTCCA CTGAGGAGGG ATCTCCTGCT |
| 2701 | GGCTCTCCAA CATCTACTGA GGAAGGAACC TCAACCGAGC CATCCGAGGG |
| 2751 | ATCAGCTCCC GGCACCTCAG AGTCGGCAAC CCCGGAGTCT GGACCCGGAA |
| 2801 | CTTCCGAAAG TGCCACACCA GAGTCCGGTC CCGGGACTTC AGAATCAGCA |
| 2851 | ACACCCGAGT CCGGCCCTGG GTCTGAACCC GCCACAAGTG GTAGTGAGAC |
| 2901 | ACCAGGATCA GAACCTGCTA CCTCAGGGTC AGAGACACCC GGATCTCCGG |
| 2951 | CAGGCTCACC AACCTCCACT GAGGAGGGCA CCAGCACAGA ACCAAGCGAG |
| 3001 | GGCTCCGCAC CCGGAACAAG CACTGAACCC AGTGAGGGTT CAGCACCCGG |
| 3051 | CTCTGAGCCG GCCACAAGTG GCAGTGAGAC ACCCGGCACT TCAGAGAGTG |
| 3101 | CCACCCCCGA GAGTGGCCCA GGCACTAGTA CCGAGCCCTC TGAAGGCAGT |
| 3151 | GCGCCAGCCT CGAGCGAAAT AACTCGTACT ACTCTTCAGT CAGATCAAGA |
| 3201 | GGAAATTGAC TATGATGATA CCATATCAGT TGAAATGAAG AAGGAAGATT |
| 3251 | TTGACATTTA TGATGAGGAT GAAAATCAGA GCCCCCGCAG CTTTCAAAAG |
| 3301 | AAAACACGAC ACTATTTTAT TGCTGCAGTG GAGAGGCTCT GGGATTATGG |
| 3351 | GATGAGTAGC TCCCCACATG TTCTAAGAAA CAGGGCTCAG AGTGGCAGTG |
| 3401 | TCCCTCAGTT CAAGAAAGTT GTTTTCCAGG AATTTACTGA TGGCTCCTTT |
| 3451 | ACTCAGCCCT TATACCGTGG AGAACTAAAT GAACATTTGG GACTCCTGGG |
| 3501 | GCCATATATA AGAGCAGAAG TTGAAGATAA TATCATGGTA ACTTTCAGAA |
| 3551 | ATCAGGCCTC TCGTCCCTAT TCCTTCTATT CTAGCCTTAT TTCTTATGAG |
| 3601 | GAAGATCAGA GGCAAGGAGC AGAACCTAGA AAAAACTTTG TCAAGCCTAA |
| 3651 | TGAAACCAAA ACTTACTTTT GGAAAGTGCA ACATCATATG GCACCCACTA |
| 3701 | AAGATGAGTT TGACTGCAAA GCCTGGGCTT ATTTCTCTGA TGTTGACCTG |
| 3751 | GAAAAAGATG TGCACTCAGG CCTGATTGGA CCCCTTCTGG TCTGCCACAC |
| 3801 | TAACACACTG AACCCTGCTC ATGGGAGACA AGTGACAGTA CAGGAATTTG |
| 3851 | CTCTGTTTTT CACCATCTTT GATGAGACCA AAAGCTGGTA CTTCACTGAA |
| 3901 | AATATGGAAA GAAACTGCAG GGCTCCCTGC AATATCCAGA TGGAAGATCC |
| 3951 | CACTTTTAAA GAGAATTATC GCTTCCATGC AATCAATGGC TACATAATGG |
| 4001 | ATACACTACC TGGCTTAGTA ATGGCTCAGG ATCAAAGGAT TCGATGGTAT |
| 4051 | CTGCTCAGCA TGGGCAGCAA TGAAAACATC CATTCTATTC ATTTCAGTGG |
| 4101 | ACATGTGTTC ACTGTACGAA AAAAAGAGGA GTATAAAATG GCACTGTACA |
| 4151 | ATCTCTATCC AGGTGTTTTT GAGACAGTGG AAATGTTACC ATCCAAAGCT |
| 4201 | GGAATTTGGC GGGTGGAATG CCTTATTGGC GAGCATCTAC ATGCTGGGAT |
| 4251 | GAGCACACTT TTTCTGGTGT ACAGCAATAA GTGTCAGACT CCCCTGGGAA |
| 4301 | TGGCTTCTGG ACACATTAGA GATTTTCAGA TTACAGCTTC AGGACAATAT |
| 4351 | GGACAGTGGG CCCCAAAGCT GGCCAGACTT CATTATTCCG GATCAATCAA |
| 4401 | TGCCTGGAGC ACCAAGGAGC CCTTTTCTTG GATCAAGGTG GATCTGTTGG |
| 4451 | CACCAATGAT TATTCACGGC ATCAAGACCC AGGGTGCCCG TCAGAAGTTC |
| 4501 | TCCAGCCTCT ACATCTCTCA GTTTATCATC ATGTATAGTC TTGATGGGAA |
| 4551 | GAAGTGGCAG ACTTATCGAG GAAATTCCAC TGGAACCTTA ATGGTCTTCT |
| 4601 | TTGGCAATGT GGATTCATCT GGGATAAAAC ACAATATTTT TAACCCTCCA |
| 4651 | ATTATTGCTC GATACATCCG TTTGCACCCA ACTCATTATA GCATTCGCAG |
| 4701 | CACTCTTCGC ATGGAGTTGA TGGGCTGTGA TTTAAATAGT TGCAGCATGC |
| 4751 | CATTGGGAAT GGAGAGTAAA GCAATATCAG ATGCACAGAT TACTGCTTCA |
| 4801 | TCCTACTTTA CCAATATGTT TGCCACCTGG TCTCCTTCAA AAGCTCGACT |
| 4851 | TCACCTCCAA GGGAGGAGTA ATGCCTGGAG ACCTCAGGTG AATAATCCAA |
| 4901 | AAGAGTGGCT GCAAGTGGAC TTCCAGAAGA CAATGAAAGT CACAGGAGTA |
| 4951 | ACTACTCAGG GAGTAAAATC TCTGCTTACC AGCATGTATG TGAAGGAGTT |
| 5001 | CCTCATCTCC AGCAGTCAAG ATGGCCATCA GTGGACTCTC TTTTTTCAGA |
| 5051 | ATGGCAAAGT AAAGGTTTTT CAGGGAAATC AAGACTCCTT CACACCTGTG |
| 5101 | GTGAACTCTC TAGACCCACC GTTACTGACT CGCTACCTTC GAATTCACCC |
| 5151 | CCAGAGTTGG GTGCACCAGA TTGCCCTGAG GATGGAGGTT CTGGGCTGCG |
| 5201 | AGGCACAGGA CCTCTACGAC AAAACTCACA CATGCCCACC GTGCCCAGCT |
| 5251 | CCAGAACTCC TGGGCGGACC GTCAGTCTTC CTCTTCCCCC CAAAACCCAA |
| 5301 | GGACACCCTC ATGATCTCCC GGACCCCTGA GGTCACATGC GTGGTGGTGG |
| 5351 | ACGTGAGCCA CGAAGACCCT GAGGTCAAGT TCAACTGGTA TGTGGACGGC |
| 5401 | GTGGAAGTGC ATAATGCCAA GACAAAGCCG CGGGAGGAGC AGTACAACAG |
| 5451 | CACGTACCGT GTGGTCAGCG TCCTCACCGT CCTGCACCAA GACTGGCTGA |
| 5501 | ATGGCAAGGA GTACAAGTGC AAGGTCTCCA ACAAAGCCCT CCCAGCCCCC |
| 5551 | ATCGAGAAAA CCATCTCCAA AGCCAAAGGG CAGCCCCGAG AACCACAGGT |
| 5601 | GTACACCCTG CCCCCATCCC GGGATGAGCT GACCAAGAAC CAAGTTAGCC |
| 5651 | TGACCTGCCT GGTCAAAGGC TTCTATCCCA GCGACATCGC CGTGGAGTGG |
| 5701 | GAGAGCAATG GGCAGCCGGA GAACAACTAC AAGACCACGC CTCCCGTGTT |
| 5751 | GGACTCCGAC GGCTCCTTCT TCCTCTACTC CAAGCTCACC GTGGACAAGA |
| 5801 | GCAGGTGGCA GCAGGGGAAC GTCTTCTCAT GCTCCGTGAT GCATGAGGCT |
| 5851 | CTGCACAACC ACTACACGCA GAAGAGCCTC TCCCTGTCTC CGGGTTGA |

Amino acid sequence of VWF059A protein-includes signal peptide and D1D2
region: SEQ ID NO: 5

| | |
|---|---|
| 1 | MIPARFAGVL LALALILPGT LCAEGTRGRS STARCSLFGS DFVNTFDGSM |
| 51 | YSFAGYCSYL LAGGCQKRSF SIIGDFQNGK RVSLSVYLGE FFDIHLFVNG |
| 101 | TVTQGDQRVS MPYASKGLYL ETEAGYYKLS GEAYGFVARI DGSGNFQVLL |
| 151 | SDRYFNKTCG LCGNFNIFAE DDFMTQEGTL TSDPYDFANS WALSSGEQWC |
| 201 | ERASPPSSSC NISSGEMQKG LWEQCQLLKS TSVFARCHPL VDPEPFVALC |
| 251 | EKTLCECAGG LECACPALLE YARTCAQEGM VLYGWTDHSA CSPVCPAGME |
| 301 | YRQCVSPCAR TCQSLHINEM CQERCVDGCS CPEGQLLDEG LCVESTECPC |
| 351 | VHSGKRYPPG TSLSRDCNTC ICRNSQWICS NEECPGECLV TGQSHFKSFD |
| 401 | NRYFTFSGIC QYLLARDCQD HSFSIVIETV QCADDRDAVC TRSVTVRLPG |
| 451 | LHNSLVKLKH GAGVAMDGQD IQLPLLKGDL RIQHTVTASV RLSYGEDLQM |
| 501 | DWDGRGRLLV KLSPVYAGKT CGLCGNYNGN QGDDFLTPSG LAEPRVEDFG |
| 551 | NAWKLHGDCQ DLQKQHSDPC ALNPRMTRFS EEACAVLTSP TFEACHRAVS |
| 601 | PLPYLRNCRY DVCSCSDGRE CLCGALASYA AACAGRGVRV AWREPGRCEL |
| 651 | NCPKGQVYLQ CGTPCNLTCR SLSYPDEECN EACLEGCFCP PGLYMDERGD |
| 701 | CVPKAQCPCY YDGEIFQPED IFSDHHTMCY CEDGFMHCTM SGVPGSLLPD |
| 751 | AVLSSPLSHR SKRSLSCRPP MVKLVCPADN LRAEGLECTK TCQNYDLECM |
| 801 | SMGCVSGCLC PPGMVRHENR CVALERCPCF HQGKEYAPGE TVKIGCNTCV |
| 851 | CRDRKWNCTD HVCDATCSTI GMAHYLTFDG LKYLFPGECQ YVLVQDYCGS |
| 901 | NPGTFRILVG NKGCSHPSVK CKKRVTILVE GGEIELFDGE VNVKRPMKDE |
| 951 | THFEVVESGR YIILLLGKAL SVVWDRHLSI SVVLKQTYQE KVCGLCGNFD |
| 1001 | GIQNNDLTSS NLQVEEDPVD FGNSWKVSSQ CADTRKVPLD SSPATCHNNI |
| 1051 | MKQTMVDSSC RILTSDVFQD CNKLVDPEPY LDVCIYDTCS CESIGDCAAF |
| 1101 | CDTIAAYAHV CAQHGKVVTW RTATLCPQSC EERNLRENGY EAEWRYNSCA |
| 1151 | PACQVTCQHP EPLACPVQCV EGCHAHCPPG KILDELLQTC VDPEDCPVCE |
| 1201 | VAGRRFASGK KVTLNPSDPE HCQICHCDVV NLTCEACQEP GTSESATPES |
| 1251 | GPGSEPATSG SETPGTSESA TPESGPGSEP ATSGSETPGT SESATPESGP |
| 1301 | GTSTEPSEGS APGSPAGSPT STEEGTSESA TPESGPGSEP ATSGSETPGT |
| 1351 | SESATPESGP GSPAGSPTST EEGSPAGSPT STEEGASSDK NTGDYYEDSY |
| 1401 | EDISAYLLSK NNAIEPRSES DKTHTCPPCP APELLGGPSV FLFPPKPKDT |
| 1451 | LMISRTPEVT CVVVDVSHED PEVKFNWYVD GVEVHNAKTK PREEQYNSTY |
| 1501 | RVVSVLTVLH QDWLNGKEYK CKVSNKALPA PIEKTISKAK GQPREPQVYT |
| 1551 | LPPSRDELTK NQVSLTCLVK GFYPSDIAVE WESNGQPENN YKTTPPVLDS |
| 1601 | DGSFFLYSKL TVDKSRWQQG NVFSCSVMHE ALHNHYTQKS LSLSPG* |

Nucleotide sequence encoding VWF059A protein: SEQ ID NO: 6

| | |
|---|---|
| 1 | ATGATTCCTG CCAGATTTGC CGGGGTGCTG CTTGCTCTGG CCCTCATTTT |
| 51 | GCCAGGGACC CTTTGTGCAG AAGGAACTCG CGGCAGGTCA TCCACGGCCC |
| 101 | GATGCAGCCT TTTCGGAAGT GACTTCGTCA ACACCTTTGA TGGGAGCATG |
| 151 | TACAGCTTTG CGGGATACTG CAGTTACCTC CTGGCAGGGG GCTGCCAGAA |
| 201 | ACGCTCCTTC TCGATTATTG GGGACTTCCA GAATGGCAAG AGAGTGAGCC |
| 251 | TCTCCGTGTA TCTTGGGGAA TTTTTTGACA TCCATTTGTT TGTCAATGGT |
| 301 | ACCGTGACAC AGGGGGACCA AAGAGTCTCC ATGCCCTATG CCTCCAAAGG |
| 351 | GCTGTATCTA GAAACTGAGG CTGGGTACTA CAAGCTGTCC GGTGAGGCCT |
| 401 | ATGGCTTTGT GGCCAGGATC GATGGCAGCG GCAACTTTCA AGTCCTGCTG |
| 451 | TCAGACAGAT ACTTCAACAA GACCTGCGGG CTGTGTGGCA ACTTTAACAT |
| 501 | CTTTGCTGAA GATGACTTTA TGACCCAAGA AGGGACCTTG ACCTCGGACC |
| 551 | CTTATGACTT TGCCAACTCA TGGGCTCTGA GCAGTGGAGA ACAGTGGTGT |
| 601 | GAACGGGCAT CTCCTCCCAG CAGCTCATGC AACATCTCCT CTGGGGAAAT |
| 651 | GCAGAAGGGC CTGTGGGAGC AGTGCCAGCT TCTGAAGAGC ACCTCGGTGT |
| 701 | TTGCCCGCTG CCACCCTCTG GTGGACCCCG AGCCTTTTGT GGCCCTGTGT |
| 751 | GAGAAGACTT TGTGTGAGTG TGCTGGGGGG CTGGAGTGCG CCTGCCCTGC |
| 801 | CCTCCTGGAG TACGCCCGGA CCTGTGCCCA GGAGGGAATG GTGCTGTACG |
| 851 | GCTGGACCGA CCACAGCGCG TGCAGCCCAG TGTGCCCTGC TGGTATGGAG |
| 901 | TATAGGCAGT GTGTGTCCCC TTGCGCCAGG ACCTGCCAGA GCCTGCACAT |
| 951 | CAATGAAATG TGTCAGGAGC GATGCGTGGA TGGCTGCAGC TGCCCTGAGG |
| 1001 | GACAGCTCCT GGATGAAGGC CTCTGCGTGG AGAGCACCGA GTGTCCCTGC |
| 1051 | GTGCATTCCG GAAAGCGCTA CCCTCCCGGC ACCTCCCTCT CTCGAGACTG |
| 1101 | CAACACCTGC ATTTGCCGAA ACAGCCAGTG GATCTGCAGC AATGAAGAAT |
| 1151 | GTCCAGGGGA GTGCCTTGTC ACTGGTCAAT CCCACTTCAA GAGCTTTGAC |
| 1201 | AACAGATACT TCACCTTCAG TGGGATCTGC CAGTACCTGC TGGCCCGGGA |
| 1251 | TTGCCAGGAC CACTCCTTCT CCATTGTCAT TGAGACTGTC CAGTGTGCTG |
| 1301 | ATGACCGCGA CGCTGTGTGC ACCCGCTCCG TCACCGTCCG GCTGCCTGGC |
| 1351 | CTGCACAACA GCCTTGTGAA ACTGAAGCAT GGGGCAGGAG TTGCCATGGA |
| 1401 | TGGCCAGGAC ATCCAGCTCC CCCTCCTGAA AGGTGACCTC CGCATCCAGC |
| 1451 | ATACAGTGAC GGCCTCCGTG CGCCTCAGCT ACGGGGAGGA CCTGCAGATG |
| 1501 | GACTGGGATG GCCGCGGGAG GCTGCTGGTG AAGCTGTCCC CCGTCTATGC |
| 1551 | CGGGAAGACC TGCGGCCTGT GTGGGAATTA CAATGGCAAC CAGGGCGACG |
| 1601 | ACTTCCTTAC CCCCTCTGGG CTGGCGGAGC CCCGGGTGGA GGACTTCGGG |
| 1651 | AACGCCTGGA AGCTGCACGG GGACTGCCAG GACCTGCAGA AGCAGCACAG |
| 1701 | CGATCCCTGC GCCCTCAACC CGCGCATGAC CAGGTTCTCC GAGGAGGCGT |
| 1751 | GCGCGGTCCT GACGTCCCCC ACATTCGAGG CCTGCCATCG TGCCGTCAGC |
| 1801 | CCGCTGCCCT ACCTGCGGAA CTGCCGCTAC GACGTGTGCT CCTGCTCGGA |
| 1851 | CGGCCGCGAG TGCCTGTGCG GCGCCCTGGC CAGCTATGCC GCGGCCTGCG |
| 1901 | CGGGGAGAGG CGTGCGCGTC GCGTGGCGCG AGCCAGGCCG CTGTGAGCTG |
| 1951 | AACTGCCCGA AAGGCCAGGT GTACCTGCAG TGCGGGACCC CCTGCAACCT |
| 2001 | GACCTGCCGC TCTCTCTCTT ACCCGGATGA GGAATGCAAT GAGGCCTGCC |
| 2051 | TGGAGGGCTG CTTCTGCCCC CCAGGGCTCT ACATGGATGA GAGGGGGGAC |
| 2101 | TGCGTGCCCA AGGCCCAGTG CCCCTGTTAC TATGACGGTG AGATCTTCCA |
| 2151 | GCCAGAAGAC ATCTTCTCAG ACCATCACAC CATGTGCTAC TGTGAGGATG |
| 2201 | GCTTCATGCA CTGTACCATG AGTGGAGTCC CCGGAAGCTT GCTGCCTGAC |
| 2251 | GCTGTCCTCA GCAGTCCCCT GTCTCATCGC AGCAAAAGGA GCCTATCCTG |
| 2301 | TCGGCCCCCC ATGGTCAAGC TGGTGTGTCC CGCTGACAAC CTGCGGGCTG |
| 2351 | AAGGGCTCGA GTGTACCAAA ACGTGCCAGA ACTATGACCT GGAGTGCATG |
| 2401 | AGCATGGGCT GTGTCTCTGG CTGCCTCTGC CCCCCGGGCA TGGTCCGGCA |
| 2451 | TGAGAACAGA TGTGTGGCCC TGGAAAGGTG TCCCTGCTTC CATCAGGGCA |
| 2501 | AGGAGTATGC CCCTGGAGAA ACAGTGAAGA TTGGCTGCAA CACTTGTGTC |
| 2551 | TGTCGGGACC GGAAGTGGAA CTGCACAGAC CATGTGTGTG ATGCCACGTG |
| 2601 | CTCCACGATC GGCATGGCCC ACTACCTCAC CTTCGACGGG CTCAAATACC |
| 2651 | TGTTCCCCGG GGAGTGCCAG TACGTTCTGG TGCAGGATTA CTGCGGCAGT |
| 2701 | AACCCTGGGA CCTTTCGGAT CCTAGTGGGG AATAAGGGAT GCAGCCACCC |
| 2751 | CTCAGTGAAA TGCAAGAAAC GGGTCACCAT CCTGGTGGAG GGAGGAGAGA |
| 2801 | TTGAGCTGTT TGACGGGGAG GTGAATGTGA AGAGGCCCAT GAAGGATGAG |
| 2851 | ACTCACTTTG AGGTGGTGGA GTCTGGCCGG TACATCATTC TGCTGCTGGG |
| 2901 | CAAAGCCCTC TCCGTGGTCT GGGACCGCCA CCTGAGCATC TCCGTGGTCC |
| 2951 | TGAAGCAGAC ATACCAGGAG AAAGTGTGTG GCCTGTGTGG GAATTTTGAT |
| 3001 | GGCATCCAGA ACAATGACCT CACCAGCAGC AACCTCCAAG TGGAGGAAGA |
| 3051 | CCCTGTGGAC TTTGGGAACT CCTGGAAAGT GAGCTCGCAG TGTGCTGACA |
| 3101 | CCAGAAAAGT GCCTCTGGAC TCATCCCCTG CCACCTGCCA TAACAACATC |
| 3151 | ATGAAGCAGA CGATGGTGGA TTCCTCCTGT AGAATCCTTA CCAGTGACGT |
| 3201 | CTTCCAGGAC TGCAACAAGC TGGTGGACCC CGAGCCATAT CTGGATGTCT |
| 3251 | GCATTTACGA CACCTGCTCC TGTGAGTCCA TTGGGGACTG CGCCGCATTC |
| 3301 | TGCGACACCA TTGCTGCCTA TGCCCACGTG TGTGCCCAGC ATGGCAAGGT |
| 3351 | GGTGACCTGG AGGACGGCCA CATTGTGCCC CCAGAGCTGC GAGGAGAGGA |
| 3401 | ATCTCCGGGA GAACGGGTAT GAGGCTGAGT GGCGCTATAA CAGCTGTGCA |
| 3451 | CCTGCCTGTC AAGTCACGTG TCAGCACCCT GAGCCACTGG CCTGCCCTGT |
| 3501 | GCAGTGTGTG GAGGGCTGCC ATGCCCACTG CCCTCCAGGG AAAATCCTGG |
| 3551 | ATGAGCTTTT GCAGACCTGC GTTGACCCTG AAGACTGTCC AGTGTGTGAG |
| 3601 | GTGGCTGGCC GGCGTTTTGC CTCAGGAAAG AAAGTCACCT TGAATCCCAG |
| 3651 | TGACCCTGAG CACTGCCAGA TTTGCCACTG TGATGTTGTC AACCTCACCT |
| 3701 | GTGAAGCCTG CCAGGAGCCG GGTACATCAG AGAGCGCCAC CCCTGAAAGT |
| 3751 | GGTCCCGGGA GCGAGCCAGC CACATCTGGG TCGGAAACGC CAGGCACATC |
| 3801 | CGAGTCTGCA ACTCCCGAGT CCGGACCTGG CTCCGAGCCT GCCACTAGCG |
| 3851 | GCTCCGAGAC TCCGGGAACT TCCGAGAGCG CTACACCAGA AAGCGGACCC |
| 3901 | GGAACCAGTA CCGAACCTAG CGAGGGCTCT GCTCCGGGCA GCCCAGCCGG |
| 3951 | CTCTCCTACA TCCACGGAGG AGGGCACTTC CGAATCCGCC ACCCCGGAGT |
| 4001 | CAGGGCCAGG ATCTGAACCC GCTACCTCAG GCAGTGAGAC GCCAGGAACG |
| 4051 | AGCGAGTCCG CTACACCGGA GAGTGGGCCA GGGAGCCCTG CTGGATCTCC |
| 4101 | TACGTCCACT GAGGAAGGGT CACCAGCGGG CTCGCCCACC AGCACTGAAG |
| 4151 | AAGGTGCCTC GTCTGACAAG AACACTGGTG ATTATTACGA GGACAGTTAT |
| 4201 | GAAGATATTT CAGCATACTT GCTGAGTAAA AACAATGCCA TTGAACCAAG |
| 4251 | AAGCTTCTCT GACAAAACTC ACACATGCCC ACCGTGCCCA GCTCCAGAAC |
| 4301 | TCCTGGGCGG ACCGTCAGTC TTCCTCTTCC CCCCAAAACC CAAGGACACC |
| 4351 | CTCATGATCT CCCGGACCCC TGAGGTCACA TGCGTGGTGG TGGACGTGAG |
| 4401 | CCACGAAGAC CCTGAGGTCA AGTTCAACTG GTATGTGGAC GGCGTGGAAG |
| 4451 | TGCATAATGC CAAGACAAAG CCGCGGGAGG AGCAGTACAA CAGCACGTAC |
| 4501 | CGTGTGGTCA GCGTCCTCAC CGTCCTGCAC CAAGACTGGC TGAATGGCAA |
| 4551 | GGAGTACAAG TGCAAGGTCT CCAACAAAGC CCTCCCAGCC CCCATCGAGA |
| 4601 | AAACCATCTC CAAAGCCAAA GGGCAGCCCC GAGAACCACA GGTGTACACC |
| 4651 | CTGCCCCCAT CCCGGGATGA GCTGACCAAG AACCAAGTTA GCCTGACCTG |
| 4701 | CCTGGTCAAA GGCTTCTATC CCAGCGACAT CGCCGTGGAG TGGGAGAGCA |
| 4751 | ATGGGCAGCC GGAGAACAAC TACAAGACCA CGCCTCCCGT GTTGGACTCC |
| 4801 | GACGGCTCCT TCTTCCTCTA CTCCAAGCTC ACCGTGGACA AGAGCAGGTG |
| 4851 | GCAGCAGGGG AACGTCTTCT CATGCTCCGT GATGCATGAG GCTCTGCACA |
| 4901 | ACCACTACAC GCAGAAGAGC CTCTCCCTGT CTCCGGGTTG A |

FVIII(ELNN)-Fc: SEQ ID NO: 7

| | |
|---|---|
| TRRYYLGAVE LSWDYMQSDL GELPVDARFP PRVPKSFPEN TSVVYKKTLF VEFTDHLFNI | 60 |
| AKPRPPWMGL LGPTIQAEVY DTVVITLKNM ASHPVSLHAV GVSYWKASEG AEYDDQTSQR | 120 |
| EKEDDKVFPG GSHTYVWQVL KENGPMASDP LCLTYSYLSH VDLVKDLNSG LIGALLVCRE | 180 |
| GSLAKEKTQT LHKFILLFAV FDEGKSWHSE TKNSLMQDRD AASARAWPKM HTVNGYVNRS | 240 |
| LPGLIGCHRK SVYWHVIGMG TTPEVHSIFL EGHTFLVRNH RQASLEISPI TFLTAQTLLM | 300 |
| DLGQFLLFCH ISSHQHDGME AYVKVDSCPE EPQLRMKNNE EAEDYDDDLT DSEMDVVRFD | 360 |
| DDNSPSFIQI RSVAKKHPKT WVHYIAAEEE DWDYAPLVLA PDDRSYKSQY LNNGPQRIGR | 420 |
| KYKKVRFMAY TDETFKTREA IQHESGILGP LLYGEVGDTL LIIFKNQASR PYNIYPHGIT | 480 |
| DVRPLYSRRL PKGVKHLKDF PILPGEIFKY KWTVTVEDGP TKSDPRCLTR YYSSFVNMER | 540 |
| DLASGLIGPL LICYKESVDQ RGNQIMSDKR NVILFSVFDE NRSWYLTENI QRFLPNPAGV | 600 |
| QLEDPEFQAS NIMHSINGYV FDSLQLSVCL HEVAYWYILS IGAQTDFLSV FFSGYTFKHK | 660 |
| MVYEDTLTLF PFSGETVFMS MENPGLWILG CHNSDFRNRG MTALLKVSSC DKNTGDYYED | 720 |
| SYEDISAYLL SKNNAIEPRS FSQNGTSESA TPESGPGSEP ATSGSETPGT SESATPESGP | 780 |
| GSEPATSGSE TPGTSESATP ESGPGTSTEP SEGSAPGSPA GSPTSTEEGT SESATPESGP | 840 |
| GSEPATSGSE TPGTSESATP ESGPGSPAGS PTSTEEGSPA GSPTSTEEGT STEPSEGSAP | 900 |
| GTSESATPES GPGTSESATP ESGPGTSESA TPESGPGSEP ATSGSETPGS EPATSGSETP | 960 |
| GSPAGSPTST EEGTSTEPSE GSAPGTSTEP SEGSAPGSEP ATSGSETPGT SESATPESGP | 1020 |
| GTSTEPSEGS APASSEITRT TLQSDQEEID YDDTISVEMK KEDFDIYDED ENQSPRSFQK | 1080 |
| KTRHYFIAAV ERLWDYGMSS SPHVLRNRAQ SGSVPQFKKV VFQEFTDGSF TQPLYRGELN | 1140 |
| EHLGLLGPYI RAEVEDNIMV TFRNQASRPY SFYSSLISYE EDQRQGAEPR KNFVKPNETK | 1200 |
| TYFWKVQHHM APTKDEFDCK AWAYFSDVDL EKDVHSGLIG PLLVCHTNTL NPAHGRQVTV | 1260 |
| QEFALFFTIF DETKSWYFTE NMERNCRAPC NIQMEDPTFK ENYRFHAING YIMDTLPGLV | 1320 |
| MAQDQRIRWY LLSMGSNENI HSIHFSGHVF TVRKKEEYKM ALYNLYPGVF ETVEMLPSKA | 1380 |
| GIWRVECLIG EHLHAGMSTL FLVYSNKCQT PLGMASGHIR DFQITASGQY GQWAPKLARL | 1440 |
| HYSGSINAWS TKEPFSWIKV DLLAPMIIHG IKTQGARQKF SSLYISQFII MYSLDGKKWQ | 1500 |
| TYRGNSTGTL MVFFGNVDSS GIKHNIFNPP IIARYIRLHP THYSIRSTLR MELMGCDLNS | 1560 |
| CSMPLGMESK AISDAQITAS SYFTNMFATW SPSKARLHLQ GRSNAWRPQV NNPKEWLQVD | 1620 |
| FQKTMKVTGV TTQGVKSLLT SMYVKEFLIS SSQDGHQWTL FFQNGKVKVF QGNQDSFTPV | 1680 |
| VNSLDPPLLT RYLRIHPQSW VHQIALRMEV LGCEAQDLYD KTHTCPPCPA PELLGGPSVF | 1740 |
| LFPPKPKDTL MISRTPEVTC VVVDVSHEDP EVKFNWYVDG VEVHNAKTKP REEQYNSTYR | 1800 |
| VVSVLTVLHQ DWLNGKEYKC KVSNKALPAP IEKTISKAKG QPREPQVYTL PPSRDELTKN | 1860 |
| QVSLTCLVKG FYPSDIAVEW ESNGQPENNY KTTPPVLDSD GSFFLYSKLT VDKSRWQQGN | 1920 |
| VFSCSVMHEA LHNHYTQKSL SLSPG | 1945 |

TABLE 4
Additional chimeric protein sequences

| Description/ | | |
|---|---|---|
| SEQ ID NO. | Sequence | |
| Full length FVIII | ATRRYYLGAV ELSWDYMQSD LGELPVDARF PPRVPKSFPF NTSVVYKKTL FVEFTDHLFN | 60 |
| SEQ ID | IAKPRPPWMG LLGPTIQAEV YDTVVITLKN MASHPVSLHA VGVSYWKASE GAEYDDQTSQ | 120 |
| NO: 8 | REKEDDKVFP GGSHTYVWQV LKENGPMASD PLCLTYSYLS HVDLVKDLNS GLIGALLVCR | 180 |
| | EGSLAKEKTQ TLHKFILLFA VFDEGKSWHS ETKNSLMQDR DAASARAWPK MHTVNGYVNR | 240 |
| | SLPGLIGCHR KSVYWHVIGM GTTPEVHSIF LEGHTFLVRN HRQASLEISP ITFLTAQTLL | 300 |
| | MDLGQFLLFC HISSHQHDGM EAYVKVDSCP EEPQLRMKNN EEAEDYDDDL TDSEMDVVRF | 360 |
| | DDDNSPSFIQ IRSVAKKHPK TWVHYIAAEE EDWDYAPLVL APDDRSYKSQ YLNNGPQRIG | 420 |
| | RKYKKVRFMA YTDETFKTRE AIQHESGILG PLLYGEVGDT LLIIFKNQAS RPYNIYPHGI | 480 |
| | TDVRPLYSRR LPKGVKHLKD FPILPGEIFK YKWTVTVEDG PTKSDPRCLT RYYSSFVNME | 540 |
| | RDLASGLIGP LLICYKESVD QRGNQIMSDK RNVILFSVFD ENRSWYLTEN IQRFLPNPAG | 600 |
| | VQLEDPEFQA SNIMHSINGY VFDSLQLSVC LHEVAYWYIL SIGAQTDFLS VFFSGYTFKH | 660 |
| | KMVYEDTLTL FPFSGETVFM SMENPGLWIL GCHNSDFRNR GMTALLKVSS CDKNTGDYYE | 720 |
| | DSYEDISAYL LSKNNAIEPR SFSQNSRHPS TKQKQFNATT IPENDIEKTD PWFAHRTPMP | 780 |
| | KIQNVSSSDL LMLLRQSPTP HGLSLSDLQE AKYETFSDDP SPGAIDSNNS LSEMTHFRPQ | 840 |
| | LHHSGDMVFT PESGLQLRLN EKLGTTAATE LKKLDFKVSS TSNNLISTIP SDNLAAGTDN | 900 |
| | TSSLGPPSMP VHYDSQLDTT LFGKKSSPLT ESGGPLSLSE ENNDSKLLES GLMNSQESSW | 960 |
| | GKNVSSTESG RLFKGKRAHG PALLTKDNAL FKVSISLLKT NKTSNNSATN RKTHIDGPSL | 1020 |
| | LIENSPSVWQ NILESDTEFK KVTPLIHDRM LMDKNATALR LNHMSNKTTS SKNMEMVQQK | 1080 |
| | KEGPIPPDAQ NPDMSFFKML FLPESARWIQ RTHGKNSLNS GQGPSPKQLV SLGPEKSVEG | 1140 |
| | QNFLSEKNKV VVGKGEFTKD VGLKEMVFPS SRNLFLINLD NLHENNTHNQ EKKIQEEIEK | 1200 |
| | KETLIQENVV LPQIHTVTGT KNFMKNLFLL STRQNVEGSY DGAYAPVLQD FRSLNDSTNR | 1260 |
| | TKKHTAHFSK KGEEENLEGL GNQTKQIVEK YACTTRISPN TSQQNFVTQR SKRALKQFRL | 1320 |
| | PLEETELEKR IIVDDTSTQW SKNMKHLTPS TLTQIDYNEK EKGAITQSPL SDCLTRSHSI | 1380 |
| | PQANRSPLPI AKVSSFPSIR PIYLTRVLFQ DNSSHLPAAS YRKKDSGVQE SSHFLQGAKK | 1440 |
| | NNLSLAILTL EMTGDQREVG SLGTSATNSV TYKKVENTVL PKPDLPKTSG KVELLPKVHI | 1500 |
| | YQKDLFPTET SNGSPGHLDL VEGSLLQGTE GAIKWNEANR PGKVPFLRVA TESSAKTPSK | 1560 |
| | LLDPLAWDNH YGTQIPKEEW KSQEKSPEKT AFKKKDTILS LNACESNHAI AAINEGQNKP | 1620 |
| | EIEVTWAKQG RTERLCSQNP PVLKRHQREI TRTTLQSDQE EIDYDDTISV EMKKEDFDIY | 1680 |
| | DEDENQSPRS FQKKTRHYFI AAVERLWDYG MSSSPHVLRN RAQSGSVPQF KKVVFQEFTD | 1740 |
| | GSFTQPLYRG ELNEHLGLLG PYIRAEVEDN IMVTFRNQAS RPYSFYSSLI SYEEDQRQGA | 1800 |
| | EPRKNFVKPN ETKTYFWKVQ HHMAPTKDEF DCKAWAYFSD VDLEKDVHSG LIGPLLVCHT | 1860 |
| | NTLNPAHGRQ VTVQEFALFF TIFDETKSWY FTENMERNCR APCNIQMEDP TFKENYRFHA | 1920 |
| | INGYIMDTLP GLVMAQDQRI RWYLLSMGSN ENIHSIHFSG HVFTVRKKEE YKMALYNLYP | 1980 |
| | GVFETVEMLP SKAGIWRVEC LIGEHLHAGM STLFLVYSNK CQTPLGMASG HIRDFQITAS | 2040 |
| | GQYGQWAPKL ARLHYSGSIN AWSTKEPFSW IKVDLLAPMI IHGIKTQGAR QKFSSLYISQ | 2100 |
| | FIIMYSLDGK KWQTYRGNST GTLMVFFGNV DSSGIKHNIF NPPIIARYIR LHPTHYSIRS | 2160 |
| | TLRMELMGCD LNSCSMPLGM ESKAISDAQI TASSYFTNMF ATWSPSKARL HLQGRSNAWR | 2220 |
| | PQVNNPKEWL QVDFQKTMKV TGVTTQGVKS LLTSMYVKEF LISSSQDGHQ WTLFFQNGKV | 2280 |
| | KVFQGNQDSF TPVVNSLDPP LLTRYLRIHP QSWVHQIALR MEVLGCEAQD LY | 2332 |
| AE288 | GTSESATPES GPGSEPATSG SETPGTSESA TPESGPGSEP ATSGSETPGT SESATPESGP | 60 |
| SEQ ID NO: 9 | GTSTEPSEGS APGSPAGSPT STEEGTSESA TPESGPGSEP ATSGSETPGT SESATPESGP | 120 |
| | GSPAGSPTST EEGSPAGSPT STEEGTSTEP SEGSAPGTSE SATPESGPGT SESATPESGP | 180 |
| | GTSESATPES GPGSEPATSG SETPGSEPAT SGSETPGSPA GSPTSTEEGT STEPSEGSAP | 240 |
| | GTSTEPSEGS APGSEPATSG SETPGTSESA TPESGPGTST EPSEGSAP | 288 |
| pSYN VWF059 | TSTEEGASIS DKNTGDYYED SYEDISAYLL SKNNAIEPRS FSDKTH | |
| SEQ ID NO: 10 | | |
| pSYN VWF059A | TSTEEGASSD KNTGDYYEDS YEDISAYLLS KNNAIEPRSF SDKTH | |
| SEQ ID NO: 11 | | |
| pSYN FVIII 312 | ATRRYYLGAV ELSWDYMQSD LGELPVDARF PPRVPKSFPF NTSVVYKKTL FVEFTDHLEN | 60 |
| SEQ ID NO: | IAKPRPPWMG LLGPTIQAEV YDTVVITLKN MASHPVSLHA VGVSYWKASE GAEYDDQTSQ | 120 |
| 12 | REKEDDKVFP GGSHTYVWQV LKENGPMASD PLCLTYSYLS HVDLVKDLNS GLIGALLVCR | 180 |
| | EGSLAKEKTQ TLHKFILLFA VFDEGKSWHS ETKNSLMQDR DAASARAWPK MHTVNGYVNR | 240 |
| | SLPGLIGCHR KSVYWHVIGM GTTPEVHSIF LEGHTFLVRN HRQASLEISP ITFLTAQTLL | 300 |
| | MDLGQFLLFC HISSHQHDGM EAYVKVDSCP EEPQLRMKNN EEAEDYDDDL TDSEMDVVRF | 360 |
| | DDDNSPSFIQ IRSVAKKHPK TWVHYIAAEE EDWDYAPLVL APDDRSYKSQ YLNNGPQRIG | 420 |
| | RKYKKVRFMA YTDETFKTRE AIQHESGILG PLLYGEVGDT LLIIFKNQAS RPYNIYPHGI | 480 |
| | TDVRPLYSRR LPKGVKHLKD FPILPGEIFK YKWTVTVEDG PTKSDPRCLT RYYSSFVNME | 540 |
| | RDLASGLIGP LLICYKESVD QRGNQIMSDK RNVILFSVED ENRSWYLTEN IQRFLPNPAG | 600 |
| | VQLEDPEFQA SNIMHSINGY VEDSLQLSVC LHEVAYWYIL SIGAQTDFLS VFFSGYTFKH | 660 |
| | KMVYEDTLTL FPFSGETVFM SMENPGLWIL GCHNSDERNR GMTALLKVSS CDKNTGDYYE | 720 |
| | DSYEDISAYL LSKNNAIEPR SFSQNGTSES ATPESGPGSE PATSGSETPG TSESATPESG | 780 |
| | PGSEPATSGS ETPGTSESAT PESGPGTSTE PSEGSAPGSP AGSPTSTEEG TSESATPESG | 840 |
| | PGSEPATSGS ETPGTSESAT PESGPGSPAG SPTSTEEGSP AGSPTSTEEG TSTEPSEGSA | 900 |
| | PGTSESATPE SGPGTSESAT PESGPGTSES ATPESGPGSE PATSGSETPG SEPATSGSET | 960 |
| | PGSPAGSPTS TEEGTSTEPS EGSAPGTSTE PSEGSAPGSE PATSGSETPG TSESATPESG | 1020 |
| | PGTSTEPSEG SAPASSEITR TTLQSDQEEI DYDDTISVEM KKEDFDIYDE DENQSPRSFQ | 1080 |
| | KKTRHYFIAA VERLWDYGMS SSPHVLRNRA QSGSVPQFKK VVFQEFTDGS FTQPLYRGEL | 1140 |
| | NEHLGLLGPY IRAEVEDNIM VTERNQASRP YSFYSSLISY EEDQRQGAEP RKNFVKPNET | 1200 |
| | KTYFWKVQHH MAPTKDEFDC KAWAYFSDVD LEKDVHSGLI GPLLVCHTNT LNPAHGRQVT | 1260 |
| | VQEFALFFTI FDETKSWYFT ENMERNCRAP CNIQMEDPTF KENYRFHAIN GYIMDTLPGL | 1320 |
| | VMAQDQRIRW YLLSMGSNEN IHSIHFSGHV FTVRKKEEYK MALYNLYPGV FETVEMLPSK | 1380 |
| | AGIWRVECLI GEHLHAGMST LFLVYSNKCQ TPLGMASGHI RDFQITASGQ YGQWAPKLAR | 1440 |
| | LHYSGSINAW STKEPFSWIK VDLLAPMIIH GIKTQGARQK FSSLYISQFI IMYSLDGKKW | 1500 |
| | QTYRGNSTGT LMVFFGNVDS SGIKHNIFNP PIIARYIRLH PTHYSIRSTL RMELMGCDLN | 1560 |
| | SCSMPLGMES KAISDAQITA SSYFTNMFAT WSPSKARLHL QGRSNAWRPQ VNNPKEWLQV | 1620 |
| | DFQKTMKVTG VTTQGVKSLL TSMYVKEFLI SSSQDGHQWT LFFQNGKVKV FQGNQDSFTP | 1680 |
| | VVNSLDPPLL TRYLRIHPQS WVHQIALRME VLGCEAQDLY DKTHTCPPCP APELLGGPSV | 1740 |
| | FLFPPKPKDT LMISRTPEVT CVVVDVSHED PEVKFNWYVD GVEVHNAKTK PREEQYNSTY | 1800 |
| | RVVSVLTVLH QDWINGKEYK CKVSNKALPA PIEKTISKAK GQPREPQVYT LPPSRDELTK | 1860 |
| | NQVSLTCLVK GFYPSDIAVE WESNGQPENN YKTTPPVLDS DGSFFLYSKL TVDKSRWQQG | 1920 |
| | NVFSCSVMHE ALHNHYTQKS LSLSPGK | |
| ELNN_AE288_2 | GSPAGSPTST EEGTSESATP ESGPGSEPAT SGSETPGTSE SATPESGPGT STEPSEGSAP | 60 |
| SEQ ID NO: 13 | GTSTEPSEGS APGTSTEPSE GSAPGTSTEP SEGSAPGTST EPSEGSAPGT STEPSEGSAP | 120 |
| | GSPAGSPTST EEGTSTEPSE GSAPGTSESA TPESGPGSEP ATSGSETPGT SESATPESGP | 180 |
| | GSEPATSGSE TPGTSESATP ESGPGTSTEP SEGSAPGTSE SATPESGPGS PAGSPTSTEE | 240 |
| | GSPAGSPTST EEGSPAGSPT STEEGTSESA TPESGPGTST EPSEGSAP | 288 |
| ELNN AE144_5A | TSESATPESG PGSEPATSGS ETPGTSESAT PESGPGSEPA TSGSETPGTS ESATPESGPG | 60 |
| SEQ ID NO: 14 | TSTEPSEGSA PGSPAGSPTS TEEGTSESAT PESGPGSEPA TSGSETPGTS ESATPESGPG | 120 |
| | SPAGSPTSTE EGSPAGSPTS TEEG | |
| a2 Linker of | DKNTGDYYED SYEDISAYLL SKNNAIEPRS FS | 32 |
| chimeric protein | | |
| SEQ ID NO: 15 | | |
| Signal Peptide of | MQIELSTCFFLCLLRFCFS | |
| FVIII | | |
| SEQ ID NO: 16 | | |
| FVIII fragment 1 | ATRRYYLGAV ELSWDYMQSD LGELPVDARF PPRVPKSFPF NTSVVYKKTL FVEFTDHLEN | 60 |
| of chimeric | IAKPRPPWMG LLGPTIQAEV YDTVVITLKN MASHPVSLHA VGVSYWKASE GAEYDDQTSQ | 120 |
| protein | REKEDDKVFP GGSHTYVWQV LKENGPMASD PLCLTYSYLS HVDLVKDLNS GLIGALLVCR | 180 |
| SEQ ID NO. 17 | EGSLAKEKTQ TLHKFILLFA VFDEGKSWHS ETKNSLMQDR DAASARAWPK MHTVNGYVNR | 240 |
| | SLPGLIGCHR KSVYWHVIGM GTTPEVHSIF LEGHTFLVRN HRQASLEISP ITFLTAQTLL | 300 |
| | MDLGQFLLFC HISSHQHDGM EAYVKVDSCP EEPQLRMKNN EEAEDYDDDL TDSEMDVVRF | 360 |
| | DDDNSPSFIQ IRSVAKKHPK TWVHYIAAEE EDWDYAPLVL APDDRSYKSQ YLNNGPQRIG | 420 |
| | RKYKKVRFMA YTDETFKTRE AIQHESGILG PLLYGEVGDT LLIIFKNQAS RPYNIYPHGI | 480 |
| | TDVRPLYSRR LPKGVKHLKD FPILPGEIFK YKWTVTVEDG PTKSDPRCLT RYYSSFVNME | 540 |
| | RDLASGLIGP LLICYKESVD QRGNQIMSDK RNVILFSVED ENRSWYLTEN IQRFLPNPAG | 600 |
| | VQLEDPEFQA SNIMHSINGY VFDSLQLSVC LHEVAYWYIL SIGAQTDFLS VFFSGYTFKH | 660 |
| | KMVYEDTLTL FPFSGETVFM SMENPGLWIL GCHNSDFRNR GMTALLKVSS CDKNTGDYYE | 720 |
| | DSYEDISAYL LSKNNAIEPR SFSQN | |
| FVIII fragment 2 | EITRTTLQSD QEEIDYDDTI SVEMKKEDED IYDEDENQSP RSFQKKTRHY FIAAVERLWD | 60 |
| of chimeric | YGMSSSPHVL RNRAQSGSVP QFKKVVFQEF TDGSFTQPLY RGELNEHLGL LGPYIRAEVE | 120 |
| protein | DNIMVTERNQ ASRPYSFYSS LISYEEDQRQ GAEPRKNFVK PNETKTYFWK VQHHMAPTKD | 180 |
| SEQ ID NO. 18 | EFDCKAWAYF SDVDLEKDVH SGLIGPLLVC HTNTLNPAHG RQVTVQEFAL FFTIFDETKS | 240 |
| | WYFTENMERN CRAPCNIQME DPTFKENYRF HAINGYIMDT LPGLVMAQDQ RIRWYLLSMG | 300 |
| | SNENIHSIHF SGHVFTVRKK EEYKMALYNL YPGVFETVEM LPSKAGIWRV ECLIGEHLHA | 360 |
| | GMSTLFLVYS NKCQTPLGMA SGHIRDFQIT ASGQYGQWAP KLARLHYSGS INAWSTKEPF | 420 |
| | SWIKVDLLAP MIIHGIKTQG ARQKFSSLYI SQFIIMYSLD GKKWQTYRGN STGTLMVFFG | 480 |
| | NVDSSGIKHN IFNPPIIARY IRLHPTHYSI RSTLRMELMG CDLNSCSMPL GMESKAISDA | 540 |
| | QITASSYFTN MFATWSPSKA RLHLQGRSNA WRPQVNNPKE WLQVDFQKTM KVTGVTTQGV | 600 |
| | KSLLTSMYVK EFLISSSQDG HQWTLFFQNG KVKVFQGNQD SFTPVVNSLD PPLLTRYLRI | 660 |
| | HPQSWVHQIA LRMEVLGCEA QDLY | |
| VWF Signal | MIPARFAGVL LALALILPGT LC | |
| Peptide | | |
| SEQ ID NO: 19 | | |
| VWF D1D2 domain | AEGTRGRSST ARCSLFGSDF VNTFDGSMYS FAGYCSYLLA GGCQKRSFSI IGDFQNGKRV | 60 |
| of chimeric | SLSVYLGEFF DIHLFVNGTV TQGDQRVSMP YASKGLYLET EAGYYKLSGE AYGFVARIDG | 120 |
| protein | SGNFQVLLSD RYFNKTCGLC GNFNIFAEDD FMTQEGTLTS DPYDFANSWA LSSGEQWCER | 180 |
| SEQ ID NO: 20 | ASPPSSSCNI SSGEMQKGLW EQCQLLKSTS VFARCHPLVD PEPFVALCEK TLCECAGGLE | 240 |
| | CACPALLEYA RTCAQEGMVL YGWTDHSACS PVCPAGMEYR QCVSPCARTC QSLHINEMCQ | 300 |
| | ERCVDGCSCP EGQLLDEGLC VESTECPCVH SGKRYPPGTS LSRDCNTCIC RNSQWICSNE | 360 |
| | ECPGECLVTG QSHFKSFDNR YFTFSGICQY LLARDCQDHS FSIVIETVQC ADDRDAVCTR | 420 |
| | SVTVRLPGLH NSLVKLKHGA GVAMDGQDIQ LPLLKGDLRI QHTVTASVRL SYGEDLQMDW | 480 |
| | DGRGRLLVKL SPVYAGKTCG LCGNYNGNQG DDFLTPSGLA EPRVEDFGNA WKLHGDCQDL | 540 |
| | QKQHSDPCAL NPRMTRFSEE ACAVLTSPTF EACHRAVSPL PYLRNCRYDV CSCSDGRECL | 600 |
| | CGALASYAAA CAGRGVRVAW REPGRCELNC PKGQVYLQCG TPCNLTCRSL SYPDEECNEA | 660 |
| | CLEGCFCPPG LYMDERGDCV PKAQCPCYYD GEIFQPEDIF SDHHTMCYCE DGFMHCTMSG | 720 |
| | VPGSLLPDAV LSSPLSHRSK R | |
| VWF D' domain of | SLSCRPPMVK LVCPADNLRA EGLECTKTCQ NYDLECMSMG CVSGCLCPPG MVRHENRCVA | 60 |
| chimeric protein | LERCPCFHQG KEYAPGETVK IGCNTCVCRD RKWNCTDHVC DAT | 103 |
| SEQ ID NO: 21 | | |
| VWF D3 domain of | CSTIGMAHYL TFDGLKYLFP GECQYVLVQD YCGSNPGTFR ILVGNKGCSH PSVKCKKRVT | 60 |
| chimeric protein | ILVEGGEIEL FDGEVNVKRP MKDETHFEVV ESGRYIILLL GKALSVVWDR HLSISVVLKQ | 120 |
| SEQ ID NO: 22 | TYQEKVCGLC GNFDGIQNND LTSSNLQVEE DPVDFGNSWK VSSQCADTRK VPLDSSPATC | 180 |
| | HNNIMKQTMV DSSCRILISD VFQDCNKLVD PEPYLDVCIY DTCSCESIGD CAAFCDTIAA | 240 |
| | YAHVCAQHGK VVTWRTATLC PQSCEERNLR ENGYEAEWRY NSCAPACQVT CQHPEPLACP | 300 |
| | VQCVEGCHAH CPPGKILDEL LQTCVDPEDC PVCEVAGRRF ASGKKVTLNP SDPEHCQICH | 360 |
| | CDVVNLTCEA CQEP | 374 |
| Fc region | DKTHTCPPCP APELLGGPSV FLFPPKPKDT LMISRTPEVT CVVVDVSHED PEVKFNWYVD | 60 |
| SEQ ID NO: 23 | GVEVHNAKTK PREEQYNSTY RVVSVLTVLH QDWLNGKEYK CKVSNKALPA PIEKTISKAK | 120 |
| | GQPREPQVYT LPPSRDELTK NQVSLTCLVK GFYPSDIAVE WESNGQPENN YKTTPPVLDS | 180 |
| | DGSFFLYSKL TVDKSRWQQG NVFSCSVMHE ALHNHYTQKS LSLSPG | |
| ELNN AE288_3 | GTSESATPES GPGSEPATSG SETPGTSESA TPESGPGSEP ATSGSETPGT SESATPESGP | 60 |
| SEQ ID NO: 24 | GTSTEPSEGS APGSPAGSPT STEEGTSESA TPESGPGSEP ATSGSETPGT SESATPESGP | 120 |
| | GSPAGSPTST EEGSPAGSPT STEEGTSTEP SEGSAPGTSE SATPESGPGT SESATPESGP | 180 |
| | GTSESATPES GPGSEPATSG SETPGSEPAT SGSETPGSPA GSPTSTEEGT STEPSEGSAP | 240 |
| | GTSTEPSEGS APGSEPATSG SETPGTSESA TPESGPGTST EPSEGSAPAS S | 291 |

1. A pharmaceutical composition comprising:
(a) a chimeric protein comprising a first polypeptide chain which comprises a Factor VIII (“FVIII”) protein and a first immunoglobulin (“Ig”) constant region or a portion thereof, and
a second polypeptide chain which comprises a von Willebrand Factor (“VWF”) protein and a second Ig constant region or a portion thereof;
(b) sucrose;
(c) histidine;
(d) arginine;
(e) calcium chloride; and
(f) a poloxamer.

2. The pharmaceutical composition of claim 1, wherein the pharmaceutical composition comprises about 1% (w/v) to about 7.5% (w/v) sucrose.

3. The pharmaceutical composition of claim 1, wherein the pharmaceutical composition comprises about 1% (w/v) to about 5% (w/v) sucrose.

4. The pharmaceutical composition of claim 1, wherein the poloxamer is poloxamer 188 (P188).

5. The pharmaceutical composition of claim 1, wherein the pharmaceutical composition comprises about 5 mM to about 15 mM histidine.

6. The pharmaceutical composition of claim 1, wherein the pharmaceutical composition comprises about 200 mM to about 300 mM arginine.

7. The pharmaceutical composition of claim 1, wherein the pharmaceutical composition comprises about 2.5 mM to about 10 mM calcium chloride.

8. The pharmaceutical composition of claim 1, wherein the pharmaceutical composition comprises about 0.01% (w/v) to about 1.0% (w/v) poloxamer 188 (P188).

9. The pharmaceutical composition of claim 1, wherein the pharmaceutical composition comprises:
(a) about 1% (w/v) to about 5% (w/v) sucrose;
(b) about 5 mM to about 15 mM histidine;
(c) about 200 mM to about 300 mM arginine;
(d) about 2.5 mM to about 10 mM calcium chloride; and
(e) about 0.01% (w/v) to about 1.0% (w/v) poloxamer 188.

10. The pharmaceutical composition of claim 9, wherein the pharmaceutical composition comprises:
(a) about 5% (w/v) sucrose;
(b) about 10 mM histidine;
(c) about 250 mM arginine;
(d) about 5 mM calcium chloride; and
(e) about 0.1% (w/v) poloxamer 188.

11-31. (canceled)

32. The pharmaceutical composition of claim 1, wherein the pharmaceutical composition has a pH of about 6.5 to about 7.5.

33-44. (canceled)

45. The pharmaceutical composition of claim 1, wherein the first polypeptide chain comprises the amino acid sequence set forth as SEQ ID NO: 1 and the second polypeptide chain comprises the amino acid sequence set forth as SEQ ID NO: 2, wherein the first polypeptide chain and the second polypeptide chain are covalently linked by two disulfide bonds between Fc domains in the first and second polypeptide chains.

46-47. (canceled)

48. The pharmaceutical composition of claim 1, wherein the pharmaceutical composition comprises 75 IU/mL to 2,000 IU/mL of the chimeric protein.

49-51. (canceled)

52. A method of treating hemophilia A in a subject in need thereof, comprising administering to the subject an effective amount of the pharmaceutical composition of claim 1.

53-54. (canceled)

55. The method of claim 52, wherein the pharmaceutical composition is administered intravenously at a dose of 20 IU/kg to 70 IU/kg.

56. (canceled)

57. The method of claim 52, wherein the pharmaceutical composition is administered intravenously once every 7-10 days.

58. (canceled)

59. A pharmaceutical kit comprising:
(i) a first container comprising a lyophilized pharmaceutical composition comprising
(a) a chimeric protein comprising
a first polypeptide chain which comprises a Factor VIII (“FVIII”) protein or a portion thereof and a first immunoglobulin (“Ig”) constant region or a portion thereof, and
a second polypeptide chain which comprises a von Willebrand Factor (“VWF”) protein and a second Ig constant region or a portion thereof;
(b) sucrose;
(c) histidine;
(d) arginine;
(e) calcium chloride; and
(f) poloxamer 188, and
(ii) a second container comprising sterile water.

60. The pharmaceutical kit of claim 59, wherein the lyophilized pharmaceutical composition comprises:
(a) about 10 mg to about 200 mg sucrose;
(b) about 2.5 mg to about 7.5 mg histidine;
(c) about 140 mg to about 200 mg arginine;
(d) about 1.5 mg to about 5 mg calcium chloride; and
(e) about 1 mg to about 10 mg poloxamer 188.

61-68. (canceled)

69. The pharmaceutical kit of claim 59, wherein the second container comprises about 2 mL to about 5 mL of the sterile water.

70-79. (canceled)

80. A method of treating hemophilia A in a subject in need thereof, comprising combining the lyophilized pharmaceutical composition and the sterile water of the kit of claim 59, and administering to the subject an effective amount of the resulting combination.

81-83. (canceled)
